(19) United States
(12) Patent Application Publication
(10) Pub. No.: US 2022/0296616 A1
(43) Pub. Date: Sep. 22, 2022

(54) SHP1 AND SHP2 INHIBITORS AND THEIR METHODS OF USE

(71) Applicant: Tiaki Therapeutics Inc., Cambridge, MA (US)

(72) Inventors: Thomas Allen Miller, Cambridge, MA (US); Jonathan Max Levenson, Cambridge, MA (US); Lauren Martens, Cambridge, MA (US); Kelley Cronin Larson, Cambridge, MA (US); Suzanne Bruhn, Cambridge, MA (US)

(73) Assignee: Tiaki Therapeutics Inc., Cambridge, MA (US)

(21) Appl. No.: 17/837,458

(22) Filed: Jun. 10, 2022

Related U.S. Application Data
(63) Continuation of application No. PCT/US2020/064665, filed on Dec. 11, 2020.
(60) Provisional application No. 62/946,850, filed on Dec. 11, 2019.

(51) Int. Cl. A61K 31/655; A61K 31/497; A61K 31/4985; A61P 25/28

(52) U.S. Cl. CPC A61K 31/655; A61K 31/497; A61K 31/4985; A61P 25/28

(57) ABSTRACT

Described herein, in one embodiment, are methods of treating neuroinflammation and other disorders in a subject in need thereof, comprising administering to the subject an effective amount of a compound described herein, e.g., a SHP1 inhibitor or SHP2 inhibitor, or pharmaceutical composition thereof.

### CROSS-REFERENCE TO RELATED APPLICATION

This application is a continuation of International Patent Application No. PCT/US2020/064665, filed Dec. 11, 2020, which claims priority from and the benefit of U.S. Provisional Patent Application No. 62/946,850, filed Dec. 11, 2019. The entire contents of these applications are incorporated herein by reference.

### BACKGROUND

SHP1 and SHP2 are non-receptor protein tyrosine phosphatases encoded by the PTPN6 and PTPN11 genes, respectively, and contribute to multiple cellular functions including proliferation, differentiation, cell cycle maintenance and migration. SHP2 is involved in signaling through the RAS-mitogen-activated protein kinase (MAPK), the JAK-STAT and/or the phosphoinositol 3-kinase-AKT pathways.

SHP1/SHP2 has two N-terminal Src homology 2 domains (N-SH2 and C-SH2), a catalytic domain (PTP), and a C-terminal tail. The two SHP2 domains control the subcellular localization and functional regulation of SHP2. The molecule exists in an inactive, self-inhibited conformation stabilized by a binding network involving residues from both the N-SH2 and PTP domains. Stimulation by, for example, cytokines or growth factors acting through RTKs leads to exposure of the catalytic site resulting in enzymatic activation of SHP2. Mutations in the PTPN11 gene and subsequently in SHP2 have been identified in several human developmental diseases, such as Noonan Syndrome and Leopard Syndrome. Some of these mutations destabilize the autoinhibited conformation of SHP2 and promote autoactivation or enhanced growth factor-driven activation of SHP2.

Microglia, the resident CNS immune cells, are multifunctional and display robust plasticity of functional state. One of the functions of microglia is phagocytic clearance of extracellular material such as protein aggregates and cellular debris. Overall phagocytic activity is controlled by 2 families of receptors, Immunoreceptor Tyrosine-based Activation Motif (ITAM) and Immunoreceptor Tyrosine-based Inhibition Motif (ITIM). The ITAM family generally signals through tyrosine kinases, and the ITIM family generally signals through phosphatases like PTPN6 and PTPN11. These signaling cascades converge on Spleen Tyrosine Kinase (Syk), which signals through the PI3-kinase signaling cascade to modulate overall phagocytic capacity and rates.

Both SHP1 and SHP2 accordingly represent highly attractive targets for the development of novel therapies for the treatment of various diseases, including CNS-related disorders.

### SUMMARY

Provided herein, in part, are methods of treating or preventing inflammation and other disorders in a subject in need thereof. In an embodiment, the present disclosure provides a method of treating or preventing disorders associated with PTPN6 mutations in a subject in need thereof, comprising administering to the subject an effective amount of a SHP1 inhibitor described herein, e.g., an allosteric SHP1 inhibitor described herein, or pharmaceutical composition thereof. In an embodiment, the present disclosure provides a method of treating or preventing disorders associated with PTPN11 mutations in a subject in need thereof, comprising administering to the subject an effective amount of a SHP2 inhibitor described herein, e.g., an allosteric SHP2 inhibitor described herein, or pharmaceutical composition thereof.

The present disclosure also provides methods of treating or preventing neuroinflammation and related disorders in a subject in need thereof, the method comprising administering to the subject an effective amount of a compound described herein, e.g., a SHP1 inhibitor described herein or a SHP2 inhibitor described herein, or pharmaceutical composition thereof.

Also described herein is a method of treating or preventing neuroinflammation in a subject in need thereof, the method comprising administering to the subject an effective amount of a compound described herein, e.g., a SHP1 inhibitor described herein or a SHP2 inhibitor described herein, or pharmaceutical composition thereof, with the proviso that the SHP2 inhibitor is not: (S)-4-(((S)-1-(12-azanyl)-3-(4-(difluoro(phosphono)methyl)phenyl)-1-oxopropan-2-yl)amino)-3-((S)-3-(4-(difluoro(phosphono)methyl)phenyl)-2-pentadecanamidopropanamido)-4-oxobutanoic acid, ((4-((S)-3-(((S)-1-amino-6-(4-ethylbenzamido)-1-oxohexan-2-yl)amino)-2-((S)-2-(2-(((1R,2R,5S)-2-isopropyl-5-methylcyclohexyl)oxy)acetamido)-3-phenylpropanamido)-3-oxopropyl)phenyl)difluoromethyl)phosphonic acid, ((4-((S)-3-(((S)-1-amino-3-(2-(4-hydroxy-3-methoxyphenyl)acetamido)-1-oxopropan-2-yl)amino)-5-(3-iodobenzamido)-1-oxopentan-2-yl)amino)-6-hydroxy-3-iodo-1-methyl-2-(3-(2-oxo-2-((4-(thiophen-3-yl)phenyl)amino)acetamido)phenyl)-1H-indole-5-carboxylic acid, ((4-((S)-3-(((S)-1-(((S)-1-amino-3-(2-(4-hydroxy-3-methoxyphenyl)acetamido)-1-oxopropan-2-yl)amino)-5-(3-iodobenzamido)-1-oxopentan-2-yl)amino)-2-(3-bromo-4-methylbenzamido)-3-oxopropyl)phenyl)difluoromethyl)difluoromethyl)phosphonic acid, 3-((3-Chlorophenyl)ethynyl)-2-(4-(2-(cyclopropylamino)-2-oxoethoxy)phenyl)-6-hydroxybenzofuran-5-carboxylic acid, or 2-(3-(2-(3-bromo-5-iodobenzamido)acetamido)phenyl)-6-hydroxy-3-iodo-1-methyl-1H-indole-5-caroxylic acid.

In one embodiment, described herein is a method of treating or preventing neuroinflammation in a subject in need thereof, the method comprising administering to the subject an effective amount of a SHP2 inhibitor having the structure:

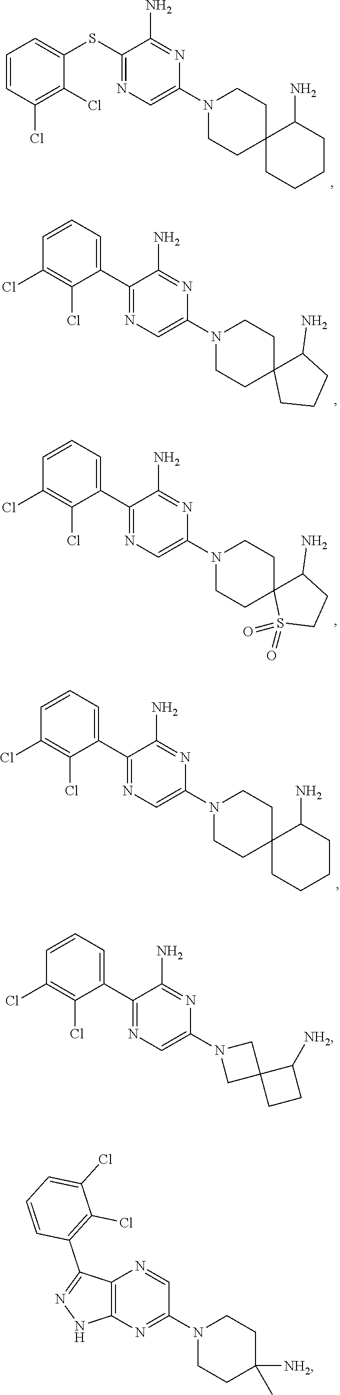 [SMILES: Nc1nc(N2CCC3(CCCCC3N)CC2)cnc1Sc1cccc(Cl)c1Cl ; Nc1nc(N2CCC3(CCCC3N)CC2)cnc1-c1cccc(Cl)c1Cl ; Nc1nc(N2CCC3(CC2)C(N)CCS3(=O)=O)cnc1-c1cccc(Cl)c1Cl ; Nc1nc(N2CCC3(CCCCC3N)CC2)cnc1-c1cccc(Cl)c1Cl ; Nc1nc(N2CC3(CCC3N)C2)cnc1-c1cccc(Cl)c1Cl ; CC1(N)CCN(c2cnc3c(-c4cccc(Cl)c4Cl)n[nH]c3n2)CC1]

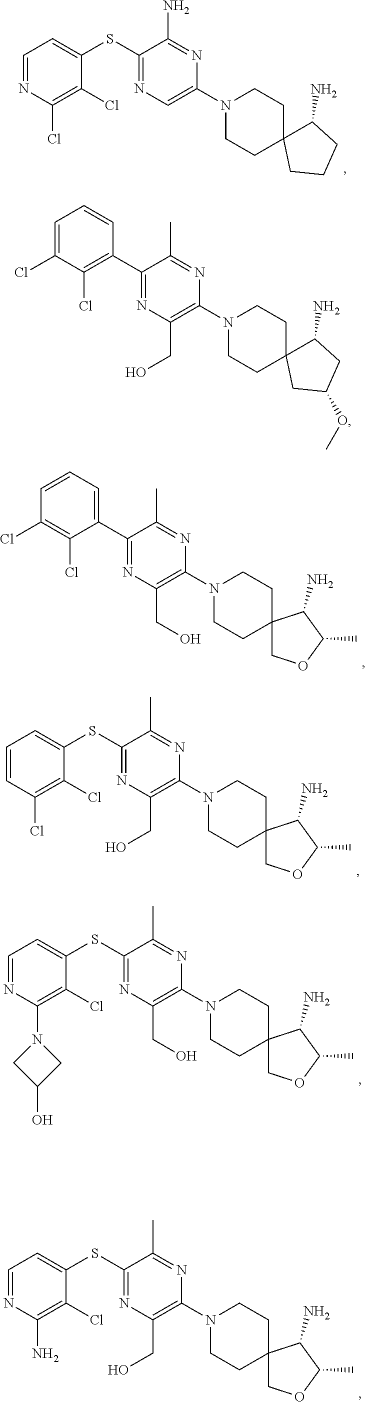 [SMILES: Nc1nc(N2CCC3(CCC[C@H]3N)CC2)cnc1Sc1ccnc(Cl)c1Cl ; CO[C@H]1C[C@@H](N)C2(CCN(c3nc(C)c(-c4cccc(Cl)c4Cl)nc3CO)CC2)C1 ; Cc1nc(N2CCC3(CC2)CO[C@@H](C)[C@H]3N)c(CO)nc1-c1cccc(Cl)c1Cl ; Cc1nc(N2CCC3(CC2)CO[C@@H](C)[C@H]3N)c(CO)nc1Sc1cccc(Cl)c1Cl ; Cc1nc(N2CCC3(CC2)CO[C@@H](C)[C@H]3N)c(CO)nc1Sc1ccnc(N2CC(O)C2)c1Cl ; Cc1nc(N2CCC3(CC2)CO[C@@H](C)[C@H]3N)c(CO)nc1Sc1ccnc(N)c1Cl]

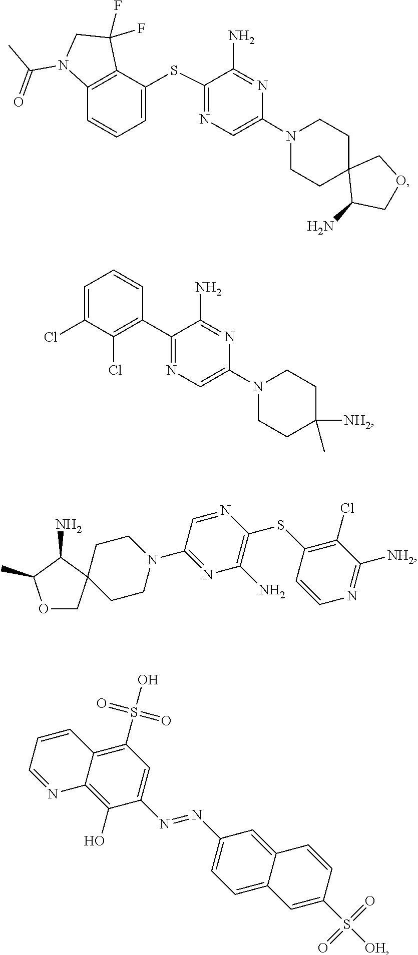 [SMILES: CC(=O)N1CC(F)(F)c2c(Sc3ncc(N4CCC5(CC4)COC[C@H]5N)nc3N)cccc21 ; CC1(N)CCN(c2cnc(-c3cccc(Cl)c3Cl)c(N)n2)CC1 ; C[C@@H]1OCC2(CCN(c3cnc(Sc4ccnc(N)c4Cl)c(N)n3)CC2)[C@@H]1N ; O=S(=O)(O)c1ccc2cc(/N=N/c3cc(S(=O)(=O)O)c4cccnc4c3O)ccc2c1]

or a pharmaceutically acceptable salt thereof.

In one embodiment, described herein is a method of treating or preventing a neuroinflammatory disorder in a subject in need thereof, the method comprising administering to the subject an effective amount of a SHP2 inhibitor having the structure:

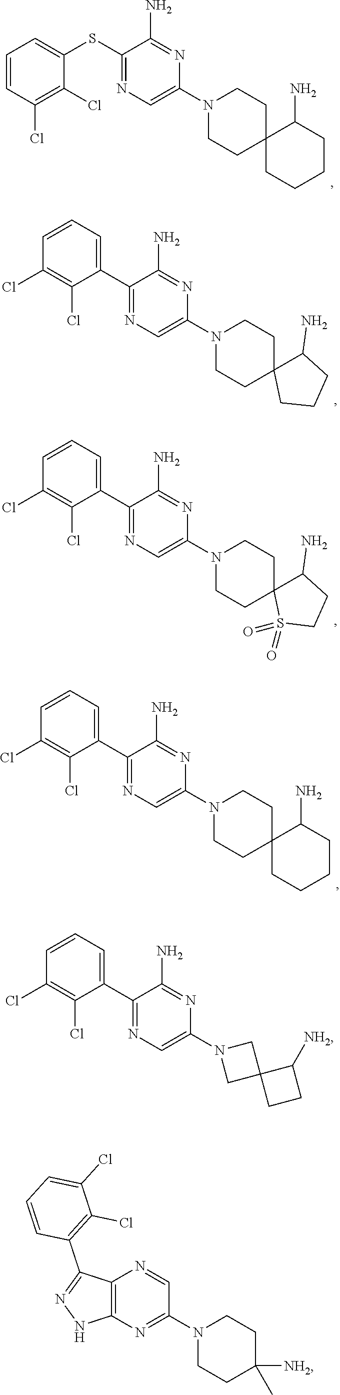 [SMILES: Nc1nc(N2CCC3(CCCCC3N)CC2)cnc1Sc1cccc(Cl)c1Cl ; Nc1nc(N2CCC3(CCCC3N)CC2)cnc1-c1cccc(Cl)c1Cl ; Nc1nc(N2CCC3(CC2)C(N)CCS3(=O)=O)cnc1-c1cccc(Cl)c1Cl ; Nc1nc(N2CCC3(CCCCC3N)CC2)cnc1-c1cccc(Cl)c1Cl ; Nc1nc(N2CC3(CCC3N)C2)cnc1-c1cccc(Cl)c1Cl ; CC1(N)CCN(c2cnc3c(-c4cccc(Cl)c4Cl)n[nH]c3n2)CC1]

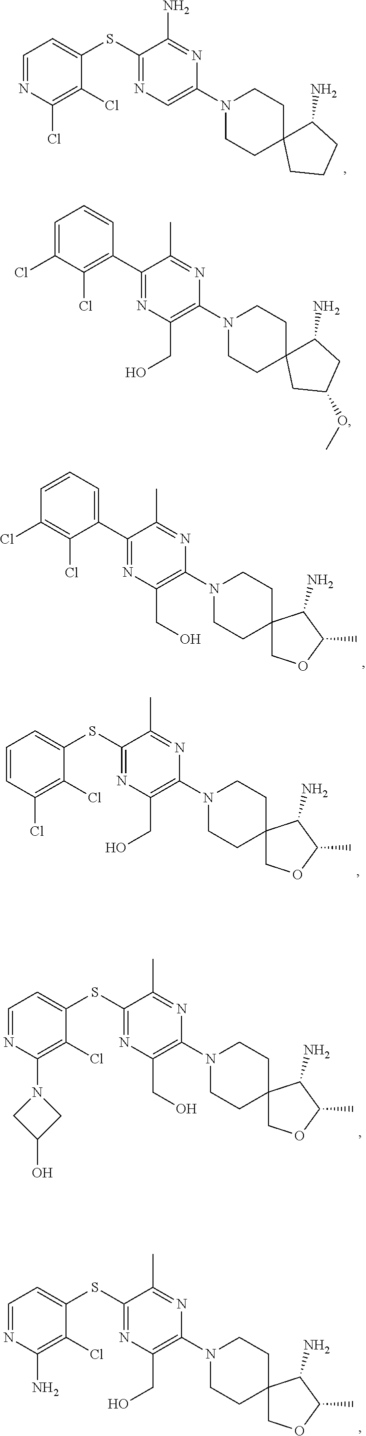 [SMILES: Nc1nc(N2CCC3(CCC[C@H]3N)CC2)cnc1Sc1ccnc(Cl)c1Cl ; CO[C@H]1C[C@@H](N)C2(CCN(c3nc(C)c(-c4cccc(Cl)c4Cl)nc3CO)CC2)C1 ; Cc1nc(N2CCC3(CC2)CO[C@@H](C)[C@H]3N)c(CO)nc1-c1cccc(Cl)c1Cl ; Cc1nc(N2CCC3(CC2)CO[C@@H](C)[C@H]3N)c(CO)nc1Sc1cccc(Cl)c1Cl ; Cc1nc(N2CCC3(CC2)CO[C@@H](C)[C@H]3N)c(CO)nc1Sc1ccnc(N2CC(O)C2)c1Cl ; Cc1nc(N2CCC3(CC2)CO[C@@H](C)[C@H]3N)c(CO)nc1Sc1ccnc(N)c1Cl]

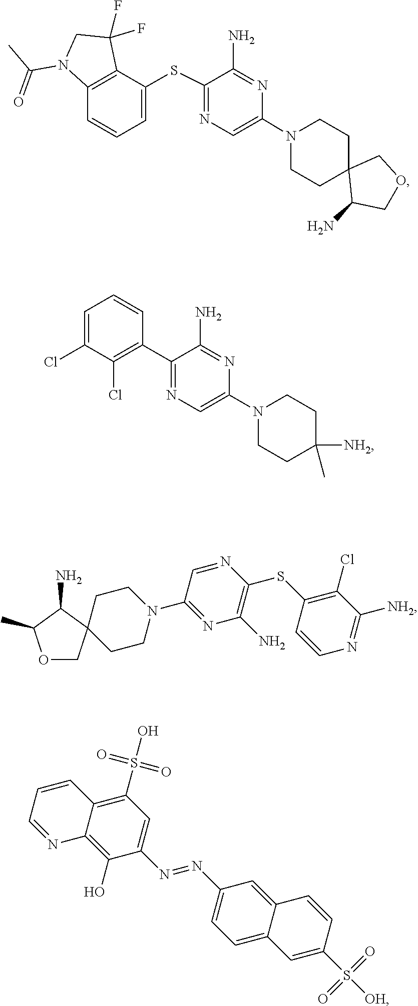 [SMILES: CC(=O)N1CC(F)(F)c2c(Sc3ncc(N4CCC5(CC4)COC[C@H]5N)nc3N)cccc21 ; CC1(N)CCN(c2cnc(-c3cccc(Cl)c3Cl)c(N)n2)CC1 ; C[C@@H]1OCC2(CCN(c3cnc(Sc4ccnc(N)c4Cl)c(N)n3)CC2)[C@@H]1N ; O=S(=O)(O)c1ccc2cc(/N=N/c3cc(S(=O)(=O)O)c4cccnc4c3O)ccc2c1]

or a pharmaceutically acceptable salt thereof.

In one embodiment, described herein is a method of treating or preventing a disorder associated with neuroinflammation in a subject in need thereof, the method comprising administering to the subject an effective amount of a SHP2 inhibitor having the structure:

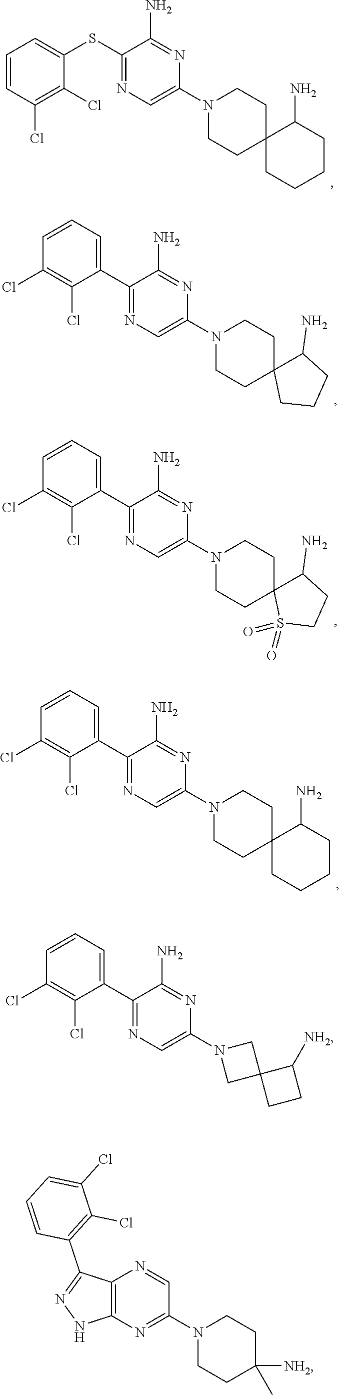 [SMILES: Nc1nc(N2CCC3(CCCCC3N)CC2)cnc1Sc1cccc(Cl)c1Cl ; Nc1nc(N2CCC3(CCCC3N)CC2)cnc1-c1cccc(Cl)c1Cl ; Nc1nc(N2CCC3(CC2)C(N)CCS3(=O)=O)cnc1-c1cccc(Cl)c1Cl ; Nc1nc(N2CCC3(CCCCC3N)CC2)cnc1-c1cccc(Cl)c1Cl ; Nc1nc(N2CC3(CCC3N)C2)cnc1-c1cccc(Cl)c1Cl ; CC1(N)CCN(c2cnc3c(-c4cccc(Cl)c4Cl)n[nH]c3n2)CC1]

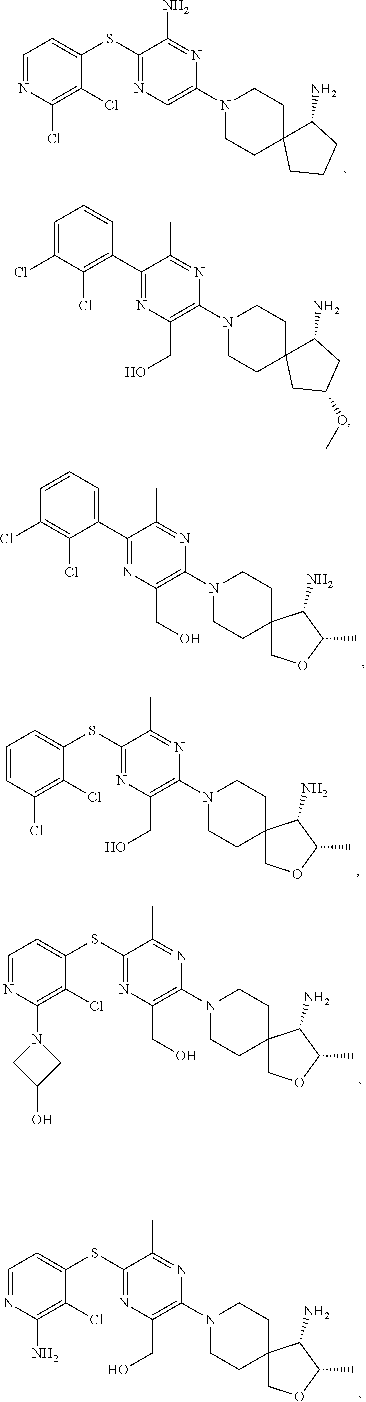 [SMILES: Nc1nc(N2CCC3(CCC[C@H]3N)CC2)cnc1Sc1ccnc(Cl)c1Cl ; CO[C@H]1C[C@@H](N)C2(CCN(c3nc(C)c(-c4cccc(Cl)c4Cl)nc3CO)CC2)C1 ; Cc1nc(N2CCC3(CC2)CO[C@@H](C)[C@H]3N)c(CO)nc1-c1cccc(Cl)c1Cl ; Cc1nc(N2CCC3(CC2)CO[C@@H](C)[C@H]3N)c(CO)nc1Sc1cccc(Cl)c1Cl ; Cc1nc(N2CCC3(CC2)CO[C@@H](C)[C@H]3N)c(CO)nc1Sc1ccnc(N2CC(O)C2)c1Cl ; Cc1nc(N2CCC3(CC2)CO[C@@H](C)[C@H]3N)c(CO)nc1Sc1ccnc(N)c1Cl]

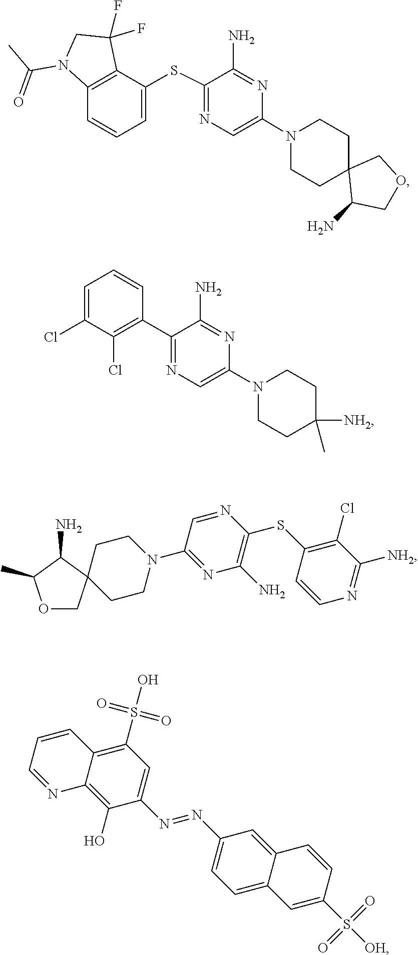 [SMILES: CC(=O)N1CC(F)(F)c2c(Sc3ncc(N4CCC5(CC4)COC[C@H]5N)nc3N)cccc21 ; CC1(N)CCN(c2cnc(-c3cccc(Cl)c3Cl)c(N)n2)CC1 ; C[C@@H]1OCC2(CCN(c3cnc(Sc4ccnc(N)c4Cl)c(N)n3)CC2)[C@@H]1N ; O=S(=O)(O)c1ccc2cc(/N=N/c3cc(S(=O)(=O)O)c4cccnc4c3O)ccc2c1]

or a pharmaceutically acceptable salt thereof.

In some embodiments, the neuroinflammation is associated with a separate disorder in the subject.

In one embodiment, described herein is a method of treating or preventing a neurodegenerative disorder in a subject in need thereof, comprising administering to the subject an effective amount of a SHP2 inhibitor having the structure:

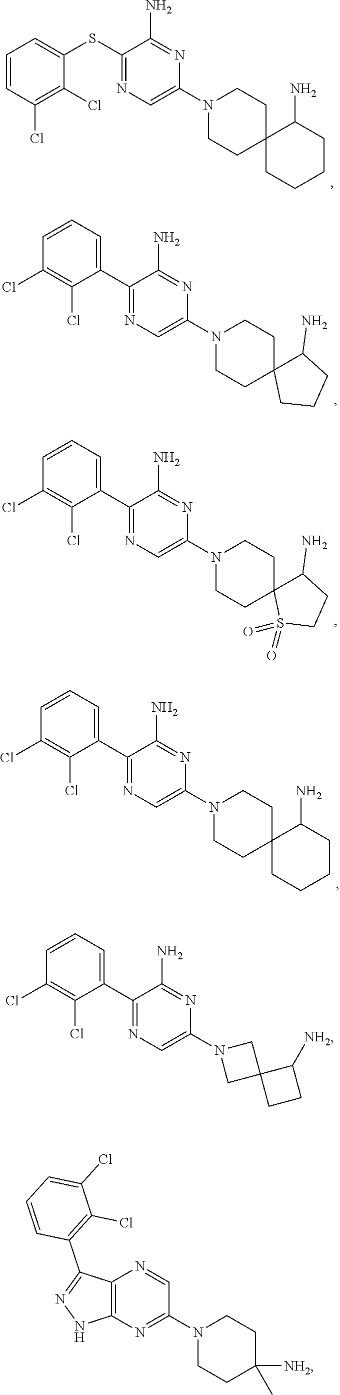 [SMILES: Nc1nc(N2CCC3(CCCCC3N)CC2)cnc1Sc1cccc(Cl)c1Cl ; Nc1nc(N2CCC3(CCCC3N)CC2)cnc1-c1cccc(Cl)c1Cl ; Nc1nc(N2CCC3(CC2)C(N)CCS3(=O)=O)cnc1-c1cccc(Cl)c1Cl ; Nc1nc(N2CCC3(CCCCC3N)CC2)cnc1-c1cccc(Cl)c1Cl ; Nc1nc(N2CC3(CCC3N)C2)cnc1-c1cccc(Cl)c1Cl ; CC1(N)CCN(c2cnc3c(-c4cccc(Cl)c4Cl)n[nH]c3n2)CC1]

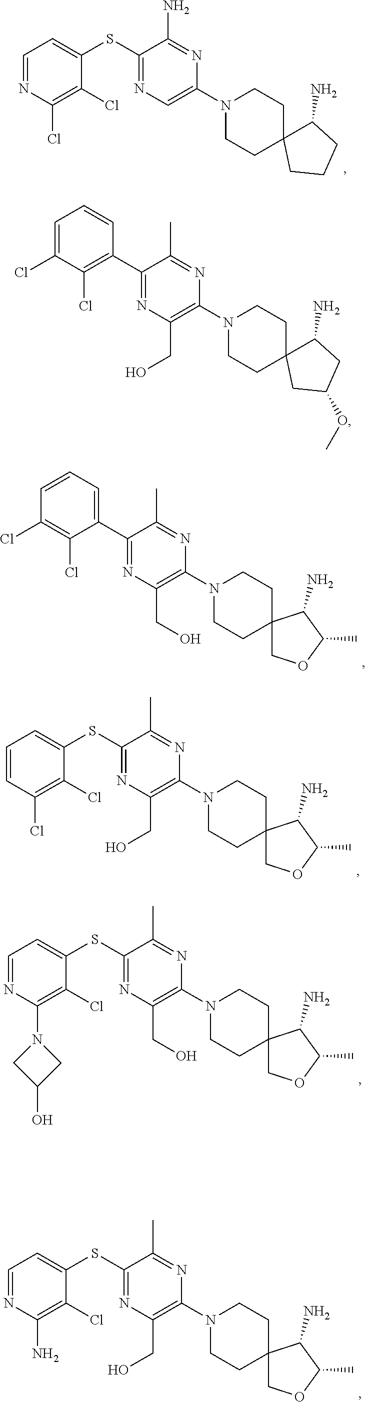 [SMILES: Nc1nc(N2CCC3(CCC[C@H]3N)CC2)cnc1Sc1ccnc(Cl)c1Cl ; CO[C@H]1C[C@@H](N)C2(CCN(c3nc(C)c(-c4cccc(Cl)c4Cl)nc3CO)CC2)C1 ; Cc1nc(N2CCC3(CC2)CO[C@@H](C)[C@H]3N)c(CO)nc1-c1cccc(Cl)c1Cl ; Cc1nc(N2CCC3(CC2)CO[C@@H](C)[C@H]3N)c(CO)nc1Sc1cccc(Cl)c1Cl ; Cc1nc(N2CCC3(CC2)CO[C@@H](C)[C@H]3N)c(CO)nc1Sc1ccnc(N2CC(O)C2)c1Cl ; Cc1nc(N2CCC3(CC2)CO[C@@H](C)[C@H]3N)c(CO)nc1Sc1ccnc(N)c1Cl]

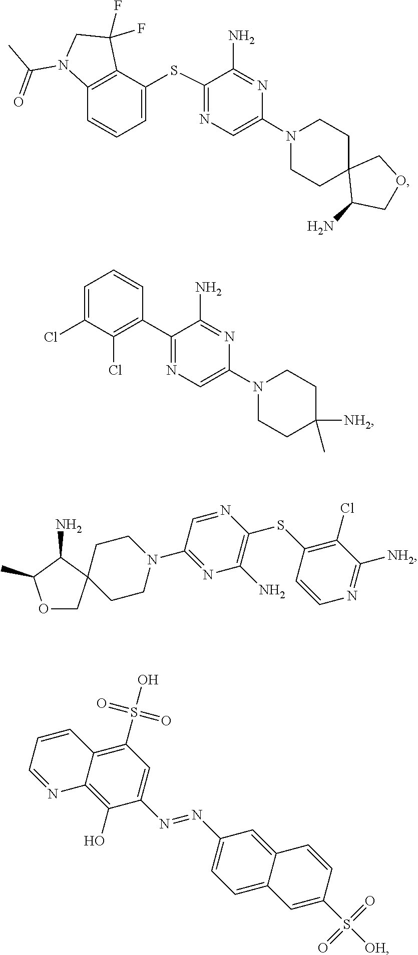 [SMILES: CC(=O)N1CC(F)(F)c2c(Sc3ncc(N4CCC5(CC4)COC[C@H]5N)nc3N)cccc21 ; CC1(N)CCN(c2cnc(-c3cccc(Cl)c3Cl)c(N)n2)CC1 ; C[C@@H]1OCC2(CCN(c3cnc(Sc4ccnc(N)c4Cl)c(N)n3)CC2)[C@@H]1N ; O=S(=O)(O)c1ccc2cc(/N=N/c3cc(S(=O)(=O)O)c4cccnc4c3O)ccc2c1]

or a pharmaceutically acceptable salt thereof.

In some embodiments, the neurodegenerative disorder is Alzheimer's disease, Parkinson's disease, dementia with Lewy bodies, multiple system atrophy, Huntington's disease, spinocerebellar ataxias, spinobulbar muscular atrophy (SBMA) or Kennedy disease, dentatorubropallidoluysian atrophy (DRPLA), ALS, AIDS dementia, frontotemporal dementia, corticobasal ganglionic degeneration, progressive supranuclear palsy, Creutzfeldt-Jakob disease, Gerstmann-Straussler-Scheinker syndrome, fatal familial insomnia, corticobasal ganglionic degeneration, hereditary spastic paraplegia, multiple sclerosis, neuromyelitis optica (Devic's disease), concentric sclerosis (Baló's disease), encephalomyelitis including acute disseminated encephalomyelitis (ADEM), acute haemorrhagic leucoencephalitis (AHL), Guillain-Barre Syndrome, chronic inflammatory demyelinating polyneuropathy (CIDP), transverse myelitis, Schilder's disease, fibromyalgia, or optic neuritis.

In one embodiment, described herein is a method of treating or preventing Multiple Sclerosis, Parkinson's disease, Multiple System Atrophy, Corticobasal Degeneration, Progressive Supranuclear Paresis, Guillain-Barre Syndrome (GBS), chronic inflammatory demyelinating polyneuropathy (CIDP), viral encephalitis, cerebrovascular accidents, or cranial trauma in a subject in need thereof, comprising administering to the subject an effective amount of a SHP2 inhibitor having the structure:

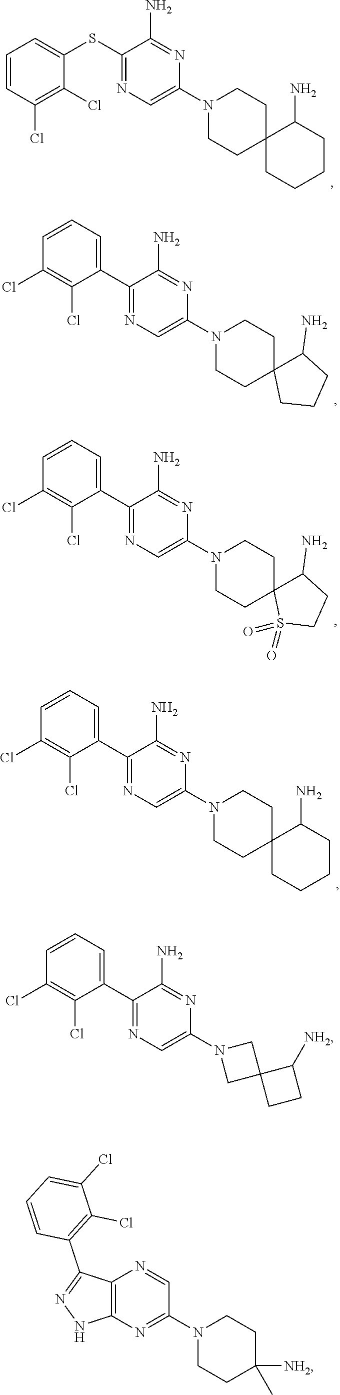 [SMILES: Nc1nc(N2CCC3(CCCCC3N)CC2)cnc1Sc1cccc(Cl)c1Cl ; Nc1nc(N2CCC3(CCCC3N)CC2)cnc1-c1cccc(Cl)c1Cl ; Nc1nc(N2CCC3(CC2)C(N)CCS3(=O)=O)cnc1-c1cccc(Cl)c1Cl ; Nc1nc(N2CCC3(CCCCC3N)CC2)cnc1-c1cccc(Cl)c1Cl ; Nc1nc(N2CC3(CCC3N)C2)cnc1-c1cccc(Cl)c1Cl ; CC1(N)CCN(c2cnc3c(-c4cccc(Cl)c4Cl)n[nH]c3n2)CC1]

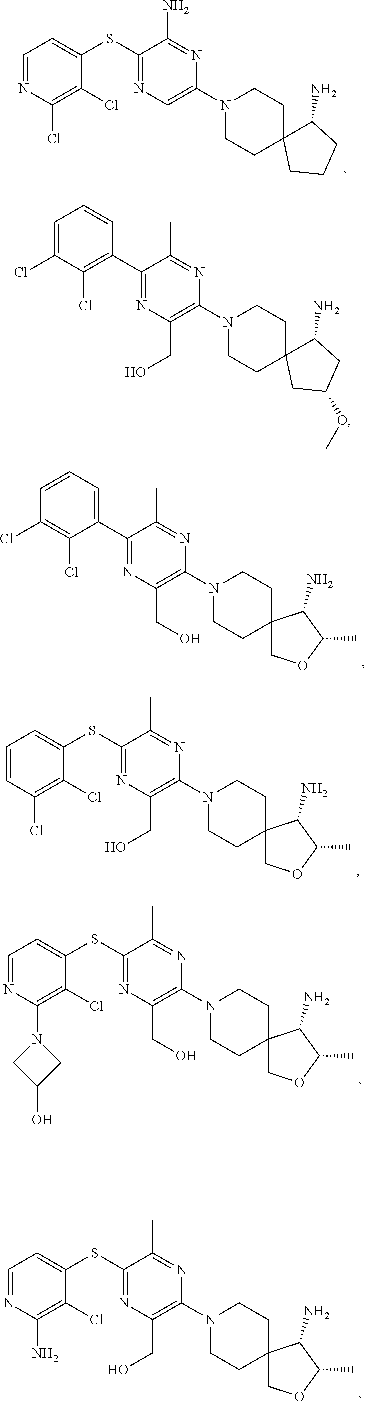 [SMILES: Nc1nc(N2CCC3(CCC[C@H]3N)CC2)cnc1Sc1ccnc(Cl)c1Cl ; CO[C@H]1C[C@@H](N)C2(CCN(c3nc(C)c(-c4cccc(Cl)c4Cl)nc3CO)CC2)C1 ; Cc1nc(N2CCC3(CC2)CO[C@@H](C)[C@H]3N)c(CO)nc1-c1cccc(Cl)c1Cl ; Cc1nc(N2CCC3(CC2)CO[C@@H](C)[C@H]3N)c(CO)nc1Sc1cccc(Cl)c1Cl ; Cc1nc(N2CCC3(CC2)CO[C@@H](C)[C@H]3N)c(CO)nc1Sc1ccnc(N2CC(O)C2)c1Cl ; Cc1nc(N2CCC3(CC2)CO[C@@H](C)[C@H]3N)c(CO)nc1Sc1ccnc(N)c1Cl]

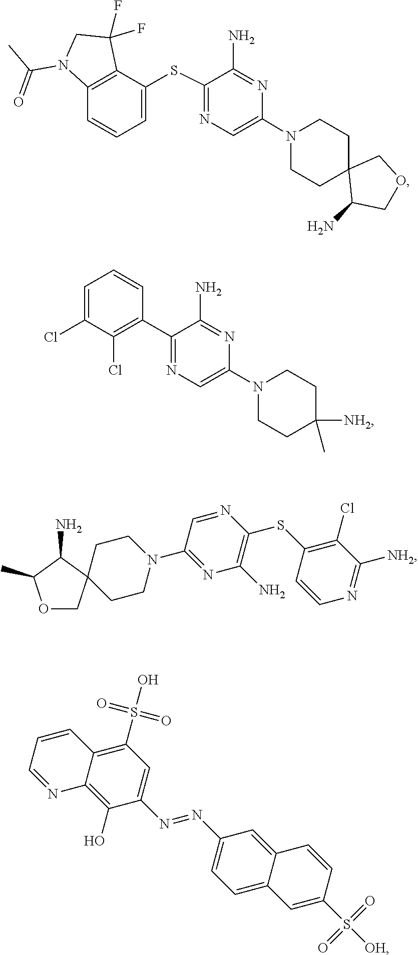 [SMILES: CC(=O)N1CC(F)(F)c2c(Sc3ncc(N4CCC5(CC4)COC[C@H]5N)nc3N)cccc21 ; CC1(N)CCN(c2cnc(-c3cccc(Cl)c3Cl)c(N)n2)CC1 ; C[C@@H]1OCC2(CCN(c3cnc(Sc4ccnc(N)c4Cl)c(N)n3)CC2)[C@@H]1N ; O=S(=O)(O)c1ccc2cc(/N=N/c3cc(S(=O)(=O)O)c4cccnc4c3O)ccc2c1]

or a pharmaceutically acceptable salt thereof.

In some embodiments, the Multiple Sclerosis is Relapse Remitting Multiple Sclerosis, Secondary Progressive Multiple Sclerosis or Primary Progressive Multiple Sclerosis.

In one embodiment, described herein method of treating or preventing a genetic disorder resulting in gain-of-function in SHP2 in a subject in need thereof, comprising administering to the subject an effective amount of a SHP2 inhibitor having the structure:

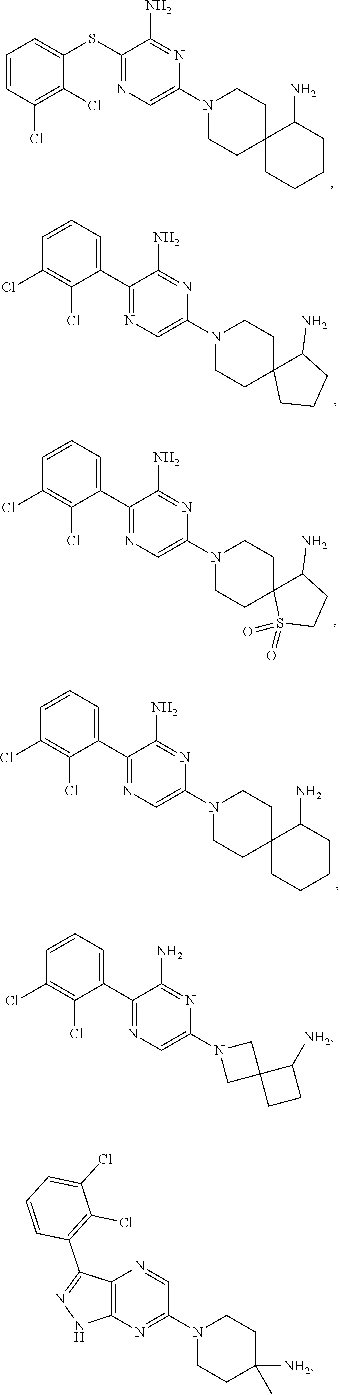 [SMILES: Nc1nc(N2CCC3(CCCCC3N)CC2)cnc1Sc1cccc(Cl)c1Cl ; Nc1nc(N2CCC3(CCCC3N)CC2)cnc1-c1cccc(Cl)c1Cl ; Nc1nc(N2CCC3(CC2)C(N)CCS3(=O)=O)cnc1-c1cccc(Cl)c1Cl ; Nc1nc(N2CCC3(CCCCC3N)CC2)cnc1-c1cccc(Cl)c1Cl ; Nc1nc(N2CC3(CCC3N)C2)cnc1-c1cccc(Cl)c1Cl ; CC1(N)CCN(c2cnc3c(-c4cccc(Cl)c4Cl)n[nH]c3n2)CC1]

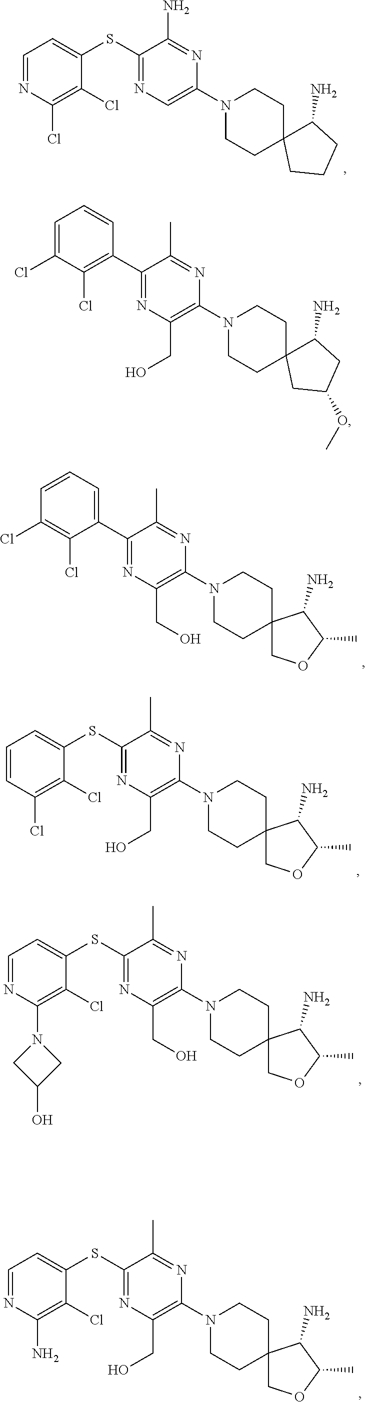 [SMILES: Nc1nc(N2CCC3(CCC[C@H]3N)CC2)cnc1Sc1ccnc(Cl)c1Cl ; CO[C@H]1C[C@@H](N)C2(CCN(c3nc(C)c(-c4cccc(Cl)c4Cl)nc3CO)CC2)C1 ; Cc1nc(N2CCC3(CC2)CO[C@@H](C)[C@H]3N)c(CO)nc1-c1cccc(Cl)c1Cl ; Cc1nc(N2CCC3(CC2)CO[C@@H](C)[C@H]3N)c(CO)nc1Sc1cccc(Cl)c1Cl ; Cc1nc(N2CCC3(CC2)CO[C@@H](C)[C@H]3N)c(CO)nc1Sc1ccnc(N2CC(O)C2)c1Cl ; Cc1nc(N2CCC3(CC2)CO[C@@H](C)[C@H]3N)c(CO)nc1Sc1ccnc(N)c1Cl]

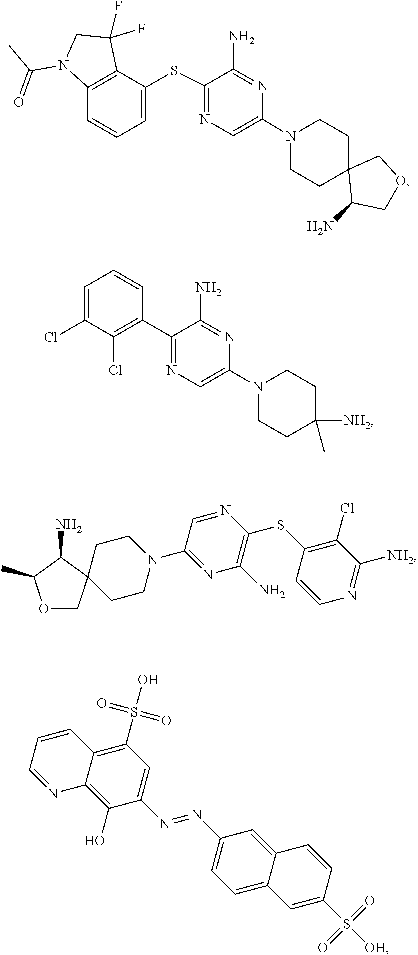 [SMILES: CC(=O)N1CC(F)(F)c2c(Sc3ncc(N4CCC5(CC4)COC[C@H]5N)nc3N)cccc21 ; CC1(N)CCN(c2cnc(-c3cccc(Cl)c3Cl)c(N)n2)CC1 ; C[C@@H]1OCC2(CCN(c3cnc(Sc4ccnc(N)c4Cl)c(N)n3)CC2)[C@@H]1N ; O=S(=O)(O)c1ccc2cc(/N=N/c3cc(S(=O)(=O)O)c4cccnc4c3O)ccc2c1]

or a pharmaceutically acceptable salt thereof.

In some embodiments, the genetic disorder resulting in gain-of-function in SHP2 is Noonan syndrome or Leopard syndrome.

In one embodiment, described herein is a method of treating or preventing a genetic disorder resulting in loss-of-function in the ras signaling pathway in a subject in need thereof, comprising administering to the subject an effective amount of a SHP2 inhibitor having the structure:

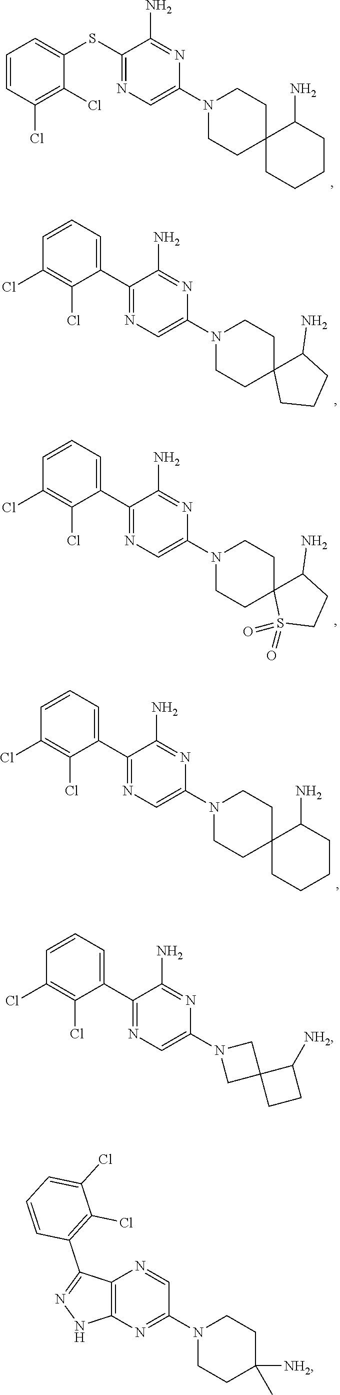 [SMILES: Nc1nc(N2CCC3(CCCCC3N)CC2)cnc1Sc1cccc(Cl)c1Cl ; Nc1nc(N2CCC3(CCCC3N)CC2)cnc1-c1cccc(Cl)c1Cl ; Nc1nc(N2CCC3(CC2)C(N)CCS3(=O)=O)cnc1-c1cccc(Cl)c1Cl ; Nc1nc(N2CCC3(CCCCC3N)CC2)cnc1-c1cccc(Cl)c1Cl ; Nc1nc(N2CC3(CCC3N)C2)cnc1-c1cccc(Cl)c1Cl ; CC1(N)CCN(c2cnc3c(-c4cccc(Cl)c4Cl)n[nH]c3n2)CC1]

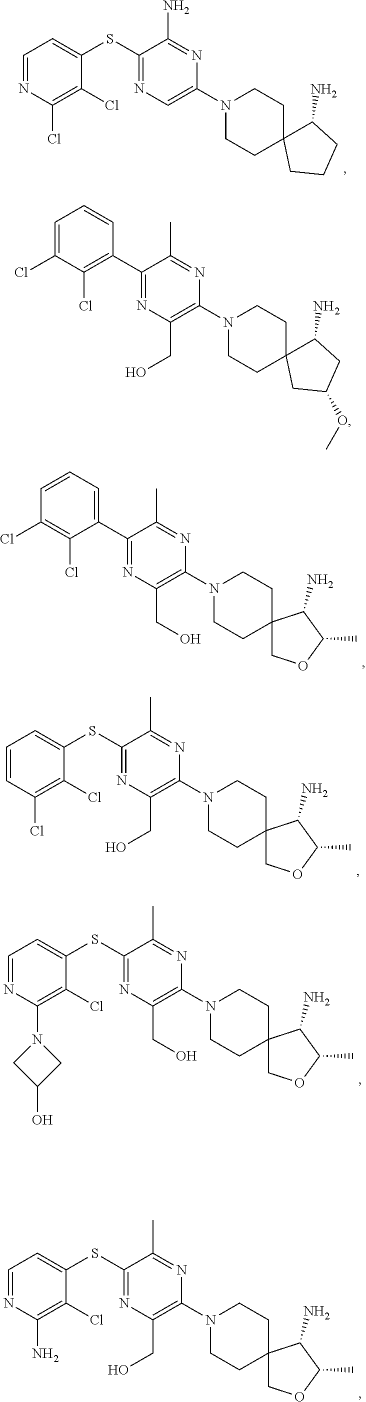 [SMILES: Nc1nc(N2CCC3(CCC[C@H]3N)CC2)cnc1Sc1ccnc(Cl)c1Cl ; CO[C@H]1C[C@@H](N)C2(CCN(c3nc(C)c(-c4cccc(Cl)c4Cl)nc3CO)CC2)C1 ; Cc1nc(N2CCC3(CC2)CO[C@@H](C)[C@H]3N)c(CO)nc1-c1cccc(Cl)c1Cl ; Cc1nc(N2CCC3(CC2)CO[C@@H](C)[C@H]3N)c(CO)nc1Sc1cccc(Cl)c1Cl ; Cc1nc(N2CCC3(CC2)CO[C@@H](C)[C@H]3N)c(CO)nc1Sc1ccnc(N2CC(O)C2)c1Cl ; Cc1nc(N2CCC3(CC2)CO[C@@H](C)[C@H]3N)c(CO)nc1Sc1ccnc(N)c1Cl]

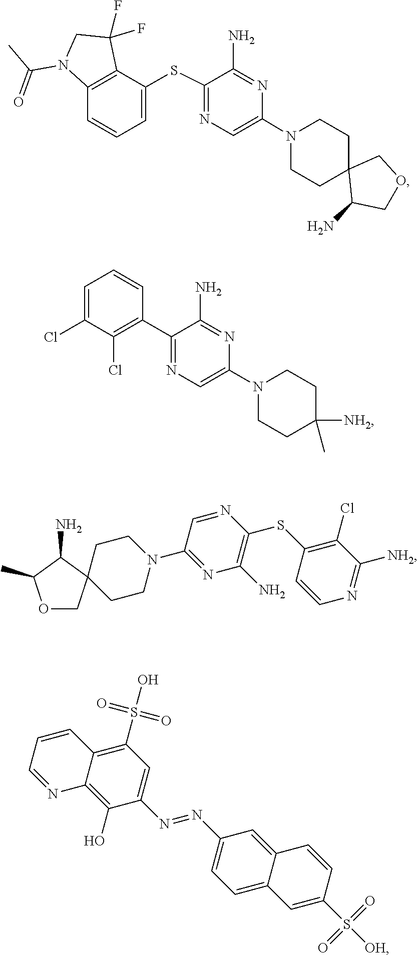 [SMILES: CC(=O)N1CC(F)(F)c2c(Sc3ncc(N4CCC5(CC4)COC[C@H]5N)nc3N)cccc21 ; CC1(N)CCN(c2cnc(-c3cccc(Cl)c3Cl)c(N)n2)CC1 ; C[C@@H]1OCC2(CCN(c3cnc(Sc4ccnc(N)c4Cl)c(N)n3)CC2)[C@@H]1N ; O=S(=O)(O)c1ccc2cc(/N=N/c3cc(S(=O)(=O)O)c4cccnc4c3O)ccc2c1]

or a pharmaceutically acceptable salt thereof.

In some embodiments, the genetic disorder resulting in loss-of-function in the ras signaling pathway is Legius syndrome.

In an embodiment, described herein is a method of treating or preventing demyelination in a subject in need thereof, comprising administering to the subject an effective amount of a SHP2 inhibitor having the structure:

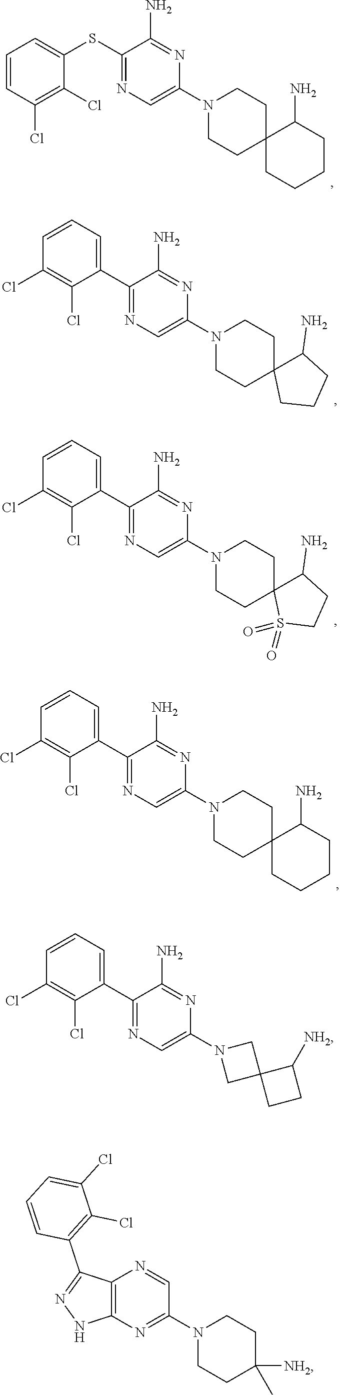 [SMILES: Nc1nc(N2CCC3(CCCCC3N)CC2)cnc1Sc1cccc(Cl)c1Cl ; Nc1nc(N2CCC3(CCCC3N)CC2)cnc1-c1cccc(Cl)c1Cl ; Nc1nc(N2CCC3(CC2)C(N)CCS3(=O)=O)cnc1-c1cccc(Cl)c1Cl ; Nc1nc(N2CCC3(CCCCC3N)CC2)cnc1-c1cccc(Cl)c1Cl ; Nc1nc(N2CC3(CCC3N)C2)cnc1-c1cccc(Cl)c1Cl ; CC1(N)CCN(c2cnc3c(-c4cccc(Cl)c4Cl)n[nH]c3n2)CC1]

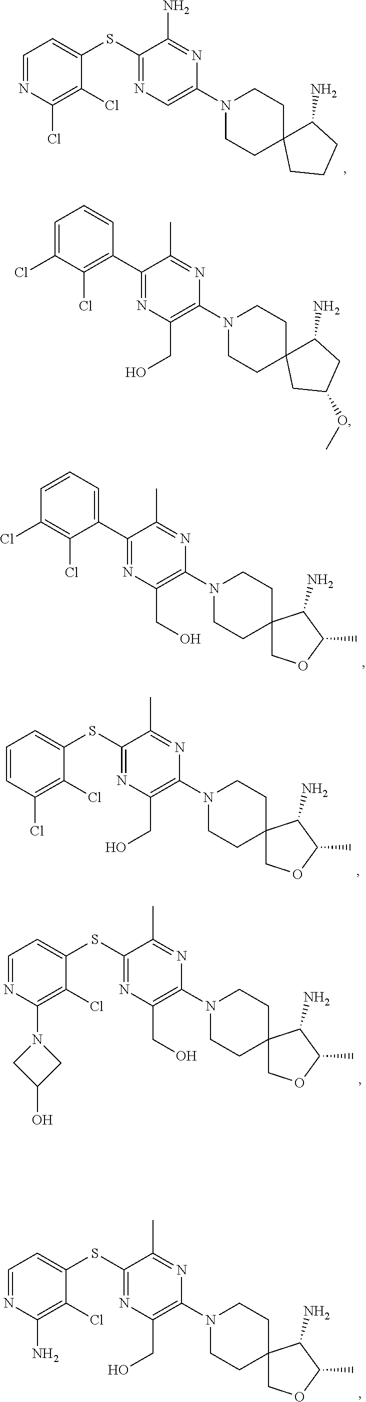 [SMILES: Nc1nc(N2CCC3(CCC[C@H]3N)CC2)cnc1Sc1ccnc(Cl)c1Cl ; CO[C@H]1C[C@@H](N)C2(CCN(c3nc(C)c(-c4cccc(Cl)c4Cl)nc3CO)CC2)C1 ; Cc1nc(N2CCC3(CC2)CO[C@@H](C)[C@H]3N)c(CO)nc1-c1cccc(Cl)c1Cl ; Cc1nc(N2CCC3(CC2)CO[C@@H](C)[C@H]3N)c(CO)nc1Sc1cccc(Cl)c1Cl ; Cc1nc(N2CCC3(CC2)CO[C@@H](C)[C@H]3N)c(CO)nc1Sc1ccnc(N2CC(O)C2)c1Cl ; Cc1nc(N2CCC3(CC2)CO[C@@H](C)[C@H]3N)c(CO)nc1Sc1ccnc(N)c1Cl]

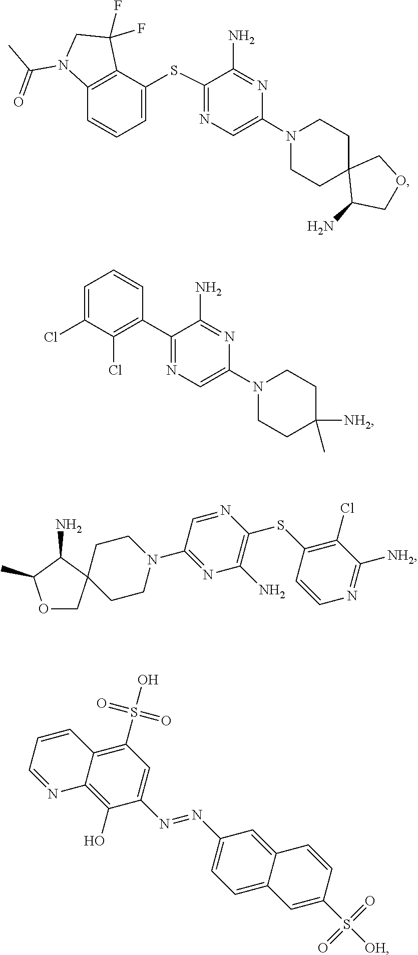 [SMILES: CC(=O)N1CC(F)(F)c2c(Sc3ncc(N4CCC5(CC4)COC[C@H]5N)nc3N)cccc21 ; CC1(N)CCN(c2cnc(-c3cccc(Cl)c3Cl)c(N)n2)CC1 ; C[C@@H]1OCC2(CCN(c3cnc(Sc4ccnc(N)c4Cl)c(N)n3)CC2)[C@@H]1N ; O=S(=O)(O)c1ccc2cc(/N=N/c3cc(S(=O)(=O)O)c4cccnc4c3O)ccc2c1]

or a pharmaceutically acceptable salt thereof.

In some embodiments, the demyelation is caused by an injury, a hypoxic-ischaemic event, a metabolic disruption, an inherited condition, or an exposure to a toxic substance. In some embodiments, the injury is a spinal cord injury, traumatic brain injury, cerebral palsy, or neuropathy. In some embodiments, the hypoxic-ischaemic event is stroke, acute ischemic optic neuropathy, or other ischemia, or carbon monoxide exposure. In some embodiments, the metabolic disruption is entral pontine myelolysis (CPM), or extrapontine myelinolysis (EPM). In some embodiments, the inherited condition is Charcot-Marie-Tooth disease (CMT), Sjogren-Larsson syndrome, Refsum disease, Krabbe disease, Canavan disease, Alexander disease, Friedreich's ataxia, Pelizaeus-Merzbacher disease, Bassen-Kornzweig syndrome, metachromatic leukodystrophy (MLD), adrenoleukodystrophy, Leber's optic neuropathy, or nerve damage due to pernicious anemia. In some embodiments, the exposure to a toxic substance is chronic alcoholism.

In an embodiment, described herein is a method of treating or preventing Noonan syndrome, Leopard syndrome, Legius syndrome, Alzheimer's disease, Parkinson's disease, dementia with Lewy bodies, multiple system atrophy, Huntington's disease, spinocerebellar ataxias (e.g., SCA1, SCA2, SCA3, SCA6, SCAT, and SCA17), spinobulbar muscular atrophy (SBMA), Kennedy disease, dentatorubropallidoluysian atrophy (DRPLA), ALS, AIDS dementia, frontotemporal dementia, corticobasal ganglionic degeneration, progressive supranuclear palsy, Creutzfeldt-Jakob disease, Gerstmann-Straussler-Scheinker syndrome, fatal familial insomnia, corticobasal ganglionic degeneration, hereditary spastic paraplegia, multiple sclerosis, neuromyelitis optica (Devic's disease), concentric sclerosis (Baló's disease), acute disseminated encephalomyelitis (ADEM), acute haemorrhagic leucoencephalitis (AHL), Guillain-Barre Syndrome, chronic inflammatory demyelinating polyneuropathy (CIDP), transverse myelitis, Schilder's disease, fibromyalgia, optic neuritis, spinal cord injury, traumatic brain injury, cerebral palsy, neuropathy; stroke, acute ischemic optic neuropathy, or other ischemia, and carbon monoxide exposure; central pontine myelolysis (CPM), extrapontine myelinolysis (EPM), Charcot-Marie-Tooth disease (CMT), Sjogren-Larsson syndrome, Refsum disease, Krabbe disease, Canavan disease, Alexander disease, Friedreich's ataxia, Pelizaeus-Merzbacher disease, Bassen-Kornzweig syndrome, metachromatic leukodystrophy (MLD), adrenoleukodystrophy, Leber's optic neuropathy, nerve damage due to pernicious anemia, progressive multifocal leukoencephalopathy (PML), Lyme disease, tabes dorsalis due to untreated syphilis, HIV, subacute sclerosing panencephalitis due to measles virus, Marchiafava-Bignami disease, chemotherapy, a disorder resulting from exposure to mitochondrial toxins, or exposure to chemicals, vitamin B12 deficiency, vitamin E deficiency, copper deficiency, trigeminal neuralgia, Marchiafava-Bignami disease, or Bell's palsy in a subject in need thereof, comprising administering to the subject an effective amount of a SHP2 inhibitor having the structure:

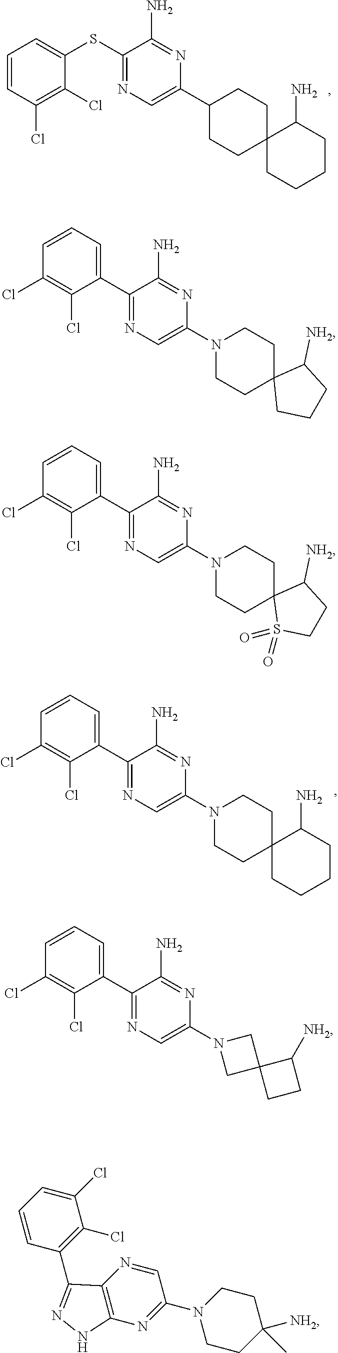 [SMILES: Nc1nc(C2CCC3(CCCCC3N)CC2)cnc1Sc1cccc(Cl)c1Cl ; Nc1nc(N2CCC3(CCCC3N)CC2)cnc1-c1cccc(Cl)c1Cl ; Nc1nc(N2CCC3(CC2)C(N)CCS3(=O)=O)cnc1-c1cccc(Cl)c1Cl ; Nc1nc(N2CCC3(CCCCC3N)CC2)cnc1-c1cccc(Cl)c1Cl ; Nc1nc(N2CC3(CCC3N)C2)cnc1-c1cccc(Cl)c1Cl ; CC1(N)CCN(c2cnc3c(-c4cccc(Cl)c4Cl)n[nH]c3n2)CC1]

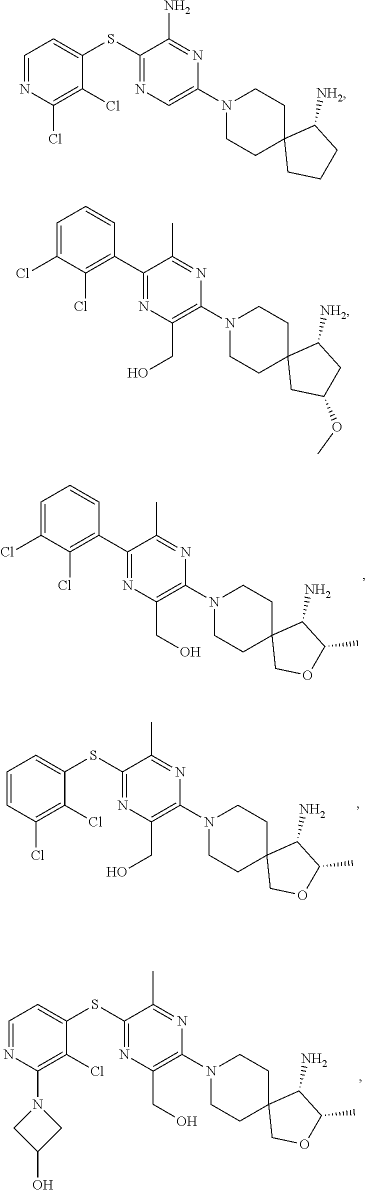 [SMILES: Nc1nc(N2CCC3(CCC[C@H]3N)CC2)cnc1Sc1ccnc(Cl)c1Cl ; CO[C@H]1C[C@@H](N)C2(CCN(c3nc(C)c(-c4cccc(Cl)c4Cl)nc3CO)CC2)C1 ; Cc1nc(N2CCC3(CC2)CO[C@@H](C)[C@H]3N)c(CO)nc1-c1cccc(Cl)c1Cl ; Cc1nc(N2CCC3(CC2)CO[C@@H](C)[C@H]3N)c(CO)nc1Sc1cccc(Cl)c1Cl ; Cc1nc(N2CCC3(CC2)CO[C@@H](C)[C@H]3N)c(CO)nc1Sc1ccnc(N2CC(O)C2)c1Cl]

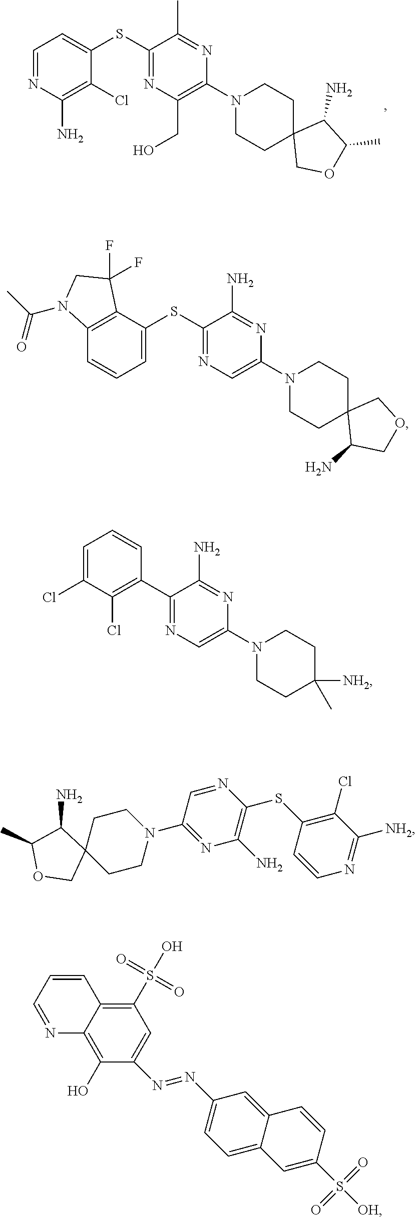

or a pharmaceutically acceptable salt thereof. In some embodiments, the neuropathy is neuropathy due to diabetes, chronic renal failure, hypothyroidism, liver failure, or compression of the nerve (e.g. in Bell's palsy), or post radiation injury.

In some embodiments, the SHP2 inhibitor is:

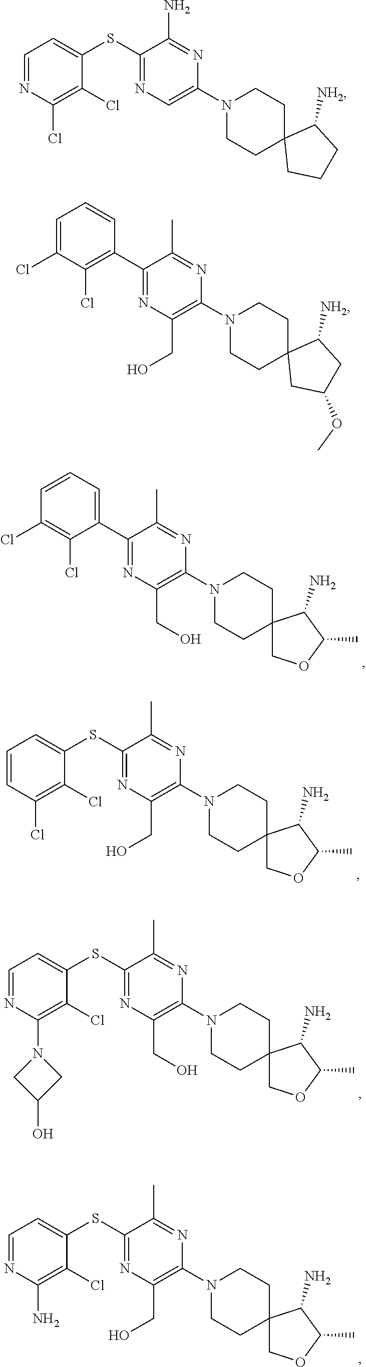 [SMILES: Nc1nc(N2CCC3(CCC[C@H]3N)CC2)cnc1Sc1ccnc(Cl)c1Cl ; CO[C@H]1C[C@@H](N)C2(CCN(c3nc(C)c(-c4cccc(Cl)c4Cl)nc3CO)CC2)C1 ; Cc1nc(N2CCC3(CC2)CO[C@@H](C)[C@H]3N)c(CO)nc1-c1cccc(Cl)c1Cl ; Cc1nc(N2CCC3(CC2)CO[C@@H](C)[C@H]3N)c(CO)nc1Sc1cccc(Cl)c1Cl ; Cc1nc(N2CCC3(CC2)CO[C@@H](C)[C@H]3N)c(CO)nc1Sc1ccnc(N2CC(O)C2)c1Cl ; Cc1nc(N2CCC3(CC2)CO[C@@H](C)[C@H]3N)c(CO)nc1Sc1ccnc(N)c1Cl]

or a pharmaceutically acceptable salt thereof. In some embodiments, the SHP2 inhibitor is:

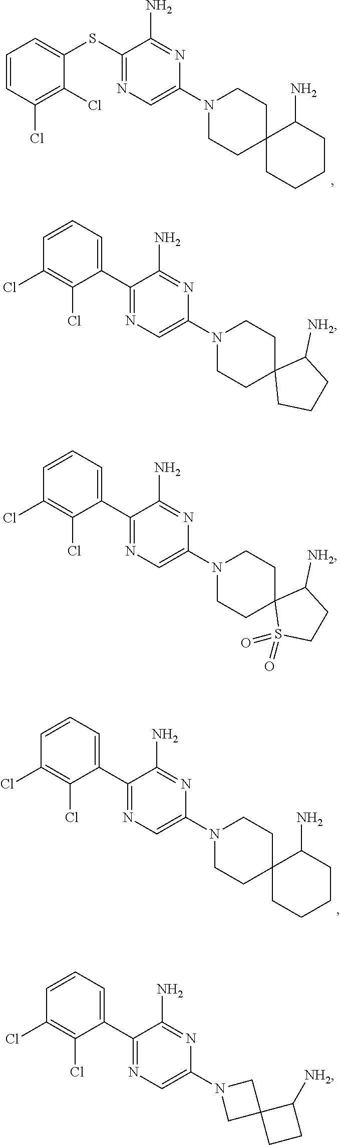 [SMILES: Nc1nc(N2CCC3(CCCCC3N)CC2)cnc1Sc1cccc(Cl)c1Cl ; Nc1nc(N2CCC3(CCCC3N)CC2)cnc1-c1cccc(Cl)c1Cl ; Nc1nc(N2CCC3(CC2)C(N)CCS3(=O)=O)cnc1-c1cccc(Cl)c1Cl ; Nc1nc(N2CCC3(CCCCC3N)CC2)cnc1-c1cccc(Cl)c1Cl ; Nc1nc(N2CC3(CCC3N)C2)cnc1-c1cccc(Cl)c1Cl]

or a pharmaceutically acceptable salt thereof. In some embodiments, the SHP2 inhibitor is:

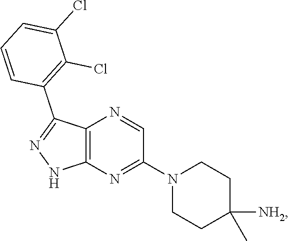 [SMILES: CC1(N)CCN(c2cnc3c(-c4cccc(Cl)c4Cl)n[nH]c3n2)CC1]

or a pharmaceutically acceptable salt thereof. In some embodiments, the SHP2 inhibitor is

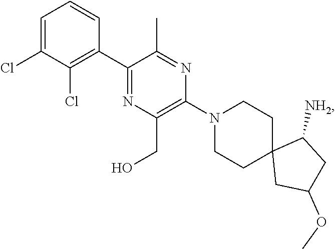 [SMILES: COC1C[C@@H](N)C2(CCN(c3nc(C)c(-c4cccc(Cl)c4Cl)nc3CO)CC2)C1]

or a pharmaceutically acceptable salt thereof. In some embodiments, the SHP2 inhibitor is

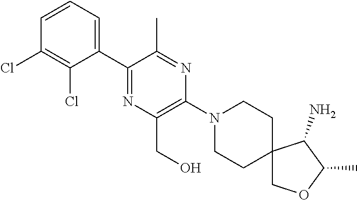 [SMILES: Cc1nc(N2CCC3(CC2)CO[C@@H](C)[C@H]3N)c(CO)nc1-c1cccc(Cl)c1Cl]

or a pharmaceutically acceptable salt thereof. In some embodiments, the SHP2 inhibitor is

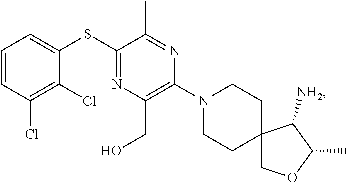 [SMILES: Cc1nc(N2CCC3(CC2)CO[C@@H](C)[C@H]3N)c(CO)nc1Sc1cccc(Cl)c1Cl]

or a pharmaceutically acceptable salt thereof. In some embodiments, the SHP2 inhibitor is

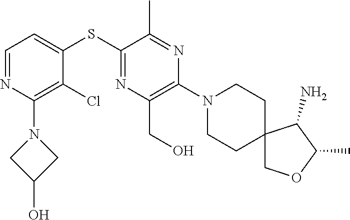 [SMILES: Cc1nc(N2CCC3(CC2)CO[C@@H](C)[C@H]3N)c(CO)nc1Sc1ccnc(N2CC(O)C2)c1Cl]

or a pharmaceutically acceptable salt thereof. In some embodiments, the SHP2 inhibitor is

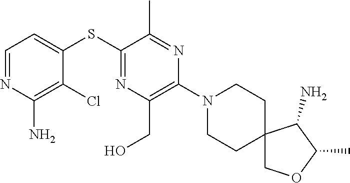 [SMILES: Cc1nc(N2CCC3(CC2)CO[C@@H](C)[C@H]3N)c(CO)nc1Sc1ccnc(N)c1Cl]

or a pharmaceutically acceptable salt thereof. In some embodiments, the SHP2 inhibitor is

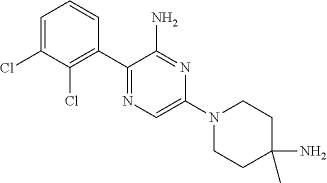 [SMILES: CC1(N)CCN(c2cnc(-c3cccc(Cl)c3Cl)c(N)n2)CC1]

or a pharmaceutically acceptable salt thereof. In some embodiments, the SHP2 inhibitor is

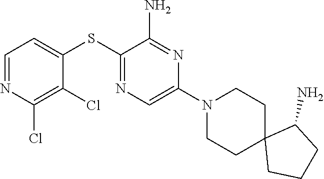 [SMILES: Nc1nc(N2CCC3(CCC[C@H]3N)CC2)cnc1Sc1ccnc(Cl)c1Cl]

or a pharmaceutically acceptable salt thereof. In some embodiments, the SHP2 inhibitor is

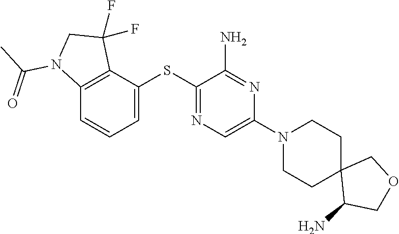 [SMILES: CC(=O)N1CC(F)(F)c2c(Sc3ncc(N4CCC5(CC4)COC[C@H]5N)nc3N)cccc21]

or a pharmaceutically acceptable salt thereof. In some embodiments, the SHP2 inhibitor is

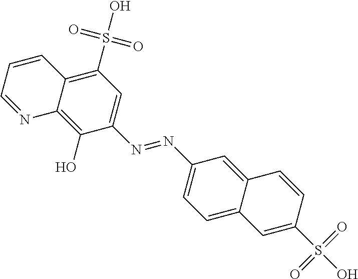 [SMILES: O=S(=O)(O)c1ccc2cc(/N=N/c3cc(S(=O)(=O)O)c4cccnc4c3O)ccc2c1]

or a pharmaceutically acceptable salt thereof. In some embodiments, the SHP2 inhibitor is administered orally, parenterally, rectally, transdermally, intradermally, intrathecally, subcutaneously, intravenously, intramuscularly, or intranasally. In some embodiments, the SHP2 inhibitor is orally administered.

In an embodiment, described herein is a method of treating or preventing neuroinflammation in a subject in need thereof, the method comprising administering to the subject an effective amount of a SHP1 inhibitor.

In an embodiment, described herein is a method of treating or preventing a neuroinflammatory disorder in a subject in need thereof, the method comprising administering to the subject an effective amount of a SHP1 inhibitor.

In an embodiment, described herein is a method of treating or preventing a disorder associated with neuroinflammation in a subject in need thereof, the method comprising administering to the subject an effective amount of a SHP1 inhibitor.

In some embodiments, the neuroinflammation is associated with a separate disorder in the subject.

In an embodiment, described herein is a method of treating or preventing a neurodegenerative disorder in a subject in need thereof, comprising administering to the subject an effective amount of a SHP1 inhibitor.

In some embodiments, the neurodegenerative disorder is Alzheimer's disease, Parkinson's disease, dementia with Lewy bodies, multiple system atrophy, Huntington's disease, spinocerebellar ataxias, spinobulbar muscular atrophy (SBMA) or Kennedy disease, dentatorubropallidoluysian atrophy (DRPLA), ALS, AIDS dementia, frontotemporal dementia, corticobasal ganglionic degeneration, progressive supranuclear palsy, Creutzfeldt-Jakob disease, Gerstmann-Straussler-Scheinker syndrome, fatal familial insomnia, corticobasal ganglionic degeneration, hereditary spastic paraplegia, multiple sclerosis, neuromyelitis optica (Devic's disease), concentric sclerosis (Baló's disease), encephalomyelitis including acute disseminated encephalomyelitis (ADEM), acute haemorrhagic leucoencephalitis (AHL), Guillain-Barre Syndrome, chronic inflammatory demyelinating polyneuropathy (CIDP), transverse myelitis, Schilder's disease, fibromyalgia, or optic neuritis.

In an embodiment, described herein is a method of treating or preventing Multiple Sclerosis, Parkinson's disease, Multiple System Atrophy, Corticobasal Degeneration, Progressive Supranuclear Paresis, Guillain-Barre Syndrome (GBS), chronic inflammatory demyelinating polyneuropathy (CIDP), viral encephalitis, cerebrovascular accidents, or cranial trauma in a subject in need thereof, comprising administering to the subject an effective amount of a SHP1 inhibitor.

In some embodiments, the Multiple Sclerosis is Relapse Remitting Multiple Sclerosis, Secondary Progressive Multiple Sclerosis or Primary Progressive Multiple Sclerosis.

In an embodiment, described herein is a method of treating or preventing a genetic disorder resulting in loss-of-function in the ras signaling pathway in a subject in need thereof, comprising administering to the subject an effective amount of a SHP1 inhibitor.

In some embodiments, the genetic disorder resulting in loss-of-function in the ras signaling pathway is Legius syndrome.

In an embodiment, described herein is a method of treating or preventing demyelination in a subject in need thereof, comprising administering to the subject an effective amount of a SHP1 inhibitor.

In some embodiments, the demyelation is caused by an injury, a hypoxic-ischaemic event, a metabolic disruption, an inherited condition, or an exposure to a toxic substance.

In some embodiments, the injury is a spinal cord injury, traumatic brain injury, cerebral palsy, or neuropathy.

In some embodiments, the hypoxic-ischaemic event is stroke, acute ischemic optic neuropathy, or other ischemia, or carbon monoxide exposure.

In some embodiments, the metabolic disruption is entral pontine myelolysis (CPM), or extrapontine myelinolysis (EPM).

In some embodiments, the inherited condition is Charcot-Marie-Tooth disease (CMT), Sjogren-Larsson syndrome, Refsum disease, Krabbe disease, Canavan disease, Alexander disease, Friedreich's ataxia, Pelizaeus-Merzbacher disease, Bassen-Kornzweig syndrome, metachromatic leukodystrophy (MLD), adrenoleukodystrophy, Leber's optic neuropathy, or nerve damage due to pernicious anemia.

In some embodiments, the exposure to a toxic substance is chronic alcoholism.

In an embodiment, described herein is a method of treating or preventing Noonan syndrome, Leopard syndrome, Legius syndrome, Alzheimer's disease, Parkinson's disease, dementia with Lewy bodies, multiple system atrophy, Huntington's disease, spinocerebellar ataxias (e.g., SCA1, SCA2, SCA3, SCA6, SCAT, and SCA17), spinobulbar muscular atrophy (SBMA), Kennedy disease, dentatorubropallidoluysian atrophy (DRPLA), ALS, AIDS dementia, frontotemporal dementia, corticobasal ganglionic degeneration, progressive supranuclear palsy, Creutzfeldt-Jakob disease, Gerstmann-Straussler-Scheinker syndrome, fatal familial insomnia, corticobasal ganglionic degeneration, hereditary spastic paraplegia, multiple sclerosis, neuromyelitis optica (Devic's disease), concentric sclerosis (Baló's disease), acute disseminated encephalomyelitis (ADEM), acute haemorrhagic leucoencephalitis (AHL), Guillain-Barre Syndrome, chronic inflammatory demyelinating polyneuropathy (CIDP), transverse myelitis, Schilder's disease, fibromyalgia, optic neuritis, spinal cord injury, traumatic brain injury, cerebral palsy, neuropathy; stroke, acute ischemic optic neuropathy, or other ischemia, and carbon monoxide exposure; central pontine myelolysis (CPM), extrapontine myelinolysis (EPM), Charcot-Marie-Tooth disease (CMT), Sjogren-Larsson syndrome, Refsum disease, Krabbe disease, Canavan disease, Alexander disease, Friedreich's ataxia, Pelizaeus-Merzbacher disease, Bassen-Kornzweig syndrome, metachromatic leukodystrophy (MLD), adrenoleukodystrophy, Leber's optic neuropathy, nerve damage due to pernicious anemia, progressive multifocal leukoencephalopathy (PML), Lyme disease, tabes dorsalis due to untreated syphilis, HIV, subacute sclerosing panencephalitis due to measles virus, Marchiafava-Bignami disease, chemotherapy, a disorder resulting from exposure to mitochondrial toxins, or exposure to chemicals, vitamin B12 deficiency, vitamin E deficiency, copper deficiency, trigeminal neuralgia, Marchiafava-Bignami disease, or Bell's palsy in a subject in need thereof, comprising administering to the subject an effective amount of a SHP1 inhibitor.

In some embodiments, the SHP1 inhibitor is administered orally, parenterally, rectally, transdermally, intradermally, intrathecally, subcutaneously, intravenously, intramuscularly, or intranasally.

In some embodiments, the SHP1 inhibitor is orally administered.

Also described herein is a method of treating or preventing a neuroinflammatory disorder in a subject in need thereof, the method comprising administering to the subject an effective amount of a compound described herein, e.g., a SHP1 inhibitor described herein or a SHP2 inhibitor described herein, or pharmaceutical composition thereof.

Also described herein is a method of treating or preventing a disorder associated with neuroinflammation in a subject in need thereof, the method comprising administering to the subject an effective amount of a compound described herein, e.g., a SHP1 inhibitor described herein or a SHP2 inhibitor described herein, or pharmaceutical composition thereof.

Also described herein is a method of treating or preventing Multiple Sclerosis, Parkinson's disease, Multiple System Atrophy, Corticobasal Degeneration, Progressive Supranuclear Paresis, Guillain-Barre Syndrome (GBS), chronic inflammatory demyelinating polyneuropathy (CIDP), viral encephalitis, cerebrovascular accidents, or cranial trauma in a subject in need thereof, comprising administering to the subject an effective amount of a compound described herein, e.g., a SHP1 inhibitor described herein or a SHP2 inhibitor described herein, or pharmaceutical composition thereof.

Additionally described herein is a method of treating or preventing genetic disorders, e.g., genetic disorders resulting in gain-of-function in SHP2, such as Noonan syndrome and Leopard syndrome, genetic disorders resulting in loss-of-function in the ras signaling pathway including Legius syndrome, neurodegenerative diseases such as Alzheimer's disease, Parkinson's disease, dementia with Lewy bodies, multiple system atrophy, Huntington's disease, spinocerebellar ataxias (e.g., SCA1, SCA2, SCA3, SCA6, SCAT, and SCA17), spinobulbar muscular atrophy (SBMA) or Kennedy disease, dentatorubropallidoluysian atrophy (DRPLA), ALS, AIDS dementia, frontotemporal dementia, corticobasal ganglionic degeneration, progressive supranuclear palsy, Creutzfeldt-Jakob disease, Gerstmann-Straussler-Scheinker syndrome, fatal familial insomnia, corticobasal ganglionic degeneration, hereditary spastic paraplegia, multiple sclerosis, neuromyelitis optica (Devic's disease), concentric sclerosis (Baló's disease), encephalomyelitis including acute disseminated encephalomyelitis (ADEM), acute haemorrhagic leucoencephalitis (AHL), Guillain-Barre Syndrome, chronic inflammatory demyelinating polyneuropathy (CIDP), transverse myelitis, Schilder's disease, fibromyalgia, and optic neuritis; demyelination due to injury such as spinal cord injury, traumatic brain injury, cerebral palsy, neuropathy (e.g. neuropathy due to diabetes, chronic renal failure, hypothyroidism, liver failure, or compression of the nerve (e.g. in Bell's palsy) and post radiation injury; demyelination due to hypoxic-ischaemic events such as stroke, acute ischemic optic neuropathy, or other ischemia, and carbon monoxide exposure; demyelination due to metabolic disruption such as central pontine myelolysis (CPM) or extrapontine myelinolysis (EPM); demyelination due to inherited conditions such as Charcot-Marie-Tooth disease (CMT), Sjogren-Larsson syndrome, Refsum disease, Krabbe disease, Canavan disease, Alexander disease, Friedreich's ataxia, Pelizaeus-Merzbacher disease, Bassen-Kornzweig syndrome, metachromatic leukodystrophy (MLD), adrenoleukodystrophy, Leber's optic neuropathy, and nerve damage due to pernicious anemia; demyelination due to a viral infection such as progressive multifocal leukoencephalopathy (PML), Lyme disease, tabes dorsalis due to untreated syphilis, HIV, or subacute sclerosing panencephalitis due to measles virus; demyelination due to toxic exposure such as chronic alcoholism (which is a possible cause of Marchiafava-Bignami disease), chemotherapy, mitochondrial toxins such as cyanide or hydrogen sulphide, or exposure to chemicals such as organophosphates; demyelination due to a dietary deficiency such as vitamin B12 deficiency, vitamin E deficiency and copper deficiency; or demyelination which has unknown causes or multiple causes such as trigeminal neuralgia, Marchiafava-Bignami disease, and Bell's palsy in a subject in need thereof, comprising administering to the subject an effective amount of a compound described herein, e.g., a SHP1 inhibitor described herein or a SHP2 inhibitor described herein, or pharmaceutical composition thereof.

### BRIEF DESCRIPTION OF THE DRAWINGS

FIG. 1A shows exemplary dose-dependent reductions in expression of matrix remodeling genes with Compound A.

FIG. 1B shows exemplary dose-dependent reductions in expression of MMP12 protein with Compound A.

FIG. 2A shows exemplary dose-dependent reductions in expression of cytokine genes with Compound A.

FIG. 2B shows exemplary dose-dependent reductions in expression of Interleukin-33 with Compound A.

FIG. 3A shows an exemplary reduction in expression of cytokine genes with Compound B.

FIG. 3B shows an exemplary reduction in expression of matrix remodeling genes with Compound B.

FIG. 4A shows an exemplary reduction in expression of astrocyte marker genes with Compound B.

FIG. 4B shows an exemplary increase in expression of neuronal marker genes with Compound B.

FIG. 5A shows exemplary results of reductions of expression of cytokine genes with Compound C.

FIG. 5B shows exemplary results of reductions of expression of matrix remodeling genes with Compound C.

FIG. 5C shows an exemplary reduction in expression of astrocyte marker genes with Compound C.

FIG. 5D shows an exemplary increase in expression of neuronal marker genes with Compound C.

### DETAILED DESCRIPTION

The features and other details of the disclosure will now be more particularly described. Certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

Compounds

In some embodiments, compounds of the present disclosure are contemplated as SHP1 inhibitors, e.g., allosteric SHP1 inhibitors.

In some embodiments, compounds of the present disclosure are contemplated as SHP2 inhibitors, e.g., allosteric SHP2 inhibitors.

In some embodiments, the SHP2 inhibitor is a compound disclosed in US20190210977, WO2019/118909, WO2019/075265, WO2019/051084, WO2018/136265, WO2018/136264, WO2018/013597, WO2019/199792, WO2020/076723, or WO2020132597, all of which are incorporated herein by reference in their entireties. In some embodiments, the SHP2 inhibitor is a compound disclosed in PCT/US2017/041577 (WO 2018/013597) or WO2018136265, all of which are incorporated herein by reference in their entireties. In some embodiments, the SHP2 inhibitor is a compound disclosed in US20190231805, U.S. Ser. No. 10/130,629, U.S. Ser. No. 10/301,278, U.S. Ser. No. 10/336,774, WO2010/054045, WO2011/120902, WO2018/130928, WO2015/107495, WO2016/203406, WO2017/216706, WO2016/151501 WO2020/165734, WO2020/165733, or WO2020/165732, all of which are incorporated herein by reference in their entireties. In other embodiments, the SHP2 inhibitor is a compound disclosed in WO2017/211303, WO2018/172984, or WO2020/063760, all of which are incorporated herein by reference in their entireties. In other embodiments, the SHP2 inhibitor is a compound disclosed in WO2018/057884, WO2018/081091, WO2018/218133, WO2019/067843, WO2019/165073, WO2019/183364, or WO2019/183367, all of which are incorporated herein by reference in their entireties. In some embodiments, the SHP2 inhibitor is a compound disclosed in WO2020/073949, and WO2020/073945, all of which are incorporated herein by reference in their entireties. In some embodiments, the SHP2 inhibitor is a compound disclosed in WO2019/167000 or WO2020/022323, all of which are incorporated herein by reference in their entireties. In some embodiments, the SHP2 inhibitor is a compound disclosed in WO2011/105527, which is incorporated herein by reference in its entirety. In some embodiments, the SHP2 inhibitor is a compound disclosed in WO2001/016097, which is incorporated herein by reference in its entirety. In some embodiments, the SHP2 inhibitor is a compound disclosed in WO2012/041524 or WO2006/128909, all of which are incorporated herein by reference in their entireties. In some embodiments, the SHP2 inhibitor is a compound disclosed in WO2016/196591, WO2014/176488, WO2014/055768, or WO2010/118241, all of which are incorporated herein by reference in their entireties. In some embodiments, the SHP2 inhibitor is a compound disclosed in WO2001/019831 or WO1999/046267, all of which are incorporated herein by reference in their entireties. In some embodiments, the SHP2 inhibitor is a compound disclosed in U.S. Pat. No. 8,637,684, which is incorporated herein by reference in its entirety. In some embodiments, the SHP2 inhibitor is a compound disclosed in WO 2019/213318, WO 2017/210134, WO 2017/156397, U.S. Pat. No. 10,851,110, or WO 2020/033828, all of which are incorporated herein by reference in their entireties. In some embodiments, the SHP2 inhibitor is a compound disclosed in WO2019/182960, which is incorporated herein by reference in its entirety. In some embodiments, the SHP2 inhibitor is a compound disclosed in WO2010/011666 or WO2007/117699, which is incorporated herein by reference in its entirety. In some embodiments, the SHP2 inhibitor is a compound disclosed in WO2019/158019, which is incorporated herein by reference in its entirety. In some embodiments, the SHP2 inhibitor is a compound disclosed in WO2019/051469, which is incorporated herein by reference in its entirety. In some embodiments, the SHP2 inhibitor is a compound disclosed in WO2017/100279, which is incorporated herein by reference in its entirety. In some embodiments, the SHP2 inhibitor is a compound disclosed in US2011/0257184, which is incorporated herein by reference in its entirety. In some embodiments, the SHP2 inhibitor is a compound disclosed in WO2004/062664 or WO2004/060878, all of which are incorporated herein by reference in their entireties. In some embodiments, the SHP2 inhibitor is a compound disclosed in WO1996/030332, WO2006/039527, or WO2008/067270, all of which are incorporated herein by reference in their entireties. In some embodiments, the SHP2 inhibitor is a compound disclosed in WO2020/033286, which is incorporated herein by reference in its entirety. In some embodiments, the SHP2 inhibitor is a compound disclosed in WO2002/011722, which is incorporated herein by reference in its entirety. In some embodiments, the SHP2 inhibitor is a compound disclosed in US2004/0043434 or WO2005/094314, all of which are incorporated herein by reference in their entireties. In some embodiments, the SHP2 inhibitor is a compound disclosed in WO2009/135000, which is incorporated herein by reference in its entirety. In some embodiments, the SHP2 inhibitor is a compound disclosed in WO2011/110546, which is incorporated herein by reference in its entirety. In some embodiments, the SHP2 inhibitor is a compound disclosed in WO2019/233810, which is incorporated herein by reference in its entirety. In some embodiments, the SHP2 inhibitor is a compound disclosed in WO2020/177653, which is incorporated herein by reference in its entirety. In some embodiments, the SHP2 inhibitor is a compound disclosed in WO2020/201991 or WO2020/081848, all of which are incorporated herein by reference in their entireties. In some embodiments, the SHP2 inhibitor is a compound disclosed in WO2020/156243 or WO2020/156242, all of which are incorporated herein by reference in their entireties. In some embodiments, the SHP2 inhibitor is a compound disclosed in WO2020/210384 or WO2020/181283, all of which incorporated herein by reference in their entireties. In some embodiments, the SHP2 inhibitor is a compound disclosed in WO2020/061103, which is incorporated herein by reference in its entirety. In some embodiments, the SHP2 inhibitor is a compound disclosed in WO2020/094104, which is incorporated herein by reference in its entirety. In some embodiments, the SHP2 inhibitor is a compound disclosed in WO2020/108590, which is incorporated herein by reference in its entirety. In some embodiments, the SHP2 inhibitor is a compound disclosed in WO2020/118066, which is incorporated herein by reference in its entirety. In some embodiments, the SHP2 inhibitor is a compound disclosed in WO2019/126736, which is incorporated herein by reference in its entirety. In some embodiments, the SHP2 inhibitor is a compound disclosed in U.S. Pat. No. 10,844,079, which is incorporated herein by reference in its entirety.

In some embodiments, the SHP2 inhibitor is:

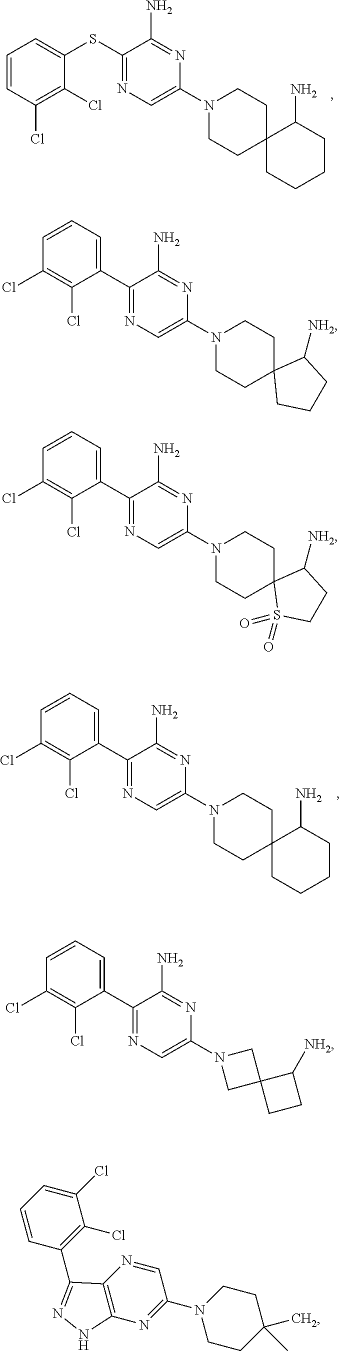

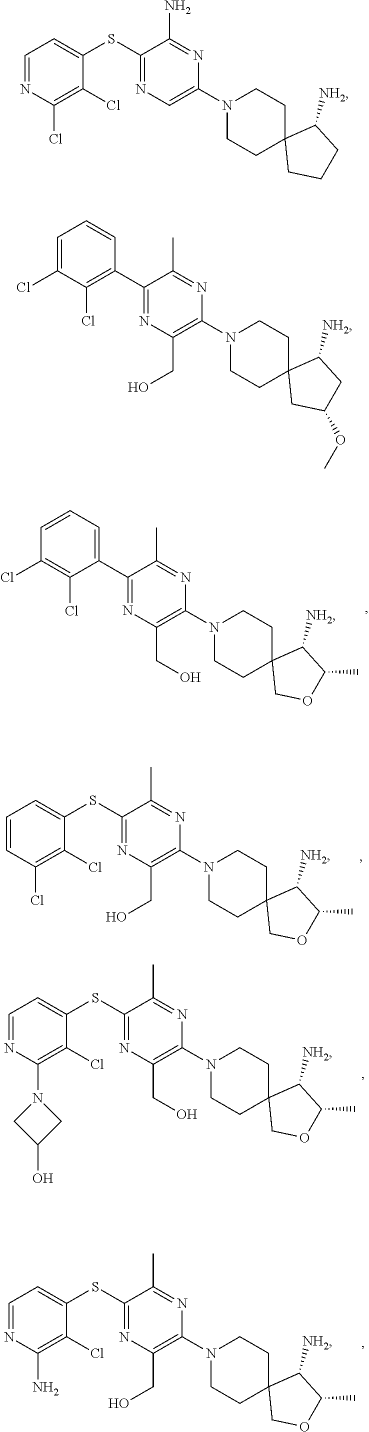 [SMILES: Nc1nc(N2CCC3(CCC[C@H]3N)CC2)cnc1Sc1ccnc(Cl)c1Cl ; CO[C@H]1C[C@@H](N)C2(CCN(c3nc(C)c(-c4cccc(Cl)c4Cl)nc3CO)CC2)C1 ; Cc1nc(N2CCC3(CC2)CO[C@@H](C)[C@H]3N)c(CO)nc1-c1cccc(Cl)c1Cl ; Cc1nc(N2CCC3(CC2)CO[C@@H](C)[C@H]3N)c(CO)nc1Sc1cccc(Cl)c1Cl ; Cc1nc(N2CCC3(CC2)CO[C@@H](C)[C@H]3N)c(CO)nc1Sc1ccnc(N2CC(O)C2)c1Cl ; Cc1nc(N2CCC3(CC2)CO[C@@H](C)[C@H]3N)c(CO)nc1Sc1ccnc(N)c1Cl]

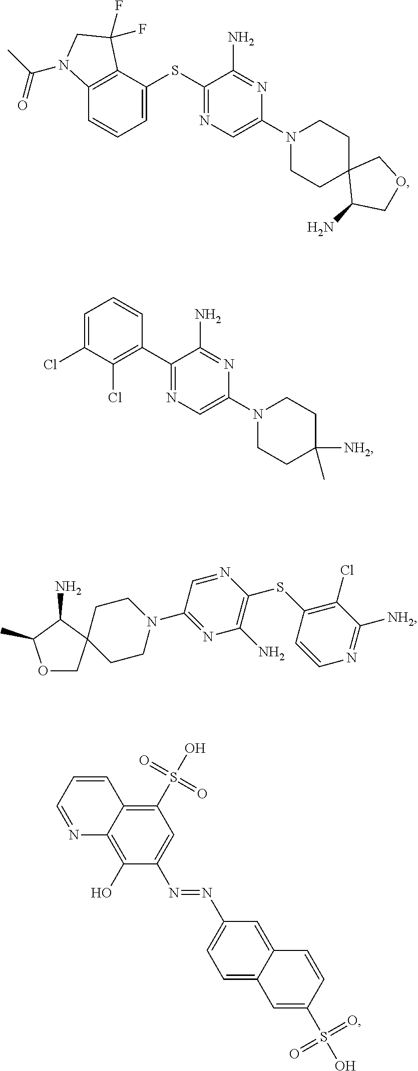 [SMILES: CC(=O)N1CC(F)(F)c2c(Sc3ncc(N4CCC5(CC4)COC[C@H]5N)nc3N)cccc21 ; CC1(N)CCN(c2cnc(-c3cccc(Cl)c3Cl)c(N)n2)CC1 ; C[C@@H]1OCC2(CCN(c3cnc(Sc4ccnc(N)c4Cl)c(N)n3)CC2)[C@@H]1N ; O=S(=O)(O)c1ccc2cc(/N=N/c3cc(S(=O)(=O)O)c4cccnc4c3O)ccc2c1]

or a pharmaceutically acceptable salt thereof.

In some embodiments, the SHP2 inhibitor is:

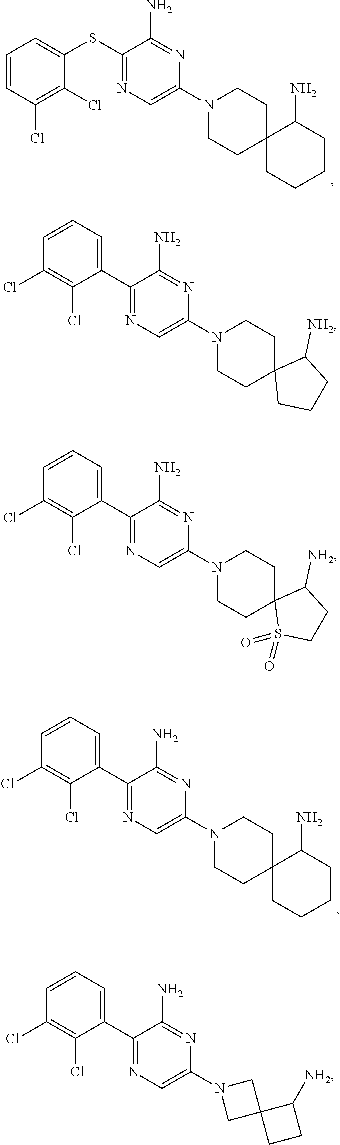 [SMILES: Nc1nc(N2CCC3(CCCCC3N)CC2)cnc1Sc1cccc(Cl)c1Cl ; Nc1nc(N2CCC3(CCCC3N)CC2)cnc1-c1cccc(Cl)c1Cl ; Nc1nc(N2CCC3(CC2)C(N)CCS3(=O)=O)cnc1-c1cccc(Cl)c1Cl ; Nc1nc(N2CCC3(CCCCC3N)CC2)cnc1-c1cccc(Cl)c1Cl ; Nc1nc(N2CC3(CCC3N)C2)cnc1-c1cccc(Cl)c1Cl]

or a pharmaceutically acceptable salt thereof.

In some embodiments, the SHP2 inhibitor is:

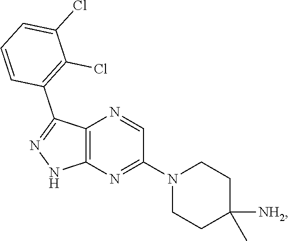 [SMILES: CC1(N)CCN(c2cnc3c(-c4cccc(Cl)c4Cl)n[nH]c3n2)CC1]

or a pharmaceutically acceptable salt thereof. The compound

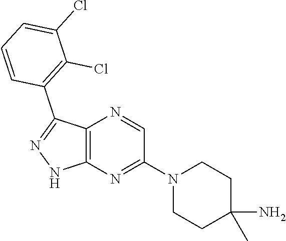 [SMILES: CC1(N)CCN(c2cnc3c(-c4cccc(Cl)c4Cl)n[nH]c3n2)CC1]

is also known as IACS-13909 and referred to herein as “Compound C.”

In some embodiments, the SHP2 inhibitor is:

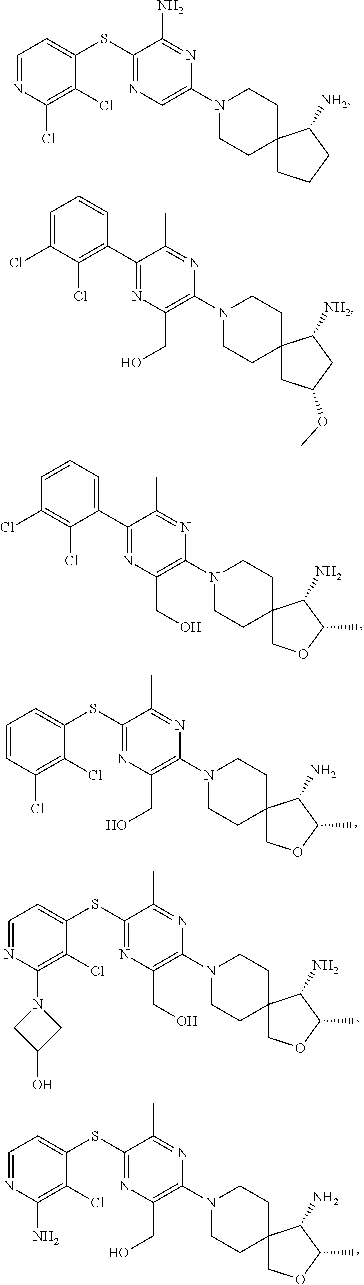 [SMILES: Nc1nc(N2CCC3(CCC[C@H]3N)CC2)cnc1Sc1ccnc(Cl)c1Cl ; CO[C@H]1C[C@@H](N)C2(CCN(c3nc(C)c(-c4cccc(Cl)c4Cl)nc3CO)CC2)C1 ; Cc1nc(N2CCC3(CC2)CO[C@@H](C)[C@H]3N)c(CO)nc1-c1cccc(Cl)c1Cl ; Cc1nc(N2CCC3(CC2)CO[C@@H](C)[C@H]3N)c(CO)nc1Sc1cccc(Cl)c1Cl ; Cc1nc(N2CCC3(CC2)CO[C@@H](C)[C@H]3N)c(CO)nc1Sc1ccnc(N2CC(O)C2)c1Cl ; Cc1nc(N2CCC3(CC2)CO[C@@H](C)[C@H]3N)c(CO)nc1Sc1ccnc(N)c1Cl]

or a pharmaceutically acceptable salt thereof.

In some embodiments, the SHP2 inhibitor is

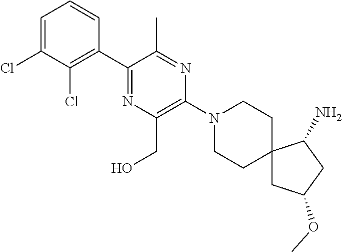 [SMILES: CO[C@H]1C[C@@H](N)C2(CCN(c3nc(C)c(-c4cccc(Cl)c4Cl)nc3CO)CC2)C1]

or a pharmaceutically acceptable salt thereof.

In some embodiments, the SHP2 inhibitor is

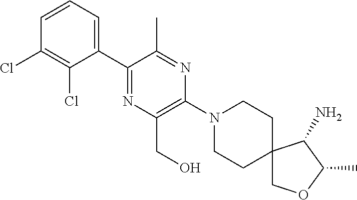 [SMILES: Cc1nc(N2CCC3(CC2)CO[C@@H](C)[C@H]3N)c(CO)nc1-c1cccc(Cl)c1Cl]

or a pharmaceutically acceptable salt thereof. The compound

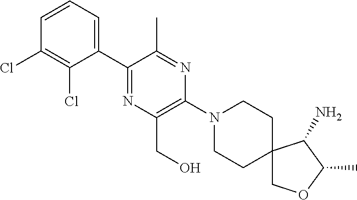 [SMILES: Cc1nc(N2CCC3(CC2)CO[C@@H](C)[C@H]3N)c(CO)nc1-c1cccc(Cl)c1Cl]

is also known as RMC-0694550 or RMC-4550 and is also described herein as Compound A.

In some embodiments, the SHP2 inhibitor is

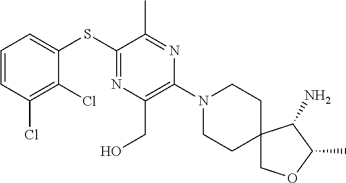 [SMILES: Cc1nc(N2CCC3(CC2)CO[C@@H](C)[C@H]3N)c(CO)nc1Sc1cccc(Cl)c1Cl]

or a pharmaceutically acceptable salt thereof.

In some embodiments, the SHP2 inhibitor is

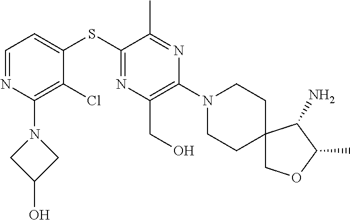 [SMILES: Cc1nc(N2CCC3(CC2)CO[C@@H](C)[C@H]3N)c(CO)nc1Sc1ccnc(N2CC(O)C2)c1Cl]

or a pharmaceutically acceptable salt thereof.

In some embodiments, the SHP2 inhibitor is

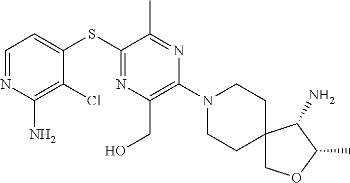 [SMILES: Cc1nc(N2CCC3(CC2)CO[C@@H](C)[C@H]3N)c(CO)nc1Sc1ccnc(N)c1Cl]

or a pharmaceutically acceptable salt thereof.

In some embodiments, SHP2 inhibitor is

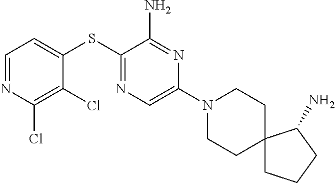 [SMILES: Nc1nc(N2CCC3(CCC[C@H]3N)CC2)cnc1Sc1ccnc(Cl)c1Cl]

or a pharmaceutically acceptable salt thereof. The compound

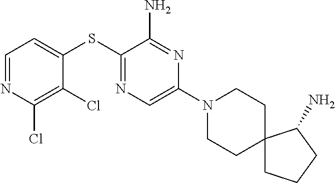 [SMILES: Nc1nc(N2CCC3(CCC[C@H]3N)CC2)cnc1Sc1ccnc(Cl)c1Cl]

is also known as RMC-0693943 or RMC-3943.

In some embodiments, SHP2 inhibitor is

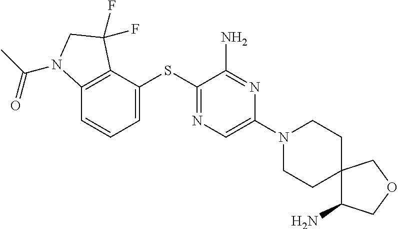 [SMILES: CC(=O)N1CC(F)(F)c2c(Sc3ncc(N4CCC5(CC4)COC[C@H]5N)nc3N)cccc21]

or a pharmaceutically acceptable salt thereof.

In some embodiments, SHP2 inhibitor is

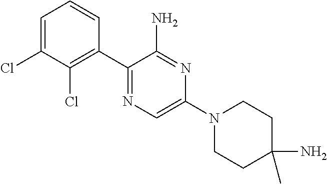 [SMILES: CC1(N)CCN(c2cnc(-c3cccc(Cl)c3Cl)c(N)n2)CC1]

or a pharmaceutically acceptable salt thereof. The compound

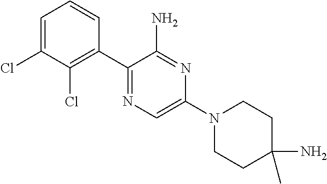 [SMILES: CC1(N)CCN(c2cnc(-c3cccc(Cl)c3Cl)c(N)n2)CC1]

is also known as SHP099.

In some embodiments, SHP2 inhibitor is

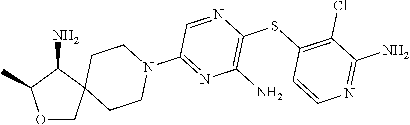 [SMILES: C[C@@H]1OCC2(CCN(c3cnc(Sc4ccnc(N)c4Cl)c(N)n3)CC2)[C@@H]1N]

or a pharmaceutically acceptable salt thereof. The compound

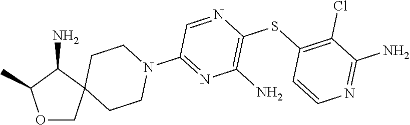 [SMILES: C[C@@H]1OCC2(CCN(c3cnc(Sc4ccnc(N)c4Cl)c(N)n3)CC2)[C@@H]1N]

is also known as TNO155.

In some embodiments, the SHP2 inhibitor is

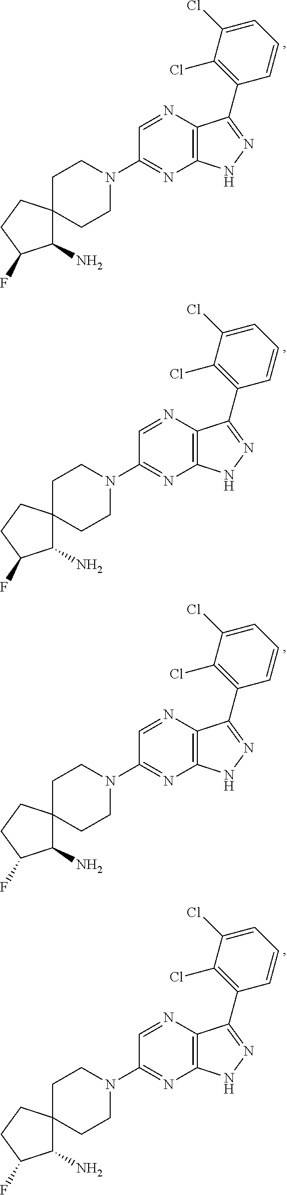 [SMILES: N[C@H]1[C@@H](F)CCC12CCN(c1cnc3c(-c4cccc(Cl)c4Cl)n[nH]c3n1)CC2 ; N[C@@H]1[C@@H](F)CCC12CCN(c1cnc3c(-c4cccc(Cl)c4Cl)n[nH]c3n1)CC2 ; N[C@H]1[C@H](F)CCC12CCN(c1cnc3c(-c4cccc(Cl)c4Cl)n[nH]c3n1)CC2 ; N[C@@H]1[C@H](F)CCC12CCN(c1cnc3c(-c4cccc(Cl)c4Cl)n[nH]c3n1)CC2]

or a pharmaceutically acceptable salt thereof.

In some embodiments, the SHP2 inhibitor is

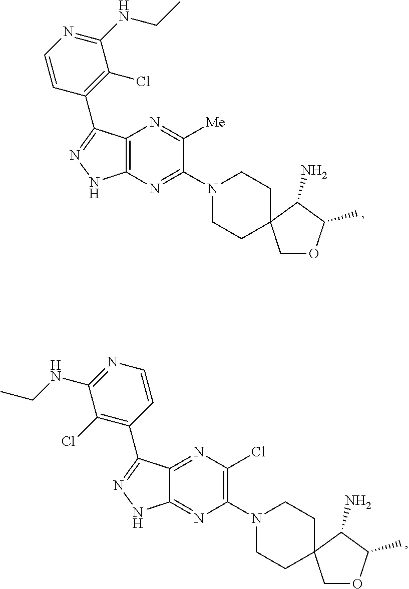

or pharmaceutically acceptable salt thereof.

In one embodiment, a compound described herein can inhibit both SHP1 and SHP2 activity. In some embodiments, the SHP1 inhibitor is a compound disclosed in WO2017/078499, which is incorporated herein by reference in its entirety. In some embodiments, the SHP1 inhibitor is a compound disclosed in U.S. Ser. No. 10/085,976, which is incorporated herein by reference in its entirety. In some embodiments, the SHP1 inhibitor is a compound disclosed in WO2011/105527, which is incorporated herein by reference in its entirety. In some embodiments, the SHP1 inhibitor is a compound disclosed in WO2001/016097, which is incorporated herein by reference in its entirety. In some embodiments, the SHP1 inhibitor is a compound disclosed in WO2012/041524 or WO2006/128909, all of which are incorporated herein by reference in their entireties. In some embodiments, the SHP1 inhibitor is a compound disclosed in WO2003/078959, which is incorporated herein by reference in its entirety. In some embodiments, the SHP1 inhibitor is a compound disclosed in WO2008/002641, which is incorporated herein by reference in its entirety. In some embodiments, the SHP1 inhibitor is a compound disclosed in WO2020/073949 or WO2020/073945, all of which are incorporated herein by reference in their entireties

In some embodiments, the compound that can inhibit both SHP1 and SHP2 activity is

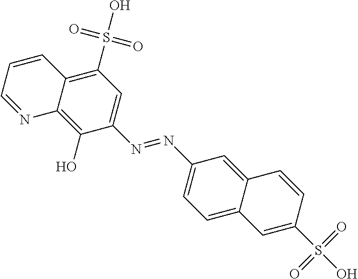 [SMILES: O=S(=O)(O)c1ccc2cc(/N=N/c3cc(S(=O)(=O)O)c4cccnc4c3O)ccc2c1]

or a pharmaceutically acceptable salt thereof. The compound

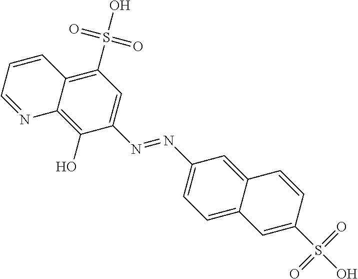 [SMILES: O=S(=O)(O)c1ccc2cc(/N=N/c3cc(S(=O)(=O)O)c4cccnc4c3O)ccc2c1]

is also known as NSC-87877 and is also described herein as Compound B.

### Methods of Treatment

Described herein are methods of treating or preventing disorders, such as inflammation, in subjects in need thereof, comprising administration of a compound described herein.

The present disclosure, in one embodiment, also provides methods of treating or preventing neuroinflammation and related disorders in a subject in need thereof, the method comprising administering to the subject an effective amount of a compound described herein, e.g., a SHP1 inhibitor described herein or a SHP2 inhibitor described herein, or pharmaceutical composition thereof.

Also described herein is a method of treating or preventing neuroinflammation in a subject in need thereof, the method comprising administering to the subject an effective amount of a compound described herein, e.g., a SHP1 inhibitor described herein or a SHP2 inhibitor described herein, or pharmaceutical composition thereof, with the proviso that the SHP2 inhibitor is not: (S)-4-(((S)-1-(12-azanyl)-3-(4-(difluoro(phosphono)methyl)phenyl)-1-oxopropan-2-yl)amino)-3-((S)-3-(4-(difluoro(phosphono)methyl)phenyl)-2-pentadecanamidopropanamido)-4-oxobutanoic acid, ((4-((S)-3-(((S)-1-amino-6-(4-ethylbenzamido)-1-oxohexan-2-yl)amino)-2-((S)-2-(2-(((1R,2R,5S)-2-isopropyl-5-methylcyclohexyl)oxy)acetamido)-3-phenylpropanamido)-3-oxopropyl)phenyl)difluoromethyl)phosphonic acid, ((4-((S)-3-(((S)-1-amino-3-(2-(4-hydroxy-3-methoxyphenyl)acetamido)-1-oxopropan-2-yl)amino)-5-(3-iodobenzamido)-1-oxopentan-2-yl)amino)-6-hydroxy-3-iodo-1-methyl-2-(3-(2-oxo-2-((4-(thiophen-3-yl)phenyl)amino)acetamido)phenyl)-1H-indole-5-carboxylic acid, ((4-((S)-3-(((S)-1-(((S)-1-amino-3-(2-(4-hydroxy-3-methoxyphenyl)acetamido)-1-oxopropan-2-yl)amino)-5-(3-iodobenzamido)-1-oxopentan-2-yl)amino)-2-(3-bromo-4-methylbenzamido)-3-oxopropyl)phenyl)difluoromethyl)difluoromethyl)phosphonic acid, 3-((3-Chlorophenyl)ethynyl)-2-(4-(2-(cyclopropylamino)-2-oxoethoxy)phenyl)-6-hydroxybenzofuran-5-carboxylic acid, or 2-(3-(2-(3-bromo-5-iodobenzamido)acetamido)phenyl)-6-hydroxy-3-iodo-1-methyl-1H-indole-5-caroxylic acid.

Also described herein is a method of treating or preventing a neuroinflammatory disorder in a subject in need thereof, the method comprising administering to the subject an effective amount of a compound described herein, e.g., a SHP1 inhibitor described herein or a SHP2 inhibitor described herein, or pharmaceutical composition thereof.

Also described herein is a method of treating or preventing a disorder associated with neuroinflammation in a subject in need thereof, the method comprising administering to the subject an effective amount of a compound described herein, e.g., a SHP1 inhibitor described herein or a SHP2 inhibitor described herein, or pharmaceutical composition thereof. In some embodiments, the neuroinflammation is associated with a separate disorder in the subject.

Also described herein is a method of treating or preventing Multiple Sclerosis, Parkinson's disease, Multiple System Atrophy, Corticobasal Degeneration, Progressive Supranuclear Paresis, Guillain-Barre Syndrome (GBS), chronic inflammatory demyelinating polyneuropathy (CIDP), viral encephalitis, cerebrovascular accidents, or cranial trauma in a subject in need thereof, comprising administering to the subject an effective amount of a compound described herein, e.g., a SHP1 inhibitor described herein or a SHP2 inhibitor described herein, or pharmaceutical composition thereof. In some embodiments, the Multiple Sclerosis is Relapse Remitting Multiple Sclerosis, Secondary Progressive Multiple Sclerosis or Primary Progressive Multiple Sclerosis.

Additionally described herein is a method of treating or preventing genetic disorders resulting in gain-of-function in SHP2 such as Noonan syndrome and Leopard syndrome, genetic disorders resulting in loss-of-function in the ras signaling pathway including Legius syndrome, neurodegenerative diseases such as Alzheimer's disease, Parkinson's disease, dementia with Lewy bodies, multiple system atrophy, Huntington's disease, spinocerebellar ataxias (e.g., SCA1, SCA2, SCA3, SCA6, SCAT, and SCA17), spinobulbar muscular atrophy (SBMA) or Kennedy disease, dentatorubropallidoluysian atrophy (DRPLA), ALS, AIDS dementia, frontotemporal dementia, corticobasal ganglionic degeneration, progressive supranuclear palsy, Creutzfeldt-Jakob disease, Gerstmann-Straussler-Scheinker syndrome, fatal familial insomnia, corticobasal ganglionic degeneration, hereditary spastic paraplegia, multiple sclerosis, neuromyelitis optica (Devic's disease), concentric sclerosis (Baló's disease), encephalomyelitis including acute disseminated encephalomyelitis (ADEM), acute haemorrhagic leucoencephalitis (AHL), Guillain-Barre Syndrome, chronic inflammatory demyelinating polyneuropathy (CIDP), transverse myelitis, Schilder's disease, fibromyalgia, and optic neuritis; demyelination due to injury such as spinal cord injury, traumatic brain injury, cerebral palsy, neuropathy (e.g. neuropathy due to diabetes, chronic renal failure, hypothyroidism, liver failure, or compression of the nerve (e.g. in Bell's palsy) and post radiation injury; demyelination due to hypoxic-ischaemic events such as stroke, acute ischemic optic neuropathy, or other ischemia, and carbon monoxide exposure; demyelination due to metabolic disruption such as central pontine myelolysis (CPM) or extrapontine myelinolysis (EPM); demyelination due to inherited conditions such as Charcot-Marie-Tooth disease (CMT), Sjogren-Larsson syndrome, Refsum disease, Krabbe disease, Canavan disease, Alexander disease, Friedreich's ataxia, Pelizaeus-Merzbacher disease, Bassen-Kornzweig syndrome, metachromatic leukodystrophy (MLD), adrenoleukodystrophy, Leber's optic neuropathy, and nerve damage due to pernicious anemia; demyelination due to a viral infection such as progressive multifocal leukoencephalopathy (PML), Lyme disease, tabes dorsalis due to untreated syphilis, HIV, or subacute sclerosing panencephalitis due to measles virus; demyelination due to toxic exposure such as chronic alcoholism (which is a possible cause of Marchiafava-Bignami disease), chemotherapy, mitochondrial toxins such as cyanide or hydrogen sulphide, or exposure to chemicals such as organophosphates; demyelination due to a dietary deficiency such as vitamin B12 deficiency, vitamin E deficiency and copper deficiency; or demyelination which has unknown causes or multiple causes such as trigeminal neuralgia, Marchiafava-Bignami disease, and Bell's palsy in a subject in need thereof, comprising administering to the subject an effective amount of a compound described herein, e.g., a SHP1 inhibitor described herein or a SHP2 inhibitor described herein, or pharmaceutical composition thereof.

In some embodiments, the compound is administered orally, parenterally, rectally, transdermally, intradermally, intrathecally, subcutaneously, intravenously, intramuscularly, or intranasally. In some embodiments, the compound is orally administered.

The present disclosure, in one embodiment, also provides methods of treating neuroinflammation and related disorders in a subject in need thereof, the method comprising administering to the subject a therapeutically effective amount of a compound described herein, e.g., a SHP1 inhibitor described herein or a SHP2 inhibitor described herein, or pharmaceutical composition thereof.

Also described herein is a method of treating neuroinflammation in a subject in need thereof, the method comprising administering to the subject a therapeutically effective amount of a compound described herein, e.g., a SHP1 inhibitor described herein or a SHP2 inhibitor described herein, or pharmaceutical composition thereof.

Also described herein is a method of treating a neuroinflammatory disorder in a subject in need thereof, the method comprising administering to the subject a therapeutically effective amount of a compound described herein, e.g., a SHP1 inhibitor described herein or a SHP2 inhibitor described herein, or pharmaceutical composition thereof.

Also described herein is a method of treating a disorder associated with neuroinflammation in a subject in need thereof, the method comprising administering to the subject an effective amount of a compound described herein, e.g., a SHP1 inhibitor described herein or a SHP2 inhibitor described herein, or pharmaceutical composition thereof. In some embodiments, the neuroinflammation is associated with a separate disorder in the subject.

Also described herein is a method of treating or preventing Multiple Sclerosis, Parkinson's disease, Multiple System Atrophy, Corticobasal Degeneration, Progressive Supranuclear Paresis, Guillain-Barre Syndrome (GBS), chronic inflammatory demyelinating polyneuropathy (CIDP), viral encephalitis, cerebrovascular accidents, or cranial trauma in a subject in need thereof, comprising administering to the subject a therapeutically effective amount of a compound described herein, e.g., a SHP1 inhibitor described herein or a SHP2 inhibitor described herein, or pharmaceutical composition thereof. In some embodiments, the Multiple Sclerosis is Relapse Remitting Multiple Sclerosis, Secondary Progressive Multiple Sclerosis or Primary Progressive Multiple Sclerosis.

Additionally described herein is a method of treating genetic disorders such as Noonan syndrome and Leopard syndrome, genetic disorders resulting in loss-of-function in the ras signaling pathway including Legius syndrome, neurodegenerative diseases such as Alzheimer's disease, Parkinson's disease, dementia with Lewy bodies, multiple system atrophy, Huntington's disease, spinocerebellar ataxias (e.g., SCA1, SCA2, SCA3, SCA6, SCAT, and SCA17), spinobulbar muscular atrophy (SBMA) or Kennedy disease, dentatorubropallidoluysian atrophy (DRPLA), ALS, AIDS dementia, frontotemporal dementia, corticobasal ganglionic degeneration, progressive supranuclear palsy, Creutzfeldt-Jakob disease, Gerstmann-Straussler-Scheinker syndrome, fatal familial insomnia, corticobasal ganglionic degeneration, hereditary spastic paraplegia, multiple sclerosis, neuromyelitis optica (Devic's disease), concentric sclerosis (Baló's disease), encephalomyelitis including acute disseminated encephalomyelitis (ADEM), acute haemorrhagic leucoencephalitis (AHL), Guillain-Barre Syndrome, chronic inflammatory demyelinating polyneuropathy (CIDP), transverse myelitis, Schilder's disease, fibromyalgia, and optic neuritis; demyelination due to injury such as spinal cord injury, traumatic brain injury, cerebral palsy, neuropathy (e.g. neuropathy due to diabetes, chronic renal failure, hypothyroidism, liver failure, or compression of the nerve (e.g. in Bell's palsy) and post radiation injury; demyelination due to hypoxic-ischaemic events such as stroke, acute ischemic optic neuropathy, or other ischemia, and carbon monoxide exposure; demyelination due to metabolic disruption such as central pontine myelolysis (CPM) or extrapontine myelinolysis (EPM); demyelination due to inherited conditions such as Charcot-Marie-Tooth disease (CMT), Sjogren-Larsson syndrome, Refsum disease, Krabbe disease, Canavan disease, Alexander disease, Friedreich's ataxia, Pelizaeus-Merzbacher disease, Bassen-Kornzweig syndrome, metachromatic leukodystrophy (MLD), adrenoleukodystrophy, Leber's optic neuropathy, and nerve damage due to pernicious anemia; demyelination due to a viral infection such as progressive multifocal leukoencephalopathy (PML), Lyme disease, tabes dorsalis due to untreated syphilis, HIV, or subacute sclerosing panencephalitis due to measles virus; demyelination due to toxic exposure such as chronic alcoholism (which is a possible cause of Marchiafava-Bignami disease), chemotherapy, mitochondrial toxins such as cyanide or hydrogen sulphide, or exposure to chemicals such as organophosphates; demyelination due to a dietary deficiency such as vitamin B12 deficiency, vitamin E deficiency and copper deficiency; or demyelination which has unknown causes or multiple causes such as trigeminal neuralgia, Marchiafava-Bignami disease, and Bell's palsy in a subject in need thereof, comprising administering to the subject a therapeutically effective amount of a compound described herein, e.g., a SHP1 inhibitor described herein or a SHP2 inhibitor described herein, or pharmaceutical composition thereof.

The present disclosure also provides methods of treating or preventing neuroflammation and other disorders in a subject in need thereof, comprising administering to the subject an effective amount of a SHP2 inhibitor, e.g., a SHP2 inhibitor described herein. In one embodiment, the methods provided herein can be used to treat or prevent disorders associated with mutations in the PTPN11 gene.

In another embodiment, the present disclosure provides methods of treating or preventing neuroinflammation and related disorders in a subject in need thereof, the method comprising administering to the subject an effective amount of a SHP2 inhibitor, e.g., an allosteric SHP2 inhibitor, or a pharmaceutical composition thereof.

In an embodiment, the methods described herein provide a method of treating or preventing neuroinflammation in a subject in need thereof, the method comprising administering to the subject an effective amount of a SHP2 inhibitor, e.g., a SHP2 inhibitor described herein.

In an embodiment, the methods described herein provide a method of treating or preventing a neuroinflammatory disorder in a subject in need thereof, the method comprising administering to the subject an effective amount of a SHP2 inhibitor, e.g., a SHP2 inhibitor described herein.

In an embodiment, the methods described herein provide a method of treating or preventing a disorder associated with neuroinflammation in a subject in need thereof, the method comprising administering to the subject an effective amount of a SHP2 inhibitor, e.g., a SHP2 inhibitor described herein. In some embodiments, the neuroinflammation is associated with a separate disorder in the subject.

In an embodiment, the methods described herein provide a method of treating or preventing Multiple Sclerosis, Parkinson's disease, Multiple System Atrophy, Corticobasal Degeneration, Progressive Supranuclear Paresis, Guillain-Barre Syndrome (GBS), chronic inflammatory demyelinating polyneuropathy (CIDP), viral encephalitis, cerebrovascular accidents, or cranial trauma in a subject in need thereof, comprising administering to the subject an effective amount of a SHP2 inhibitor, e.g., a SHP2 inhibitor described herein. In some embodiments, the Multiple Sclerosis is Relapse Remitting Multiple Sclerosis, Secondary Progressive Multiple Sclerosis or Primary Progressive Multiple Sclerosis.

Additionally described herein are other methods including a method of treating or preventing genetic disorders such as Noonan syndrome and Leopard syndrome, genetic disorders resulting in loss-of-function in the ras signaling pathway including Legius syndrome, neurodegenerative diseases such as Alzheimer's disease, Parkinson's disease, dementia with Lewy bodies, multiple system atrophy, Huntington's disease, spinocerebellar ataxias (e.g., SCA1, SCA2, SCA3, SCA6, SCAT, and SCA17), spinobulbar muscular atrophy (SBMA) or Kennedy disease, dentatorubropallidoluysian atrophy (DRPLA), ALS, AIDS dementia, frontotemporal dementia, corticobasal ganglionic degeneration, progressive supranuclear palsy, Creutzfeldt-Jakob disease, Gerstmann-Straussler-Scheinker syndrome, fatal familial insomnia, corticobasal ganglionic degeneration, hereditary spastic paraplegia, multiple sclerosis, neuromyelitis optica (Devic's disease), concentric sclerosis (Baló's disease), encephalomyelitis including acute disseminated encephalomyelitis (ADEM), acute haemorrhagic leucoencephalitis (AHL), Guillain-Barre Syndrome, chronic inflammatory demyelinating polyneuropathy (CIDP), transverse myelitis, Schilder's disease, fibromyalgia, and optic neuritis; demyelination due to injury such as spinal cord injury, traumatic brain injury, cerebral palsy, neuropathy (e.g. neuropathy due to diabetes, chronic renal failure, hypothyroidism, liver failure, or compression of the nerve (e.g. in Bell's palsy) and post radiation injury; demyelination due to hypoxic-ischaemic events such as stroke, acute ischemic optic neuropathy, or other ischemia, and carbon monoxide exposure; demyelination due to metabolic disruption such as central pontine myelolysis (CPM) or extrapontine myelinolysis (EPM); demyelination due to inherited conditions such as Charcot-Marie-Tooth disease (CMT), Sjogren-Larsson syndrome, Refsum disease, Krabbe disease, Canavan disease, Alexander disease, Friedreich's ataxia, Pelizaeus-Merzbacher disease, Bassen-Kornzweig syndrome, metachromatic leukodystrophy (MLD), adrenoleukodystrophy, Leber's optic neuropathy, and nerve damage due to pernicious anemia; demyelination due to a viral infection such as progressive multifocal leukoencephalopathy (PML), Lyme disease, tabes dorsalis due to untreated syphilis, HIV, or subacute sclerosing panencephalitis due to measles virus; demyelination due to toxic exposure such as chronic alcoholism (which is a possible cause of Marchiafava-Bignami disease), chemotherapy, mitochondrial toxins such as cyanide or hydrogen sulphide, or exposure to chemicals such as organophosphates; demyelination due to a dietary deficiency such as vitamin B12 deficiency, vitamin E deficiency and copper deficiency; or demyelination which has unknown causes or multiple causes such as trigeminal neuralgia, Marchiafava-Bignami disease, and Bell's palsy in a subject in need thereof, comprising administering to the subject an effective amount of a SHP2 inhibitor, e.g., a SHP2 inhibitor described herein. In some embodiments, the genetic disorders such as Noonan syndrome and Leopard syndrome result from gain-of-function in SHP2.

In an embodiment, described herein is a method of treating neuroinflammation in a subject in need thereof, the method comprising administering to the subject a therapeutically effective amount of a SHP2 inhibitor, e.g., a SHP2 inhibitor described herein.

In an embodiment, described herein is a method of treating a neuroinflammatory disorder in a subject in need thereof, the method comprising administering to the subject a therapeutically effective amount of a SHP2 inhibitor, e.g., a SHP2 inhibitor described herein.

In an embodiment, described herein is a method of treating a disorder associated with neuroinflammation in a subject in need thereof, the method comprising administering to the subject a therapeutically effective amount of a SHP2 inhibitor, e.g., a SHP2 inhibitor described herein. In some embodiments, the neuroinflammation is associated with a separate disorder in the subject.

In an embodiment, the methods described herein provide a method of treating Multiple Sclerosis, Parkinson's disease, Multiple System Atrophy, Corticobasal Degeneration, Progressive Supranuclear Paresis, Guillain-Barre Syndrome (GBS), chronic inflammatory demyelinating polyneuropathy (CIDP), viral encephalitis, cerebrovascular accidents, or cranial trauma in a subject in need thereof, comprising administering to the subject a therapeutically effective amount of a SHP2 inhibitor, e.g., a SHP2 inhibitor described herein. In some embodiments, the Multiple Sclerosis is Relapse Remitting Multiple Sclerosis, Secondary Progressive Multiple Sclerosis or Primary Progressive Multiple Sclerosis.

Additionally described herein are other methods including a method of treating genetic disorders such as Noonan syndrome and Leopard syndrome, genetic disorders resulting in loss-of-function in the ras signaling pathway including Legius syndrome, neurodegenerative diseases such as Alzheimer's disease, Parkinson's disease, dementia with Lewy bodies, multiple system atrophy, Huntington's disease, spinocerebellar ataxias (e.g., SCA1, SCA2, SCA3, SCA6, SCAT, and SCA17), spinobulbar muscular atrophy (SBMA) or Kennedy disease, dentatorubropallidoluysian atrophy (DRPLA), ALS, AIDS dementia, frontotemporal dementia, corticobasal ganglionic degeneration, progressive supranuclear palsy, Creutzfeldt-Jakob disease, Gerstmann-Straussler-Scheinker syndrome, fatal familial insomnia, corticobasal ganglionic degeneration, hereditary spastic paraplegia, multiple sclerosis, neuromyelitis optica (Devic's disease), concentric sclerosis (Baló's disease), encephalomyelitis including acute disseminated encephalomyelitis (ADEM), acute haemorrhagic leucoencephalitis (AHL), Guillain-Barre Syndrome, chronic inflammatory demyelinating polyneuropathy (CIDP), transverse myelitis, Schilder's disease, fibromyalgia, and optic neuritis; demyelination due to injury such as spinal cord injury, traumatic brain injury, cerebral palsy, neuropathy (e.g. neuropathy due to diabetes, chronic renal failure, hypothyroidism, liver failure, or compression of the nerve (e.g. in Bell's palsy) and post radiation injury; demyelination due to hypoxic-ischaemic events such as stroke, acute ischemic optic neuropathy, or other ischemia, and carbon monoxide exposure; demyelination due to metabolic disruption such as central pontine myelolysis (CPM) or extrapontine myelinolysis (EPM); demyelination due to inherited conditions such as Charcot-Marie-Tooth disease (CMT), Sjogren-Larsson syndrome, Refsum disease, Krabbe disease, Canavan disease, Alexander disease, Friedreich's ataxia, Pelizaeus-Merzbacher disease, Bassen-Kornzweig syndrome, metachromatic leukodystrophy (MLD), adrenoleukodystrophy, Leber's optic neuropathy, and nerve damage due to pernicious anemia; demyelination due to a viral infection such as progressive multifocal leukoencephalopathy (PML), Lyme disease, tabes dorsalis due to untreated syphilis, HIV, or subacute sclerosing panencephalitis due to measles virus; demyelination due to toxic exposure such as chronic alcoholism (which is a possible cause of Marchiafava-Bignami disease), chemotherapy, mitochondrial toxins such as cyanide or hydrogen sulphide, or exposure to chemicals such as organophosphates; demyelination due to a dietary deficiency such as vitamin B12 deficiency, vitamin E deficiency and copper deficiency; or demyelination which has unknown causes or multiple causes such as trigeminal neuralgia, Marchiafava-Bignami disease, and Bell's palsy in a subject in need thereof, comprising administering to the subject a therapeutically effective amount of a SHP2 inhibitor, e.g., a SHP2 inhibitor described herein. In some embodiments, the genetic disorders such as Noonan syndrome and Leopard syndrome result from gain-of-function in SHP2.

In some embodiments, the SHP2 inhibitor is administered orally, parenterally, rectally, transdermally, intradermally, intrathecally, subcutaneously, intravenously, intramuscularly, or intranasally. In some embodiments, the SHP2 inhibitor is orally administered.

In one embodiment, described herein is a method of treating or preventing neuroinflammation in a subject in need thereof, the method comprising administering to the subject an effective amount of a SHP2 inhibitor described herein, e.g., an allosteric SHP2 inhibitor, or a pharmaceutically acceptable salt thereof, with the proviso that the SHP2 inhibitor is not: (S)-4-(((S)-1-(12-azanyl)-3-(4-(difluoro(phosphono)methyl)phenyl)-1-oxopropan-2-yl)amino)-3-((S)-3-(4-(difluoro(phosphono)methyl)phenyl)-2-pentadecanamidopropanamido)-4-oxobutanoic acid, ((4-((S)-3-(((S)-1-amino-6-(4-ethylbenzamido)-1-oxohexan-2-yl)amino)-2-((S)-2-(2-(((1R,2R,5S)-2-isopropyl-5-methylcyclohexyl)oxy)acetamido)-3-phenylpropanamido)-3-oxopropyl)phenyl)difluoromethyl)phosphonic acid, ((4-((S)-3-(((S)-1-amino-3-(2-(4-hydroxy-3-methoxyphenyl)acetamido)-1-oxopropan-2-yl)amino)-5-(3-iodobenzamido)-1-oxopentan-2-yl)amino)-6-hydroxy-3-iodo-1-methyl-2-(3-(2-oxo-2-((4-(thiophen-3-yl)phenyl)amino)acetamido)phenyl)-1H-indole-5-carboxylic acid, ((4-((S)-3-(((S)-1-(((S)-1-amino-3-(2-(4-hydroxy-3-methoxyphenyl)acetamido)-1-oxopropan-2-yl)amino)-5-(3-iodobenzamido)-1-oxopentan-2-yl)amino)-2-(3-bromo-4-methylbenzamido)-3-oxopropyl)phenyl)difluoromethyl)difluoromethyl)phosphonic acid, 3-((3-Chlorophenyl)ethynyl)-2-(4-(2-(cyclopropylamino)-2-oxoethoxy)phenyl)-6-hydroxybenzofuran-5-carboxylic acid, or 2-(3-(2-(3-bromo-5-iodobenzamido)acetamido)phenyl)-6-hydroxy-3-iodo-1-methyl-1H-indole-5-caroxylic acid.

In one embodiment, described herein is a method of treating or preventing a neuroinflammatory disorder in a subject in need thereof, the method comprising administering to the subject an effective amount of a SHP2 inhibitor described herein, e.g., an allosteric SHP2 inhibitor, or a pharmaceutically acceptable salt thereof, with the proviso that the SHP2 inhibitor is not: (S)-4-(((S)-1-(12-azanyl)-3-(4-(difluoro(phosphono)methyl)phenyl)-1-oxopropan-2-yl)amino)-3-((S)-3-(4-(difluoro(phosphono)methyl)phenyl)-2-pentadecanamidopropanamido)-4-oxobutanoic acid, ((4-((S)-3-(((S)-1-amino-6-(4-ethylbenzamido)-1-oxohexan-2-yl)amino)-2-((S)-2-(2-(((1R,2R,5S)-2-isopropyl-5-methylcyclohexyl)oxy)acetamido)-3-phenylpropanamido)-3-oxopropyl)phenyl)difluoromethyl)phosphonic acid, ((4-((S)-3-(((S)-1-amino-3-(2-(4-hydroxy-3-methoxyphenyl)acetamido)-1-oxopropan-2-yl)amino)-5-(3-iodobenzamido)-1-oxopentan-2-yl)amino)-6-hydroxy-3-iodo-1-methyl-2-(3-(2-oxo-2-((4-(thiophen-3-yl)phenyl)amino)acetamido)phenyl)-1H-indole-5-carboxylic acid, ((4-((S)-3-(((S)-1-(((S)-1-amino-3-(2-(4-hydroxy-3-methoxyphenyl)acetamido)-1-oxopropan-2-yl)amino)-5-(3-iodobenzamido)-1-oxopentan-2-yl)amino)-2-(3-bromo-4-methylbenzamido)-3-oxopropyl)phenyl)difluoromethyl)difluoromethyl)phosphonic acid, 3-((3-Chlorophenyl)ethynyl)-2-(4-(2-(cyclopropylamino)-2-oxoethoxy)phenyl)-6-hydroxybenzofuran-5-carboxylic acid, or 2-(3-(2-(3-bromo-5-iodobenzamido)acetamido)phenyl)-6-hydroxy-3-iodo-1-methyl-1H-indole-5-caroxylic acid.

In one embodiment, described herein is a method of treating or preventing a disorder associated with neuroinflammation in a subject in need thereof, the method comprising administering to the subject an effective amount of a SHP2 inhibitor described herein, e.g., an allosteric SHP2 inhibitor, or a pharmaceutically acceptable salt thereof, with the proviso that the SHP2 inhibitor is not: (S)-4-(((S)-1-(12-azanyl)-3-(4-(difluoro(phosphono)methyl)phenyl)-1-oxopropan-2-yl)amino)-3-((S)-3-(4-(difluoro(phosphono)methyl)phenyl)-2-pentadecanamidopropanamido)-4-oxobutanoic acid, ((4-((S)-3-(((S)-1-amino-6-(4-ethylbenzamido)-1-oxohexan-2-yl)amino)-2-((S)-2-(2-(((1R,2R,5S)-2-isopropyl-5-methylcyclohexyl)oxy)acetamido)-3-phenylpropanamido)-3-oxopropyl)phenyl)difluoromethyl)phosphonic acid, ((4-((S)-3-(((S)-1-amino-3-(2-(4-hydroxy-3-methoxyphenyl)acetamido)-1-oxopropan-2-yl)amino)-5-(3-iodobenzamido)-1-oxopentan-2-yl)amino)-6-hydroxy-3-iodo-1-methyl-2-(3-(2-oxo-2-((4-(thiophen-3-yl)phenyl)amino)acetamido)phenyl)-1H-indole-5-carboxylic acid, ((4-((S)-3-(((S)-1-(((S)-1-amino-3-(2-(4-hydroxy-3-methoxyphenyl)acetamido)-1-oxopropan-2-yl)amino)-5-(3-iodobenzamido)-1-oxopentan-2-yl)amino)-2-(3-bromo-4-methylbenzamido)-3-oxopropyl)phenyl)difluoromethyl)difluoromethyl)phosphonic acid, 3-((3-Chlorophenyl)ethynyl)-2-(4-(2-(cyclopropylamino)-2-oxoethoxy)phenyl)-6-hydroxybenzofuran-5-carboxylic acid, or 2-(3-(2-(3-bromo-5-iodobenzamido)acetamido)phenyl)-6-hydroxy-3-iodo-1-methyl-1H-indole-5-caroxylic acid.

In some embodiments, the neuroinflammation is associated with a separate disorder in the subject.

In one embodiment, described herein is a method of treating or preventing a neurodegenerative disorder in a subject in need thereof, comprising administering to the subject an effective amount of a SHP2 inhibitor described herein, e.g., an allosteric SHP2 inhibitor, or a pharmaceutically acceptable salt thereof, with the proviso that the SHP2 inhibitor is not: (S)-4-(((S)-1-(12-azanyl)-3-(4-(difluoro(phosphono)methyl)phenyl)-1-oxopropan-2-yl)amino)-3-((S)-3-(4-(difluoro(phosphono)methyl)phenyl)-2-pentadecanamidopropanamido)-4-oxobutanoic acid, ((4-((S)-3-(((S)-1-amino-6-(4-ethylbenzamido)-1-oxohexan-2-yl)amino)-2-((S)-2-(2-(((1R,2R,5S)-2-isopropyl-5-methylcyclohexyl)oxy)acetamido)-3-phenylpropanamido)-3-oxopropyl)phenyl)difluoromethyl)phosphonic acid, ((4-((S)-3-(((S)-1-amino-3-(2-(4-hydroxy-3-methoxyphenyl)acetamido)-1-oxopropan-2-yl)amino)-5-(3-iodobenzamido)-1-oxopentan-2-yl)amino)-6-hydroxy-3-iodo-1-methyl-2-(3-(2-oxo-2-((4-(thiophen-3-yl)phenyl)amino)acetamido)phenyl)-1H-indole-5-carboxylic acid, ((4-((S)-3-(((S)-1-(((S)-1-amino-3-(2-(4-hydroxy-3-methoxyphenyl)acetamido)-1-oxopropan-2-yl)amino)-5-(3-iodobenzamido)-1-oxopentan-2-yl)amino)-2-(3-bromo-4-methylbenzamido)-3-oxopropyl)phenyl)difluoromethyl)difluoromethyl)phosphonic acid, 3-((3-Chlorophenyl)ethynyl)-2-(4-(2-(cyclopropylamino)-2-oxoethoxy)phenyl)-6-hydroxybenzofuran-5-carboxylic acid, or 2-(3-(2-(3-bromo-5-iodobenzamido)acetamido)phenyl)-6-hydroxy-3-iodo-1-methyl-1H-indole-5-caroxylic acid.

In some embodiments, the neurodegenerative disorder is Alzheimer's disease, Parkinson's disease, dementia with Lewy bodies, multiple system atrophy, Huntington's disease, spinocerebellar ataxias, spinobulbar muscular atrophy (SBMA) or Kennedy disease, dentatorubropallidoluysian atrophy (DRPLA), ALS, AIDS dementia, frontotemporal dementia, corticobasal ganglionic degeneration, progressive supranuclear palsy, Creutzfeldt-Jakob disease, Gerstmann-Straussler-Scheinker syndrome, fatal familial insomnia, corticobasal ganglionic degeneration, hereditary spastic paraplegia, multiple sclerosis, neuromyelitis optica (Devic's disease), concentric sclerosis (Baló's disease), encephalomyelitis including acute disseminated encephalomyelitis (ADEM), acute haemorrhagic leucoencephalitis (AHL), Guillain-Barre Syndrome, chronic inflammatory demyelinating polyneuropathy (CIDP), transverse myelitis, Schilder's disease, fibromyalgia, or optic neuritis.

In one embodiment, described herein is a method of treating or preventing Multiple Sclerosis, Parkinson's disease, Multiple System Atrophy, Corticobasal Degeneration, Progressive Supranuclear Paresis, Guillain-Barre Syndrome (GBS), chronic inflammatory demyelinating polyneuropathy (CIDP), viral encephalitis, cerebrovascular accidents, or cranial trauma in a subject in need thereof, comprising administering to the subject an effective amount of a SHP2 inhibitor described herein, e.g., an allosteric SHP2 inhibitor, or a pharmaceutically acceptable salt thereof.

In some embodiments, the Multiple Sclerosis is Relapse Remitting Multiple Sclerosis, Secondary Progressive Multiple Sclerosis or Primary Progressive Multiple Sclerosis.

In one embodiment, described herein method of treating or preventing a genetic disorder resulting in gain-of-function in SHP2 in a subject in need thereof, comprising administering to the subject an effective amount of a SHP2 inhibitor described herein, e.g., an allosteric SHP2 inhibitor, or a pharmaceutically acceptable salt thereof.

In some embodiments, the genetic disorder resulting in gain-of-function in SHP2 is Noonan syndrome or Leopard syndrome.

In one embodiment, described herein is a method of treating or preventing a genetic disorder resulting in loss-of-function in the ras signaling pathway in a subject in need thereof, comprising administering to the subject an effective amount of a SHP2 inhibitor described herein, e.g., an allosteric SHP2 inhibitor, or a pharmaceutically acceptable salt thereof.

In some embodiments, the genetic disorder resulting in loss-of-function in the ras signaling pathway is Legius syndrome.

In an embodiment, described herein is a method of treating or preventing demyelination in a subject in need thereof, comprising administering to the subject an effective amount of a SHP2 inhibitor described herein, e.g., an allosteric SHP2 inhibitor, or a pharmaceutically acceptable salt thereof.

In some embodiments, the demyelation is caused by an injury, a hypoxic-ischaemic event, a metabolic disruption, an inherited condition, or an exposure to a toxic substance. In some embodiments, the injury is a spinal cord injury, traumatic brain injury, cerebral palsy, or neuropathy. In some embodiments, the hypoxic-ischaemic event is stroke, acute ischemic optic neuropathy, or other ischemia, or carbon monoxide exposure. In some embodiments, the metabolic disruption is central pontine myelolysis (CPM), or extrapontine myelinolysis (EPM). In some embodiments, the inherited condition is Charcot-Marie-Tooth disease (CMT), Sjogren-Larsson syndrome, Refsum disease, Krabbe disease, Canavan disease, Alexander disease, Friedreich's ataxia, Pelizaeus-Merzbacher disease, Bassen-Kornzweig syndrome, metachromatic leukodystrophy (MLD), adrenoleukodystrophy, Leber's optic neuropathy, or nerve damage due to pernicious anemia. In some embodiments, the exposure to a toxic substance is chronic alcoholism. In an embodiment, described herein is a method of treating or preventing Noonan syndrome, Leopard syndrome, Legius syndrome, Alzheimer's disease, Parkinson's disease, dementia with Lewy bodies, multiple system atrophy, Huntington's disease, spinocerebellar ataxias (e.g., SCA1, SCA2, SCA3, SCA6, SCAT, and SCA17), spinobulbar muscular atrophy (SBMA), Kennedy disease, dentatorubropallidoluysian atrophy (DRPLA), ALS, AIDS dementia, frontotemporal dementia, corticobasal ganglionic degeneration, progressive supranuclear palsy, Creutzfeldt-Jakob disease, Gerstmann-Straussler-Scheinker syndrome, fatal familial insomnia, corticobasal ganglionic degeneration, hereditary spastic paraplegia, multiple sclerosis, neuromyelitis optica (Devic's disease), concentric sclerosis (Baló's disease), acute disseminated encephalomyelitis (ADEM), acute haemorrhagic leucoencephalitis (AHL), Guillain-Barre Syndrome, chronic inflammatory demyelinating polyneuropathy (CIDP), transverse myelitis, Schilder's disease, fibromyalgia, optic neuritis, spinal cord injury, traumatic brain injury, cerebral palsy, neuropathy; stroke, acute ischemic optic neuropathy, or other ischemia, and carbon monoxide exposure; central pontine myelolysis (CPM), extrapontine myelinolysis (EPM), Charcot-Marie-Tooth disease (CMT), Sjogren-Larsson syndrome, Refsum disease, Krabbe disease, Canavan disease, Alexander disease, Friedreich's ataxia, Pelizaeus-Merzbacher disease, Bassen-Kornzweig syndrome, metachromatic leukodystrophy (MLD), adrenoleukodystrophy, Leber's optic neuropathy, nerve damage due to pernicious anemia, progressive multifocal leukoencephalopathy (PML), Lyme disease, tabes dorsalis due to untreated syphilis, HIV, subacute sclerosing panencephalitis due to measles virus, Marchiafava-Bignami disease, chemotherapy, a disorder resulting from exposure to mitochondrial toxins, or exposure to chemicals, vitamin B12 deficiency, vitamin E deficiency, copper deficiency, trigeminal neuralgia, Marchiafava-Bignami disease, or Bell's palsy in a subject in need thereof, comprising administering to the subject an effective amount of a SHP2 inhibitor, e.g., an allosteric SHP2 inhibitor, described herein or a pharmaceutically acceptable salt thereof.

In some embodiments, the neuropathy is neuropathy due to diabetes, chronic renal failure, hypothyroidism, liver failure, or compression of the nerve (e.g. in Bell's palsy), or post radiation injury.

In some embodiments, the SHP2 inhibitor is administered orally, parenterally, rectally, transdermally, intradermally, intrathecally, subcutaneously, intravenously, intramuscularly, or intranasally. In some embodiments, the SHP1 inhibitor or SHP2 inhibitor is orally administered.

Also provided are methods of treating or preventing disorders in a subject in need thereof, comprising administering to the subject a SHP1 inhibitor, e.g., an allosteric SHP1 inhibitor or pharmaceutical composition thereof. In one embodiment, the methods provided herein can be used to treat or prevent disorders associated with mutations in the PTPN6 gene.

In another embodiment, the present disclosure provides methods of treating or preventing neuroinflammation and related disorders in a subject in need thereof, the method comprising administering to the subject an effective amount of a SHP1 inhibitor, e.g., an allosteric SHP1 inhibitor, or a pharmaceutical composition thereof.

In an embodiment, the methods described herein provide a method of treating or preventing neuroinflammation in a subject in need thereof, the method comprising administering to the subject an effective amount of a SHP1 inhibitor, e.g., a SHP1 inhibitor described herein.

In an embodiment, the methods described herein provide a method of treating or preventing a neuroinflammatory disorder in a subject in need thereof, the method comprising administering to the subject an effective amount of a SHP1 inhibitor, e.g., a SHP1 inhibitor described herein.

In an embodiment, the methods described herein provide a method of treating or preventing a disorder associated with neuroinflammation in a subject in need thereof, the method comprising administering to the subject an effective amount of a SHP1 inhibitor, e.g., a SHP1 inhibitor described herein. In some embodiments, the neuroinflammation is associated with a separate disorder in the subject.

In an embodiment, the methods described herein provide a method of treating or preventing Multiple Sclerosis, Parkinson's disease, Multiple System Atrophy, Corticobasal Degeneration, Progressive Supranuclear Paresis, Guillain-Barre Syndrome (GBS), chronic inflammatory demyelinating polyneuropathy (CIDP), viral encephalitis, cerebrovascular accidents, or cranial trauma in a subject in need thereof, comprising administering to the subject an effective amount of a SHP1 inhibitor, e.g., a SHP1 inhibitor described herein. In some embodiments, the Multiple Sclerosis is Relapse Remitting Multiple Sclerosis, Secondary Progressive Multiple Sclerosis or Primary Progressive Multiple Sclerosis.

Additionally described herein are other methods including a method of treating or preventing genetic disorders such as Noonan syndrome and Leopard syndrome, genetic disorders resulting in loss-of-function in the ras signaling pathway including Legius syndrome, neurodegenerative diseases such as Alzheimer's disease, Parkinson's disease, dementia with Lewy bodies, multiple system atrophy, Huntington's disease, spinocerebellar ataxias (e.g., SCA1, SCA2, SCA3, SCA6, SCAT, and SCA17), spinobulbar muscular atrophy (SBMA) or Kennedy disease, dentatorubropallidoluysian atrophy (DRPLA), ALS, AIDS dementia, frontotemporal dementia, corticobasal ganglionic degeneration, progressive supranuclear palsy, Creutzfeldt-Jakob disease, Gerstmann-Straussler-Scheinker syndrome, fatal familial insomnia, corticobasal ganglionic degeneration, hereditary spastic paraplegia, multiple sclerosis, neuromyelitis optica (Devic's disease), concentric sclerosis (Baló's disease), encephalomyelitis including acute disseminated encephalomyelitis (ADEM), acute haemorrhagic leucoencephalitis (AHL), Guillain-Barre Syndrome, chronic inflammatory demyelinating polyneuropathy (CIDP), transverse myelitis, Schilder's disease, fibromyalgia, and optic neuritis; demyelination due to injury such as spinal cord injury, traumatic brain injury, cerebral palsy, neuropathy (e.g. neuropathy due to diabetes, chronic renal failure, hypothyroidism, liver failure, or compression of the nerve (e.g. in Bell's palsy) and post radiation injury; demyelination due to hypoxic-ischaemic events such as stroke, acute ischemic optic neuropathy, or other ischemia, and carbon monoxide exposure; demyelination due to metabolic disruption such as central pontine myelolysis (CPM) or extrapontine myelinolysis (EPM); demyelination due to inherited conditions such as Charcot-Marie-Tooth disease (CMT), Sjogren-Larsson syndrome, Refsum disease, Krabbe disease, Canavan disease, Alexander disease, Friedreich's ataxia, Pelizaeus-Merzbacher disease, Bassen-Kornzweig syndrome, metachromatic leukodystrophy (MLD), adrenoleukodystrophy, Leber's optic neuropathy, and nerve damage due to pernicious anemia; demyelination due to a viral infection such as progressive multifocal leukoencephalopathy (PML), Lyme disease, tabes dorsalis due to untreated syphilis, HIV, or subacute sclerosing panencephalitis due to measles virus; demyelination due to toxic exposure such as chronic alcoholism (which is a possible cause of Marchiafava-Bignami disease), chemotherapy, mitochondrial toxins such as cyanide or hydrogen sulphide, or exposure to chemicals such as organophosphates; demyelination due to a dietary deficiency such as vitamin B12 deficiency, vitamin E deficiency and copper deficiency; or demyelination which has unknown causes or multiple causes such as trigeminal neuralgia, Marchiafava-Bignami disease, and Bell's palsy in a subject in need thereof, comprising administering to the subject an effective amount of a SHP1 inhibitor, e.g., a SHP1 inhibitor described herein.

In an embodiment, described herein is a method of treating neuroinflammation in a subject in need thereof, the method comprising administering to the subject a therapeutically effective amount of a SHP1 inhibitor, e.g., a SHP1 inhibitor described herein.

In an embodiment, described herein is a method of treating a neuroinflammatory disorder in a subject in need thereof, the method comprising administering to the subject a therapeutically effective amount of a SHP1 inhibitor, e.g., a SHP1 inhibitor described herein.

In an embodiment, described herein is a method of treating a disorder associated with neuroinflammation in a subject in need thereof, the method comprising administering to the subject a therapeutically effective amount of a SHP1 inhibitor, e.g., a SHP1 inhibitor described herein. In some embodiments, the neuroinflammation is associated with a separate disorder in the subject.

In an embodiment, the methods described herein provide a method of treating Multiple Sclerosis, Parkinson's disease, Multiple System Atrophy, Corticobasal Degeneration, Progressive Supranuclear Paresis, Guillain-Barre Syndrome (GBS), chronic inflammatory demyelinating polyneuropathy (CIDP), viral encephalitis, cerebrovascular accidents, or cranial trauma in a subject in need thereof, comprising administering to the subject a therapeutically effective amount of a SHP1 inhibitor, e.g., a SHP1 inhibitor described herein. In some embodiments, the Multiple Sclerosis is Relapse Remitting Multiple Sclerosis, Secondary Progressive Multiple Sclerosis or Primary Progressive Multiple Sclerosis.

Additionally described herein are other methods including a method of treating genetic disorders such as Noonan syndrome and Leopard syndrome, genetic disorders resulting in loss-of-function in the ras signaling pathway including Legius syndrome, neurodegenerative diseases such as Alzheimer's disease, Parkinson's disease, dementia with Lewy bodies, multiple system atrophy, Huntington's disease, spinocerebellar ataxias (e.g., SCA1, SCA2, SCA3, SCA6, SCAT, and SCA17), spinobulbar muscular atrophy (SBMA) or Kennedy disease, dentatorubropallidoluysian atrophy (DRPLA), ALS, AIDS dementia, frontotemporal dementia, corticobasal ganglionic degeneration, progressive supranuclear palsy, Creutzfeldt-Jakob disease, Gerstmann-Straussler-Scheinker syndrome, fatal familial insomnia, corticobasal ganglionic degeneration, hereditary spastic paraplegia, multiple sclerosis, neuromyelitis optica (Devic's disease), concentric sclerosis (Baló's disease), encephalomyelitis including acute disseminated encephalomyelitis (ADEM), acute haemorrhagic leucoencephalitis (AHL), Guillain-Barre Syndrome, chronic inflammatory demyelinating polyneuropathy (CIDP), transverse myelitis, Schilder's disease, fibromyalgia, and optic neuritis; demyelination due to injury such as spinal cord injury, traumatic brain injury, cerebral palsy, neuropathy (e.g. neuropathy due to diabetes, chronic renal failure, hypothyroidism, liver failure, or compression of the nerve (e.g. in Bell's palsy) and post radiation injury; demyelination due to hypoxic-ischaemic events such as stroke, acute ischemic optic neuropathy, or other ischemia, and carbon monoxide exposure; demyelination due to metabolic disruption such as central pontine myelolysis (CPM) or extrapontine myelinolysis (EPM); demyelination due to inherited conditions such as Charcot-Marie-Tooth disease (CMT), Sjogren-Larsson syndrome, Refsum disease, Krabbe disease, Canavan disease, Alexander disease, Friedreich's ataxia, Pelizaeus-Merzbacher disease, Bassen-Kornzweig syndrome, metachromatic leukodystrophy (MLD), adrenoleukodystrophy, Leber's optic neuropathy, and nerve damage due to pernicious anemia; demyelination due to a viral infection such as progressive multifocal leukoencephalopathy (PML), Lyme disease, tabes dorsalis due to untreated syphilis, HIV, or subacute sclerosing panencephalitis due to measles virus; demyelination due to toxic exposure such as chronic alcoholism (which is a possible cause of Marchiafava-Bignami disease), chemotherapy, mitochondrial toxins such as cyanide or hydrogen sulphide, or exposure to chemicals such as organophosphates; demyelination due to a dietary deficiency such as vitamin B12 deficiency, vitamin E deficiency and copper deficiency; or demyelination which has unknown causes or multiple causes such as trigeminal neuralgia, Marchiafava-Bignami disease, and Bell's palsy in a subject in need thereof, comprising administering to the subject a therapeutically effective amount of a SHP1 inhibitor, e.g., a SHP1 inhibitor described herein.

In some embodiments of the methods described herein, the SHP1 inhibitor is administered orally, parenterally, rectally, transdermally, intradermally, intrathecally, subcutaneously, intravenously, intramuscularly, or intranasally. In some embodiments, the SHP1 inhibitor is orally administered.

In another embodiment, provided herein is a method of treating a lysosomal storage disorder, e.g., a lysosomal storage disorder described herein, in a subject in need thereof, comprising administering to the subject a therapeutically effective amount of a compound described herein, e.g., a SHP1 inhibitor or SHP2 inhibitor described herein, or a pharmaceutically acceptable salt thereof.

In some embodiments, exemplary disorders that are treated by administering the compounds described herein, e.g., a SHP1 inhibitor or SHP2 inhibitor, include but are not limited to: Alzheimer's disease, Parkinson's disease, dementia with Lewy bodies, multiple system atrophy, Huntington's disease, lysosomal storage diseases including Gaucher, spinocerebellar ataxias (e.g., SCA1, SCA2, SCA3, SCA6, SCAT, and SCA17), spinobulbar muscular atrophy (SBMA) or Kennedy disease, dentatorubropallidoluysian atrophy (DRPLA), ALS, AIDS dementia, frontotemporal dementia, corticobasal ganglionic degeneration, progressive supranuclear palsy, Creutzfeldt-Jakob disease, Gerstmann-Straussler-Scheinker syndrome, fatal familial insomnia, corticobasal ganglionic degeneration, hereditary spastic paraplegia, multiple sclerosis, neuromyelitis optica (Devic's disease), concentric sclerosis (Baló's disease), encephalomyelitis including acute disseminated encephalomyelitis (ADEM), acute haemorrhagic leucoencephalitis (AHL), Guillain-Barre Syndrome, chronic inflammatory demyelinating polyneuropathy (CIDP), transverse myelitis, Schilder's disease, fibromyalgia, and optic neuritis; demyelination due to injury such as spinal cord injury, traumatic brain injury, cerebral palsy, neuropathy (e.g. neuropathy due to diabetes, chronic renal failure, hypothyroidism, liver failure, or compression of the nerve (e.g. in Bell's palsy) and post radiation injury; demyelination due to hypoxic-ischaemic events such as stroke, acute ischemic optic neuropathy, or other ischemia, and carbon monoxide exposure; demyelination due to metabolic disruption such as central pontine myelolysis (CPM) or extrapontine myelinolysis (EPM); demyelination due to inherited conditions such as Charcot-Marie-Tooth disease (CMT), Sjogren-Larsson syndrome, Refsum disease, Krabbe disease, Canavan disease, Alexander disease, Friedreich's ataxia, Pelizaeus-Merzbacher disease, Bassen-Kornzweig syndrome, metachromatic leukodystrophy (MLD), adrenoleukodystrophy, Leber's optic neuropathy, and nerve damage due to pernicious anemia; demyelination due to a viral infection such as progressive multifocal leukoencephalopathy (PML), Lyme disease, tabes dorsalis due to untreated syphilis, HIV, or subacute sclerosing panencephalitis due to measles virus; demyelination due to toxic exposure such as chronic alcoholism (which is a possible cause of Marchiafava-Bignami disease), chemotherapy, mitochondrial toxins such as cyanide or hydrogen sulphide, or exposure to chemicals such as organophosphates; demyelination due to a dietary deficiency such as vitamin B12 deficiency, vitamin E deficiency and copper deficiency; or demyelination which has unknown causes or multiple causes such as trigeminal neuralgia, Marchiafava-Bignami disease, and Bell's palsy.

Exemplary disorders that can be treated by the methods described herein include lysosomal storage diseases and related disorders. Accordingly, in some embodiments, exemplary disorders that can be treated by the compounds described herein, e.g., a SHP1 inhibitor or SHP2 inhibitor described herein, include but are not limited to lysosomal storage diseases, such as Activator deficiency, Alpha-mannosidosis, AB variant, Aspartylglucosaminuria, Batten-Spielmeyer-Vogt disease, Beta-galactosidase/GM1 gangliosidosis, Beta-mannosidosis, Chronic hexosaminidase A deficiency, cystinosis, CLN7 disease, Congenital cathepsin D deficiency, Cholesteryl ester storage disease, Cystinosis, Danon disease, Fabry disease, fucosidosis, I-cell disease, Krabbe disease, Farber disease, Finnish Variant, gangliosidosis, galactosialidosis, Gaucher disease (including type I, type II, and type III), GM2-AP deficiency, glycoprotein storage disorders, glucocerebroside, glycogen storage disease type II (pompe disease), GM2 gangliosidosis, German/Serbian late infantile, Hunter syndrome, Hurler syndrome, Hurler-Scheie syndrome, hyaluronidase deficiency, Infantile free sialic acid storage disease, Jansky-Bielschowsky disease, Juvenile hexosaminidase A deficiency, Late infantile variant, leukodystrophies, Kufs disease, lysosomal acid lipase deficiency, Maroteaux-Lamy syndrome, Metachromatic leukodystrophy, metachromatic leukodystrophy, Morquio syndrome, multiple sulfatase deficiency, multiple sulfatase deficiency, mucopolysaccharidoses, Mucolipidosis Type I (sialidosis), Mucolipidosis Type II (I-cell disease), Mucolipidosis Type III (pseudo-Hurler polydystrophy/phosphotransferase deficiency), Mucolipidosis Type IV (mucolipidin 1 deficiency), Niemann-Pick diseases, Neuronal ceroid lipofuscinoses, Northern epilepsy, Pycnodysostosis, Pompe disease, Salla disease, SAP deficiency, Sandhoff disease, saposin B deficiency, Sanfilippo syndrome, Santavuori-Haltia disease, Scheie syndrome, Schindler disease, Sly syndrome, Sphingolipidoses, sulfatidosis, Tay-Sachs disease, Turkish late infantile, variant AB, and Wolman disease.

### Methods of Treatment Definitions

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition. In an alternate embodiment, the present invention contemplates administration of the compounds of the present invention as a prophylactic before a subject begins to suffer from the specified disease, disorder or condition.

In general, the “effective amount” of a compound refers to an amount sufficient to elicit the desired biological response, e.g., to treat a CNS-related disorder, is sufficient to induce anesthesia or sedation. As will be appreciated by those of ordinary skill in this art, the effective amount of a compound of the invention may vary depending on such factors as the desired biological endpoint, the pharmacokinetics of the compound, the disease being treated, the mode of administration, and the age, weight, health, and condition of the subject. An effective amount encompasses therapeutic and prophylactic treatment.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

A “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

The terms “subject” and “patient” are used interchangeably herein.

### Pharmaceutical Compositions

In one aspect, provided herein is a pharmaceutical composition comprising a compound described herein, e.g., a SHP2 inhibitor described herein, or a pharmaceutically acceptable salt thereof, and a pharmaceutically acceptable excipient. In certain embodiments, the compound of the present invention is provided in an effective amount in the pharmaceutical composition. In certain embodiments, the compound of the present invention is provided in a therapeutically effective amount. In certain embodiments, the compound of the present invention is provided in a prophylactically effective amount.

In certain embodiments, the pharmaceutical composition comprises an effective amount of the active ingredient. In certain embodiments, the pharmaceutical composition comprises a therapeutically effective amount of the active ingredient. In certain embodiments, the pharmaceutical composition comprises a prophylactically effective amount of the active ingredient.

The pharmaceutical compositions provided herein can be administered by a variety of routes including, but not limited to, oral (enteral) administration, parenteral (by injection) administration, rectal administration, transdermal administration, intradermal administration, intrathecal administration, subcutaneous (SC) administration, intravenous (IV) administration, intramuscular (IM) administration, and intranasal administration.

Generally, the compounds provided herein are administered in an effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

When used to prevent the onset of a disorder, the compounds provided herein will be administered to a subject at risk for developing the condition, typically on the advice and under the supervision of a physician, at the dosage levels described above. Subjects at risk for developing a particular condition generally include those that have a family history of the condition, or those who have been identified by genetic testing or screening to be particularly susceptible to developing the condition. Genetic testing can comprise identification of mutations in PTPN11 or genes that affect signalling through the Ras-MAPK, PI3K/AKT, and/or JAK-STAT pathways.

The pharmaceutical compositions provided herein can also be administered chronically (“chronic administration”). Chronic administration refers to administration of a compound or pharmaceutical composition thereof over an extended period of time, e.g., for example, over 3 months, 6 months, 1 year, 2 years, 3 years, 5 years, etc, or may be continued indefinitely, for example, for the rest of the subject's life. In certain embodiments, the chronic administration is intended to provide a constant level of the compound in the blood, e.g., within the therapeutic window over the extended period of time.

The pharmaceutical compositions of the present invention may be further delivered using a variety of dosing methods. For example, in certain embodiments, the pharmaceutical composition may be given as a bolus, e.g., in order to raise the concentration of the compound in the blood to an effective level. The placement of the bolus dose depends on the systemic levels of the active ingredient desired throughout the body, e.g., an intramuscular or subcutaneous bolus dose allows a slow release of the active ingredient, while a bolus delivered directly to the veins (e.g., through an IV drip) allows a much faster delivery which quickly raises the concentration of the active ingredient in the blood to an effective level. In other embodiments, the pharmaceutical composition may be administered as a continuous infusion, e.g., by IV drip, to provide maintenance of a steady-state concentration of the active ingredient in the subject's body. Furthermore, in still yet other embodiments, the pharmaceutical composition may be administered as first as a bolus dose, followed by continuous infusion.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or preferably from about 1 to about 40% by weight) with the remainder being various vehicles or excipients and processing aids helpful for forming the desired dosing form.

With oral dosing, one to five and especially two to four and typically three oral doses per day are representative regimens. Using these dosing patterns, each dose provides from about 0.01 to about 20 mg/kg of the compound provided herein, with preferred doses each providing from about 0.1 to about 10 mg/kg, and especially about 1 to about 5 mg/kg.

Transdermal doses are generally selected to provide similar or lower blood levels than are achieved using injection doses, generally in an amount ranging from about 0.01 to about 20% by weight, preferably from about 0.1 to about 20% by weight, preferably from about 0.1 to about 10% by weight, and more preferably from about 0.5 to about 15% by weight.

Injection dose levels range from about 0.1 mg/kg/hour to at least 20 mg/kg/hour, all for from about 1 to about 120 hours and especially 24 to 96 hours. A preloading bolus of from about 0.1 mg/kg to about 10 mg/kg or more may also be administered to achieve adequate steady state levels. The maximum total dose is not expected to exceed about 5 g/day for a 40 to 80 kg human patient.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable excipients known in the art. As before, the active compound in such compositions is typically a minor component, often being from about 0.05 to 10% by weight with the remainder being the injectable excipient and the like.

Transdermal compositions are typically formulated as a topical ointment or cream containing the active ingredient(s). When formulated as an ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or formulation. All such known transdermal formulations and ingredients are included within the scope provided herein.

The compounds provided herein can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pa., which is incorporated herein by reference.

The compounds of the present invention can also be administered in sustained release forms or from sustained release drug delivery systems. A description of representative sustained release materials can be found in Remington's Pharmaceutical Sciences.

The present invention also relates to the pharmaceutically acceptable acid addition salt of a compound of the present invention. The acid which may be used to prepare the pharmaceutically acceptable salt is that which forms a non-toxic acid addition salt, i.e., a salt containing pharmacologically acceptable anions such as the hydrochloride, hydroiodide, hydrobromide, nitrate, sulfate, bisulfate, phosphate, acetate, lactate, citrate, tartrate, succinate, maleate, fumarate, benzoate, para-toluenesulfonate, and the like.

In another aspect, the invention provides a pharmaceutical composition comprising a compound of the present invention and a pharmaceutically acceptable excipient, e.g., a composition suitable for injection, such as for intravenous (IV) administration.

Pharmaceutically acceptable excipients include any and all diluents or other liquid vehicles, dispersion or suspension aids, surface active agents, isotonic agents, preservatives, lubricants and the like, as suited to the particular dosage form desired, e.g., injection. General considerations in the formulation and/or manufacture of pharmaceutical compositions agents can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

For example, injectable preparations, such as sterile injectable aqueous suspensions, can be formulated according to the known art using suitable dispersing or wetting agents and suspending agents. Exemplary excipients that can be employed include, but are not limited to, water, sterile saline or phosphate-buffered saline, or Ringer's solution.

The injectable composition can be sterilized, for example, by filtration through a bacterial-retaining filter, or by incorporating sterilizing agents in the form of sterile solid compositions which can be dissolved or dispersed in sterile water or other sterile injectable medium prior to use.

Generally, the compounds provided herein are administered in an effective amount. The amount of the compound actually administered will typically be determined by a physician, in the light of the relevant circumstances, including the condition to be treated, the chosen route of administration, the actual compound administered, the age, weight, response of the individual patient, the severity of the patient's symptoms, and the like.

The compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include pre-filled, pre-measured ampules or syringes of the liquid compositions. In such compositions, the compound is usually a minor component (from about 0.1% to about 50% by weight or preferably from about 1% to about 40% by weight) with the remainder being various vehicles or carriers and processing aids helpful for forming the desired dosing form.

The compounds provided herein can be administered as the sole active agent, or they can be administered in combination with other active agents. In one aspect, the present invention provides a combination of a compound of the present invention and another pharmacologically active agent. Administration in combination can proceed by any technique apparent to those of skill in the art including, for example, separate, sequential, concurrent, and alternating administration.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation. General considerations in the formulation and/or manufacture of pharmaceutical compositions can be found, for example, in Remington: The Science and Practice of Pharmacy 21^{st} ed., Lippincott Williams & Wilkins, 2005.

### Chemical and Pharmaceutical Composition Definitions

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. The term “pharmaceutically acceptable cation” refers to an acceptable cationic counter-ion of an acidic functional group. Such cations are exemplified by sodium, potassium, calcium, magnesium, ammonium, tetraalkylammonium cations, and the like. See, e.g., Berge, et al., J. Pharm. Sci. (1977) 66(1): 1-79.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

### Abbreviations

MMP12: matrix metallopeptidase 12; IL-33: interleukin-33.

### Example 1. Dose-Dependent Reduction in Expression of Matrix Remodeling Genes and Proteins with Compound A

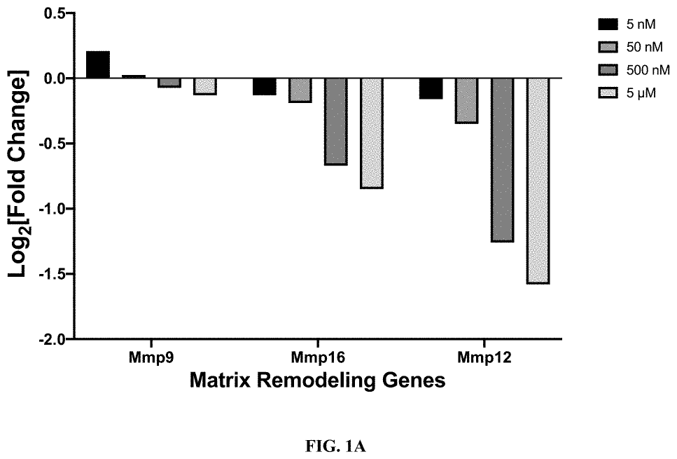

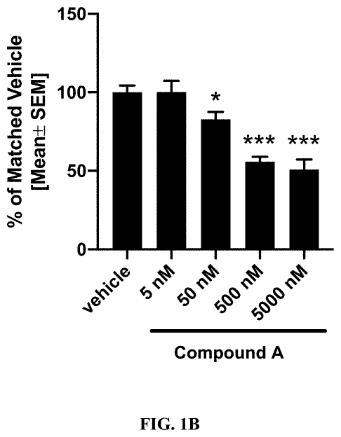

Dose-dependent reduction in expression of matrix remodeling genes and proteins with Compound A are shown in FIG. 1A and FIG. 1B. The treatment of organotypic cortical brain slice cultures from adult mice with Compound A for 48 h was associated with reductions in expression of matrix remodeling genes (mmp9, mmp16, mmp12; n=3-9, see FIG. 1A showing results of dosing of Compound A at 5 nM, 50 nM, 500 nM, and 5 μM) and total MMP12 protein (n=3-6, see FIG. 1B showing results of dosing of Compound A at 5 nM, 50 nM, 500 nM, and 5 μM). The observed reductions exhibited significant dose-dependence.

### Example 2. Dose-Dependent Reduction in Expression of Cytokine Genes and Proteins with Compound A

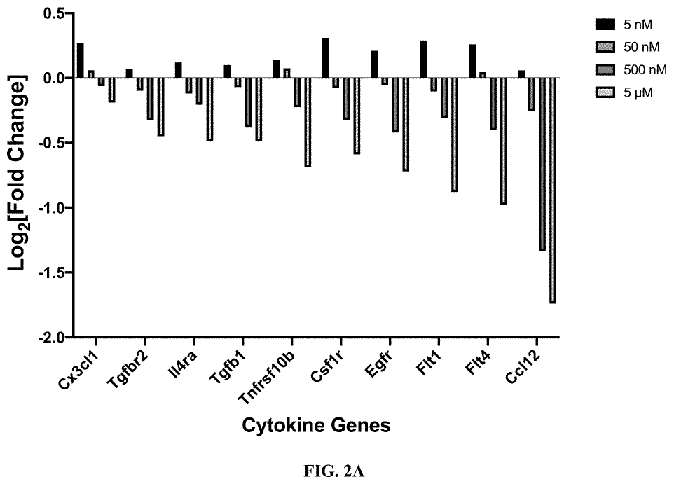

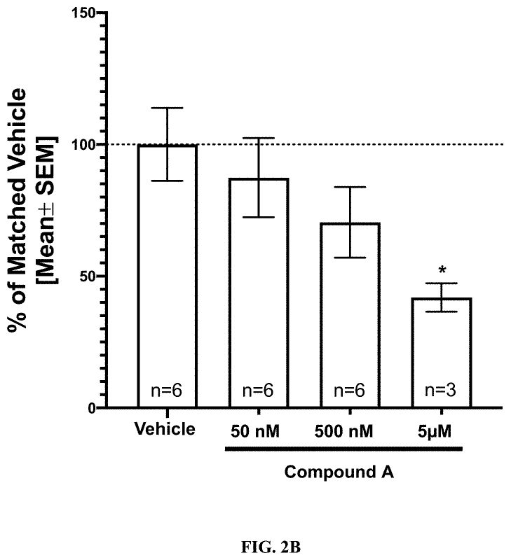

Dose-dependent reduction in expression of cytokine genes and proteins with Compound A are shown in FIG. 2A and FIG. 2B. The treatment of organotypic cortical slice cultures with Compound A for 48 h was associated with reductions in expression of cytokine genes (cx3cl1, tgfbr2, i14ra, tgfb1, tnfrsf10b, csf1r, egfr, fit1, flt4, ccl12; n=3-9, see FIG. 2A showing results of dosing of Compound A at 5 nM, 50 nM, 500 nM, and 5 μM) and pro-inflammatory cytokines like IL-33 (n=3-6, see FIG. 2B showing results of dosing of Compound A at 50 nM, 500 nM, and 5 μM).

### Example 3. Reduction in Expression of Cytokine Genes and Matrix Remodeling Genes with Compound B

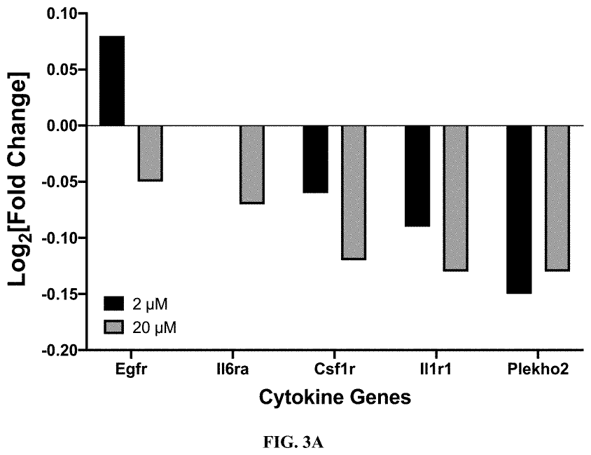

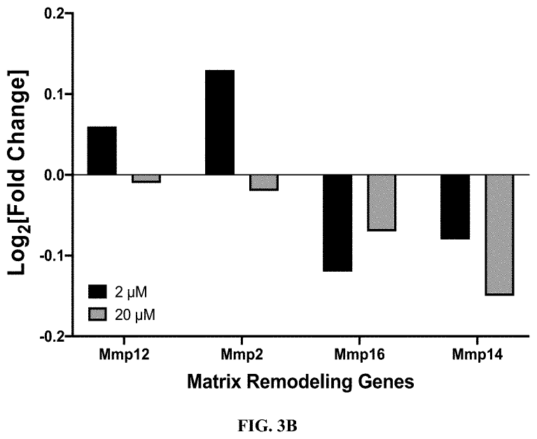

The treatment of organotypic cortical brain slice cultures from adult mice with Compound B for 48 h was associated with reductions in expression of cytokine genes (egfr, i16ra, csf1r, illr1, plekho2; n=3, see FIG. 3A showing results of dosing of Compound B at 2 μM, and 20 μM) and matrix remodeling genes (mmp12, mmp2, mmp16, mmp14; n=3, see FIG. 3B showing results of dosing of Compound B at 2 μM, and 20 μM).

### Example 4. Reduction in Expression of Astrocyte Marker Genes and Increases in Expression of Neuron Marker Genes with Compound B

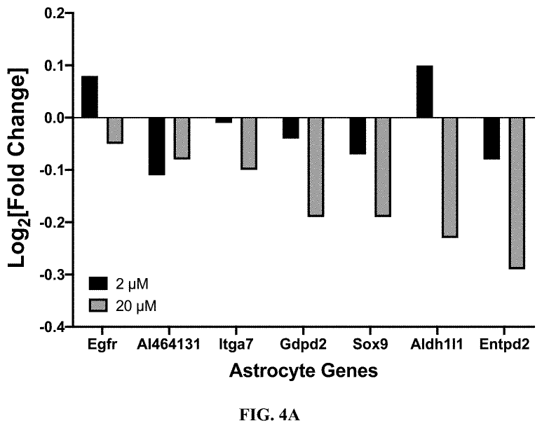

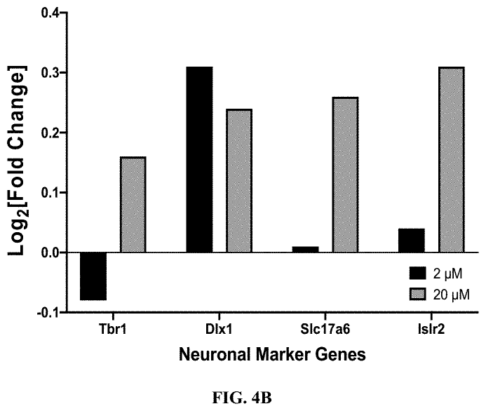

The treatment of organotypic cortical brain slice cultures from adult mice with Compound B for 48 h was associated with reductions in expression of astrocyte marker genes (egfr, AI464131, Itga7, Gdpd2, Sox9, Aldh1l1, Entpd2; n=3, see FIG. 4A showing results of dosing of Compound B at 2 μM, and 20 μM) and increases in expression of neuronal marker genes (tbr1, Dlx1, Slc17a6, Islr2; n=3, see FIG. 4B showing results of dosing of Compound B at 2 μM, and 20 μM).

### Example 5. Reduction in Expression of Cytokine Genes, Matrix Remodeling Genes, and Astrocyte Genes, and Increases in Expression of Neuronal Marker Genes with Compound C

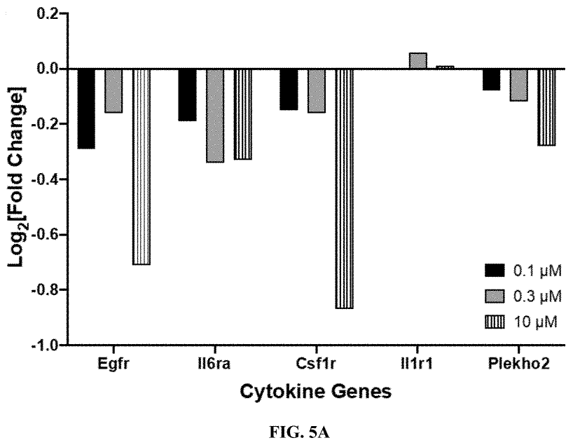

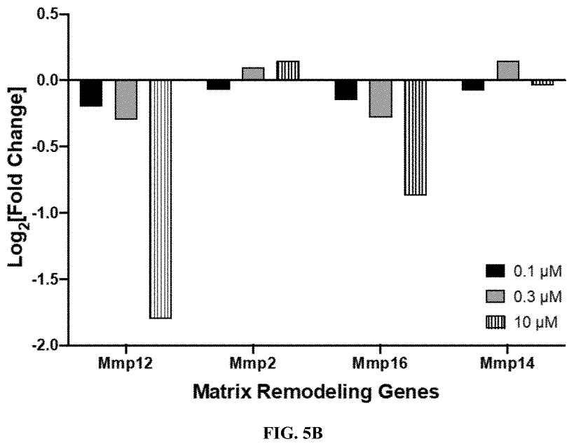

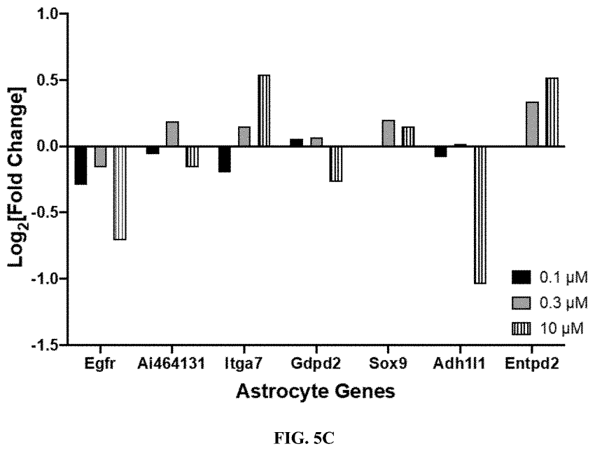

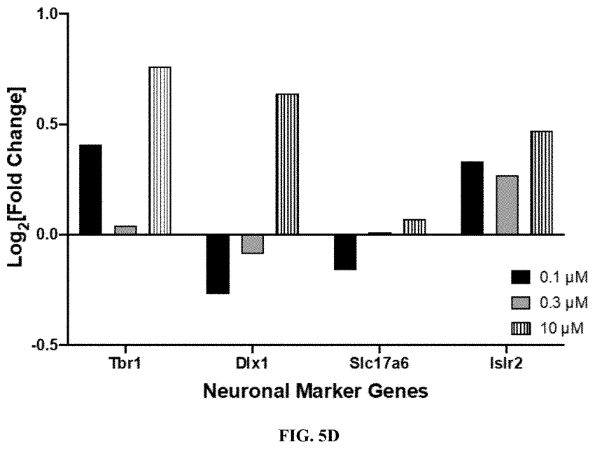

The treatment of organotypic brain mice cultures from adult mice with Compound C for 72 h was associated with reductions in expression of cytokine genes (egfr, i16ra, csf1r, plekho2; n=2, See FIG. 5A showing results of dosing of Compound C at 0.1 μM, 0.3 μM, and 10 μM), matrix remodeling genes (mmp12, mmp16; n=2, See FIG. 5B for results of dosing of compound C at 0.1 μM, 0.3 μM, and 10 μM), and astrocyte genes (egfr, ai464131, gdpd2, adh1l1; n=2, See FIG. 5C for results of dosing of compound C at 0.1 μM, 0.3 μM, and 10 μM), and increases in expression of neuronal marker genes (tbr1, dlx1, slc17a6, islr2; n=2, See FIG. 5D for results of dosing of compound C at 0.1 μM, 0.3 μM, and 10 μM).

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A method of treating or preventing neuroinflammation in a subject in need thereof, the method comprising administering to the subject an effective amount of a SHP2 inhibitor having the structure: 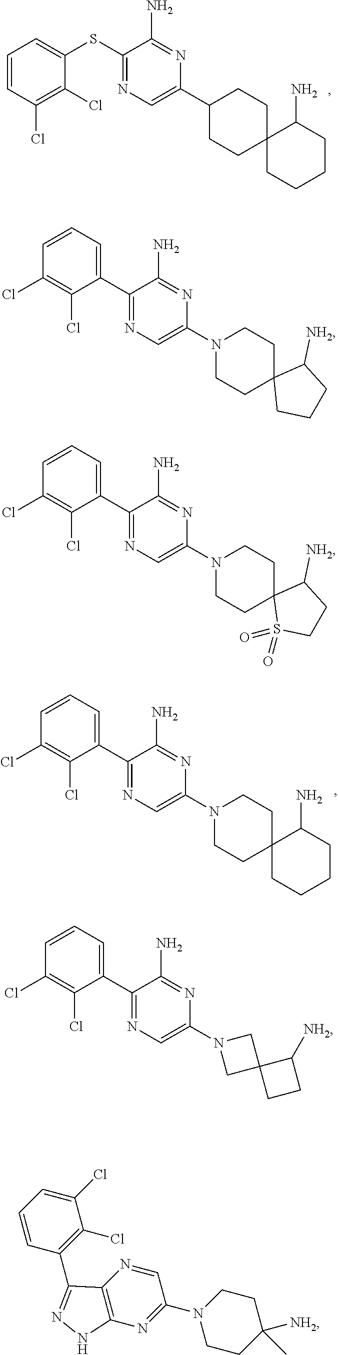 [SMILES: Nc1nc(C2CCC3(CCCCC3N)CC2)cnc1Sc1cccc(Cl)c1Cl ; Nc1nc(N2CCC3(CCCC3N)CC2)cnc1-c1cccc(Cl)c1Cl ; Nc1nc(N2CCC3(CC2)C(N)CCS3(=O)=O)cnc1-c1cccc(Cl)c1Cl ; Nc1nc(N2CCC3(CCCCC3N)CC2)cnc1-c1cccc(Cl)c1Cl ; Nc1nc(N2CC3(CCC3N)C2)cnc1-c1cccc(Cl)c1Cl ; CC1(N)CCN(c2cnc3c(-c4cccc(Cl)c4Cl)n[nH]c3n2)CC1] 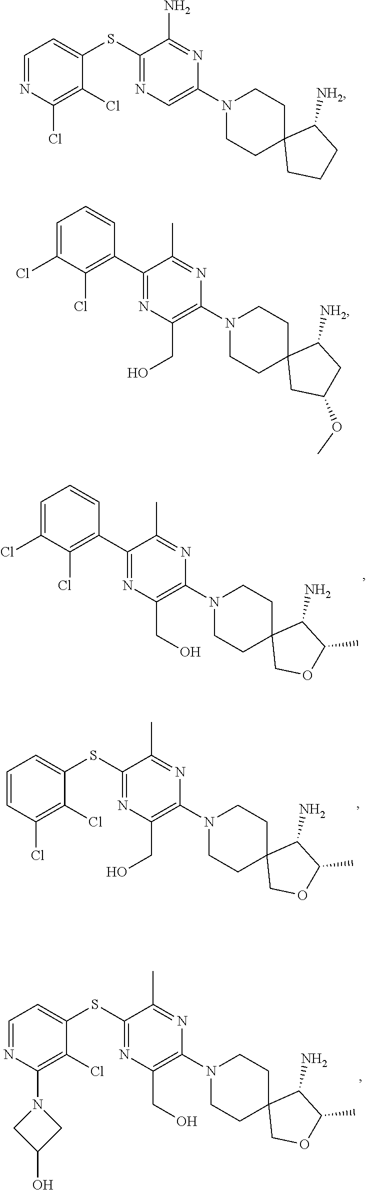 [SMILES: Nc1nc(N2CCC3(CCC[C@H]3N)CC2)cnc1Sc1ccnc(Cl)c1Cl ; CO[C@H]1C[C@@H](N)C2(CCN(c3nc(C)c(-c4cccc(Cl)c4Cl)nc3CO)CC2)C1 ; Cc1nc(N2CCC3(CC2)CO[C@@H](C)[C@H]3N)c(CO)nc1-c1cccc(Cl)c1Cl ; Cc1nc(N2CCC3(CC2)CO[C@@H](C)[C@H]3N)c(CO)nc1Sc1cccc(Cl)c1Cl ; Cc1nc(N2CCC3(CC2)CO[C@@H](C)[C@H]3N)c(CO)nc1Sc1ccnc(N2CC(O)C2)c1Cl] 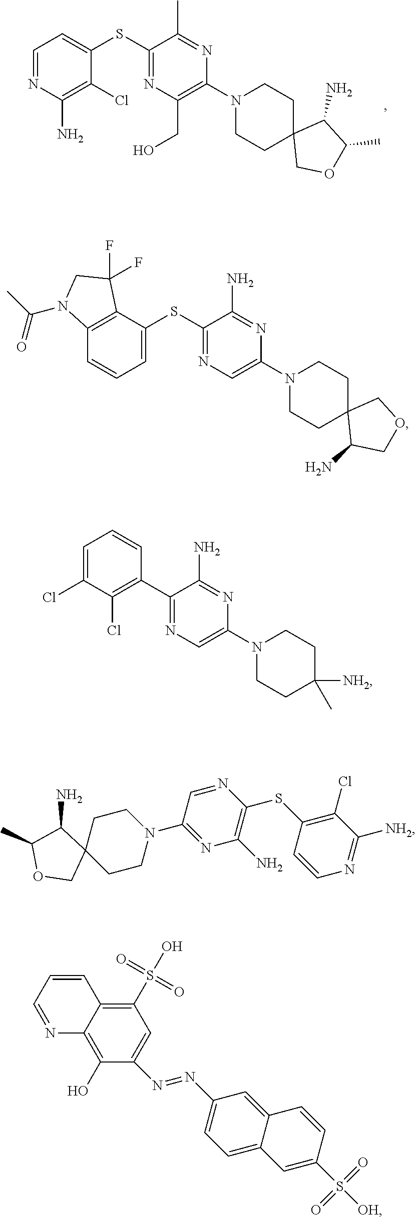
or a pharmaceutically acceptable salt thereof.

2. A method of treating or preventing a neuroinflammatory disorder in a subject in need thereof, the method comprising administering to the subject an effective amount of a SHP2 inhibitor having the structure: 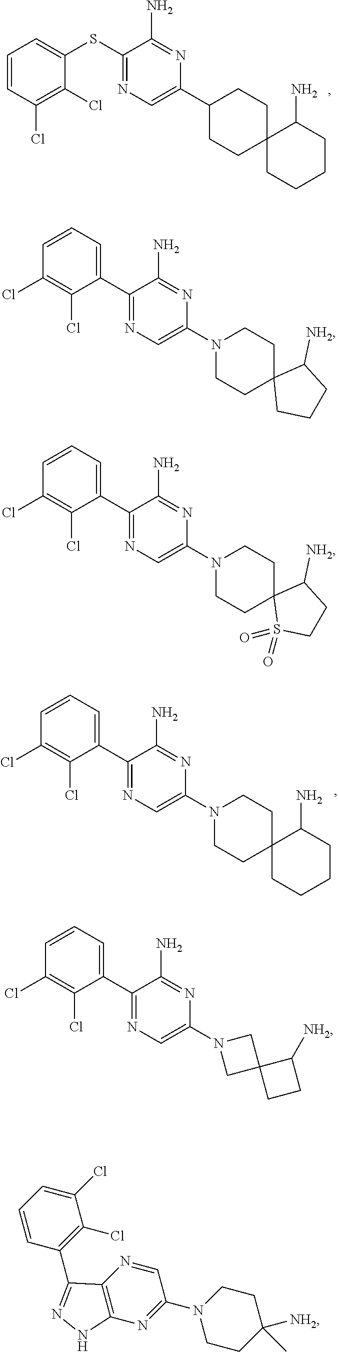 [SMILES: Nc1nc(C2CCC3(CCCCC3N)CC2)cnc1Sc1cccc(Cl)c1Cl ; Nc1nc(N2CCC3(CCCC3N)CC2)cnc1-c1cccc(Cl)c1Cl ; Nc1nc(N2CCC3(CC2)C(N)CCS3(=O)=O)cnc1-c1cccc(Cl)c1Cl ; Nc1nc(N2CCC3(CCCCC3N)CC2)cnc1-c1cccc(Cl)c1Cl ; Nc1nc(N2CC3(CCC3N)C2)cnc1-c1cccc(Cl)c1Cl ; CC1(N)CCN(c2cnc3c(-c4cccc(Cl)c4Cl)n[nH]c3n2)CC1] 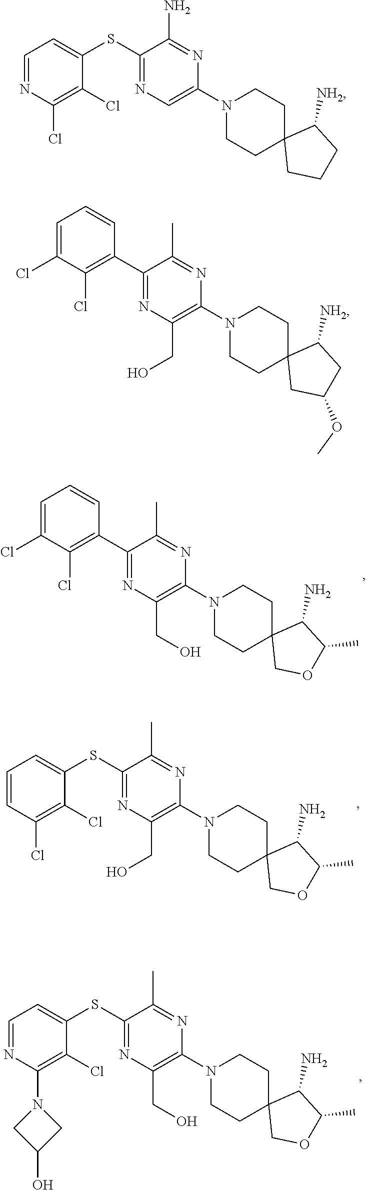 [SMILES: Nc1nc(N2CCC3(CCC[C@H]3N)CC2)cnc1Sc1ccnc(Cl)c1Cl ; CO[C@H]1C[C@@H](N)C2(CCN(c3nc(C)c(-c4cccc(Cl)c4Cl)nc3CO)CC2)C1 ; Cc1nc(N2CCC3(CC2)CO[C@@H](C)[C@H]3N)c(CO)nc1-c1cccc(Cl)c1Cl ; Cc1nc(N2CCC3(CC2)CO[C@@H](C)[C@H]3N)c(CO)nc1Sc1cccc(Cl)c1Cl ; Cc1nc(N2CCC3(CC2)CO[C@@H](C)[C@H]3N)c(CO)nc1Sc1ccnc(N2CC(O)C2)c1Cl] 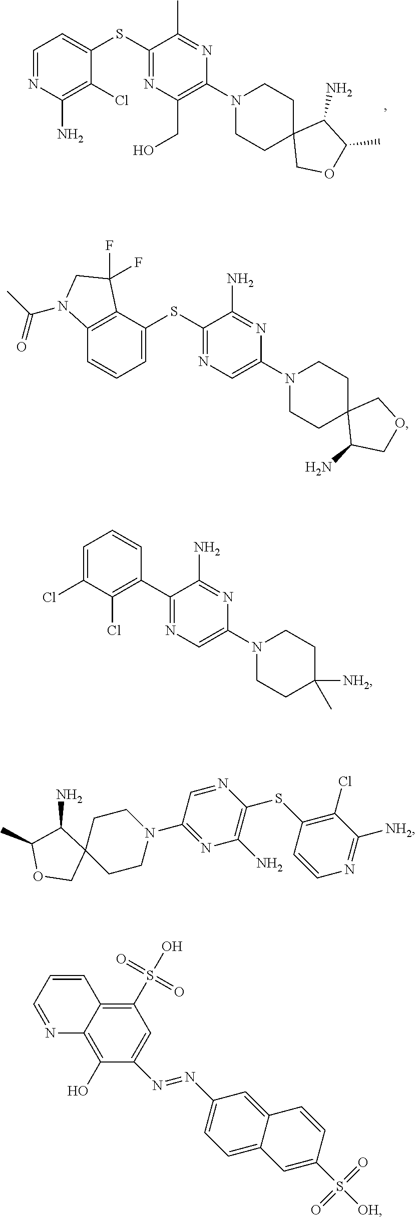
or a pharmaceutically acceptable salt thereof.

3. A method of treating or preventing a disorder associated with neuroinflammation in a subject in need thereof, the method comprising administering to the subject an effective amount of a SHP2 inhibitor having the structure: 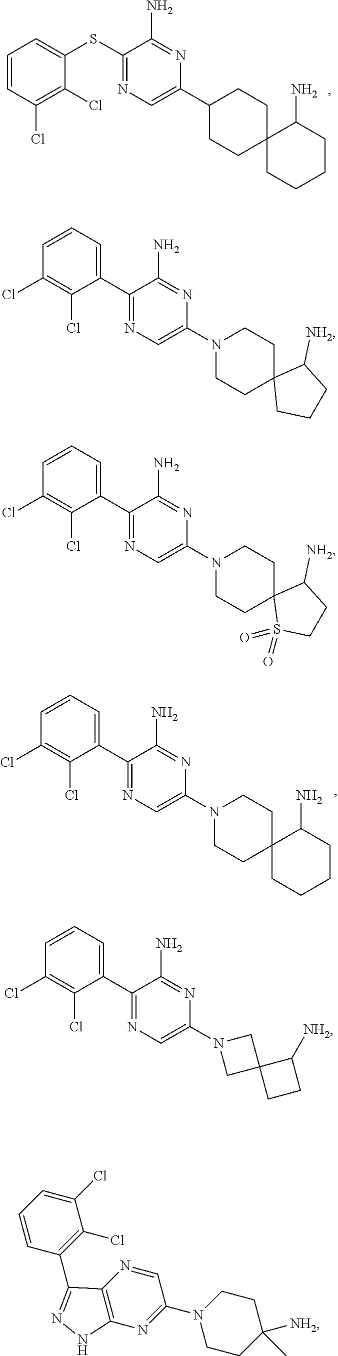 [SMILES: Nc1nc(C2CCC3(CCCCC3N)CC2)cnc1Sc1cccc(Cl)c1Cl ; Nc1nc(N2CCC3(CCCC3N)CC2)cnc1-c1cccc(Cl)c1Cl ; Nc1nc(N2CCC3(CC2)C(N)CCS3(=O)=O)cnc1-c1cccc(Cl)c1Cl ; Nc1nc(N2CCC3(CCCCC3N)CC2)cnc1-c1cccc(Cl)c1Cl ; Nc1nc(N2CC3(CCC3N)C2)cnc1-c1cccc(Cl)c1Cl ; CC1(N)CCN(c2cnc3c(-c4cccc(Cl)c4Cl)n[nH]c3n2)CC1] 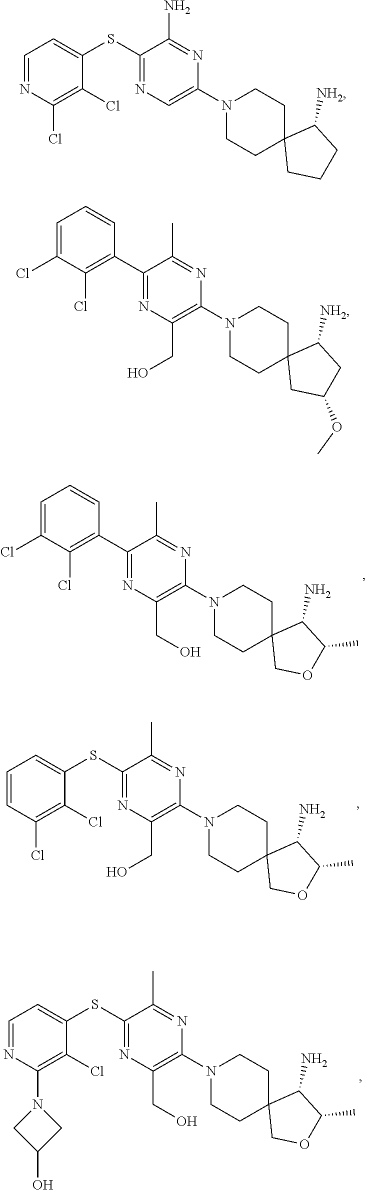 [SMILES: Nc1nc(N2CCC3(CCC[C@H]3N)CC2)cnc1Sc1ccnc(Cl)c1Cl ; CO[C@H]1C[C@@H](N)C2(CCN(c3nc(C)c(-c4cccc(Cl)c4Cl)nc3CO)CC2)C1 ; Cc1nc(N2CCC3(CC2)CO[C@@H](C)[C@H]3N)c(CO)nc1-c1cccc(Cl)c1Cl ; Cc1nc(N2CCC3(CC2)CO[C@@H](C)[C@H]3N)c(CO)nc1Sc1cccc(Cl)c1Cl ; Cc1nc(N2CCC3(CC2)CO[C@@H](C)[C@H]3N)c(CO)nc1Sc1ccnc(N2CC(O)C2)c1Cl] 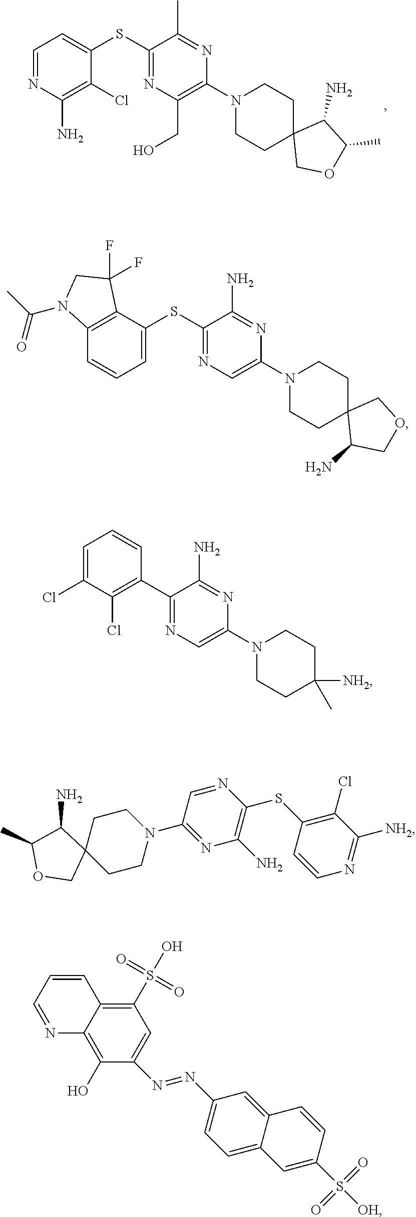
or a pharmaceutically acceptable salt thereof.

4. The method of claim 1, wherein the neuroinflammation is associated with a separate disorder in the subject.

5. A method of treating or preventing a neurodegenerative disorder in a subject in need thereof, comprising administering to the subject an effective amount of a SHP2 inhibitor having the structure: 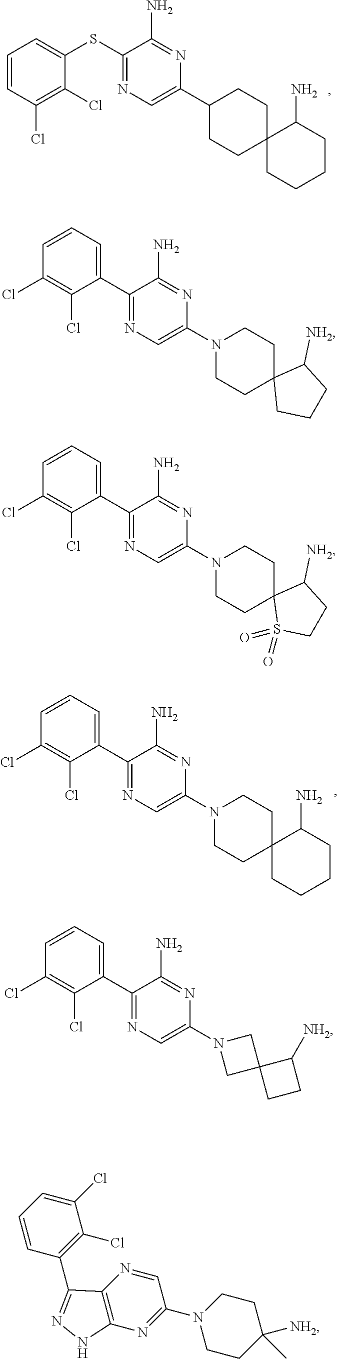 [SMILES: Nc1nc(C2CCC3(CCCCC3N)CC2)cnc1Sc1cccc(Cl)c1Cl ; Nc1nc(N2CCC3(CCCC3N)CC2)cnc1-c1cccc(Cl)c1Cl ; Nc1nc(N2CCC3(CC2)C(N)CCS3(=O)=O)cnc1-c1cccc(Cl)c1Cl ; Nc1nc(N2CCC3(CCCCC3N)CC2)cnc1-c1cccc(Cl)c1Cl ; Nc1nc(N2CC3(CCC3N)C2)cnc1-c1cccc(Cl)c1Cl ; CC1(N)CCN(c2cnc3c(-c4cccc(Cl)c4Cl)n[nH]c3n2)CC1] 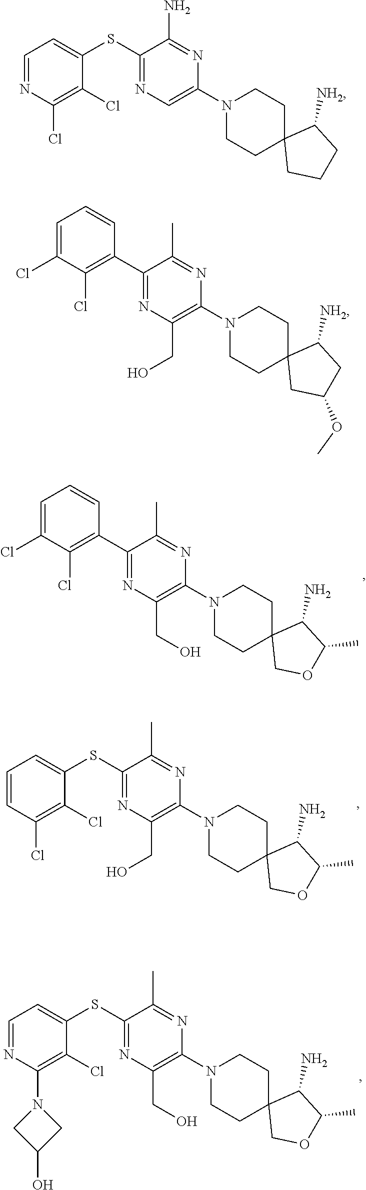 [SMILES: Nc1nc(N2CCC3(CCC[C@H]3N)CC2)cnc1Sc1ccnc(Cl)c1Cl ; CO[C@H]1C[C@@H](N)C2(CCN(c3nc(C)c(-c4cccc(Cl)c4Cl)nc3CO)CC2)C1 ; Cc1nc(N2CCC3(CC2)CO[C@@H](C)[C@H]3N)c(CO)nc1-c1cccc(Cl)c1Cl ; Cc1nc(N2CCC3(CC2)CO[C@@H](C)[C@H]3N)c(CO)nc1Sc1cccc(Cl)c1Cl ; Cc1nc(N2CCC3(CC2)CO[C@@H](C)[C@H]3N)c(CO)nc1Sc1ccnc(N2CC(O)C2)c1Cl] 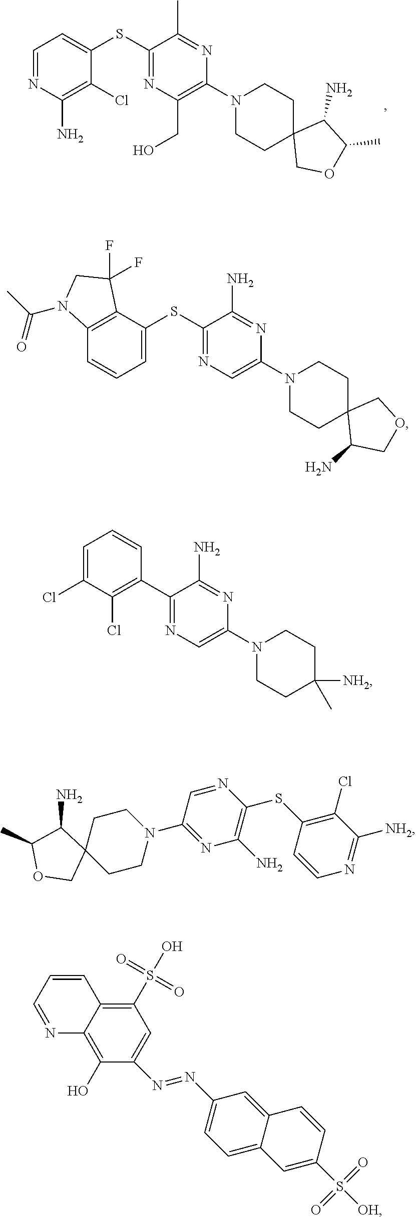
or a pharmaceutically acceptable salt thereof.

6. The method of claim 5, wherein the neurodegenerative disorder is Alzheimer's disease, Parkinson's disease, dementia with Lewy bodies, multiple system atrophy, Huntington's disease, spinocerebellar ataxias, spinobulbar muscular atrophy (SBMA) or Kennedy disease, dentatorubropallidoluysian atrophy (DRPLA), ALS, AIDS dementia, frontotemporal dementia, corticobasal ganglionic degeneration, progressive supranuclear palsy, Creutzfeldt-Jakob disease, Gerstmann-Straussler-Scheinker syndrome, fatal familial insomnia, corticobasal ganglionic degeneration, hereditary spastic paraplegia, multiple sclerosis, neuromyelitis optica (Devic's disease), concentric sclerosis (Baló's disease), encephalomyelitis including acute disseminated encephalomyelitis (ADEM), acute haemorrhagic leucoencephalitis (AHL), Guillain-Barre Syndrome, chronic inflammatory demyelinating polyneuropathy (CIDP), transverse myelitis, Schilder's disease, fibromyalgia, or optic neuritis.

7. A method of treating or preventing Multiple Sclerosis, Parkinson's disease, Multiple System Atrophy, Corticobasal Degeneration, Progressive Supranuclear Paresis, Guillain-Barre Syndrome (GBS), chronic inflammatory demyelinating polyneuropathy (CIDP), viral encephalitis, cerebrovascular accidents, or cranial trauma in a subject in need thereof, comprising administering to the subject an effective amount of a SHP2 inhibitor having the structure: 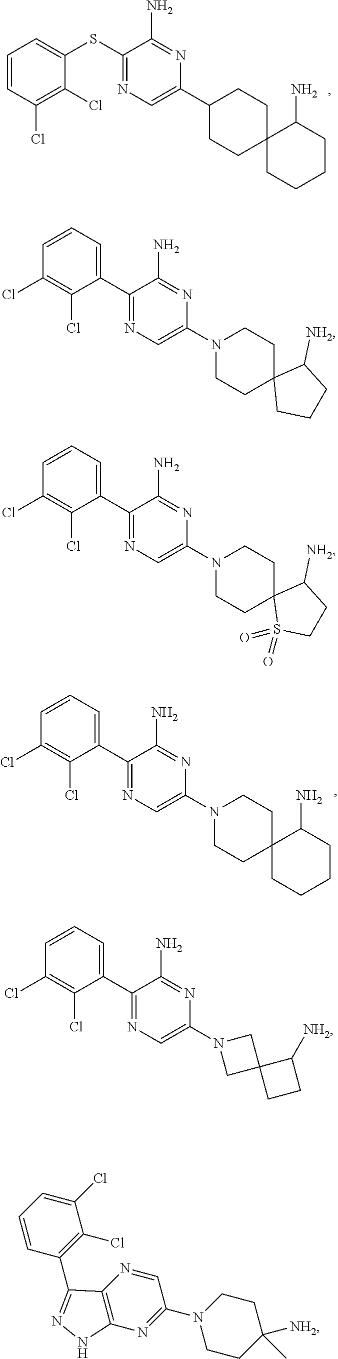 [SMILES: Nc1nc(C2CCC3(CCCCC3N)CC2)cnc1Sc1cccc(Cl)c1Cl ; Nc1nc(N2CCC3(CCCC3N)CC2)cnc1-c1cccc(Cl)c1Cl ; Nc1nc(N2CCC3(CC2)C(N)CCS3(=O)=O)cnc1-c1cccc(Cl)c1Cl ; Nc1nc(N2CCC3(CCCCC3N)CC2)cnc1-c1cccc(Cl)c1Cl ; Nc1nc(N2CC3(CCC3N)C2)cnc1-c1cccc(Cl)c1Cl ; CC1(N)CCN(c2cnc3c(-c4cccc(Cl)c4Cl)n[nH]c3n2)CC1] 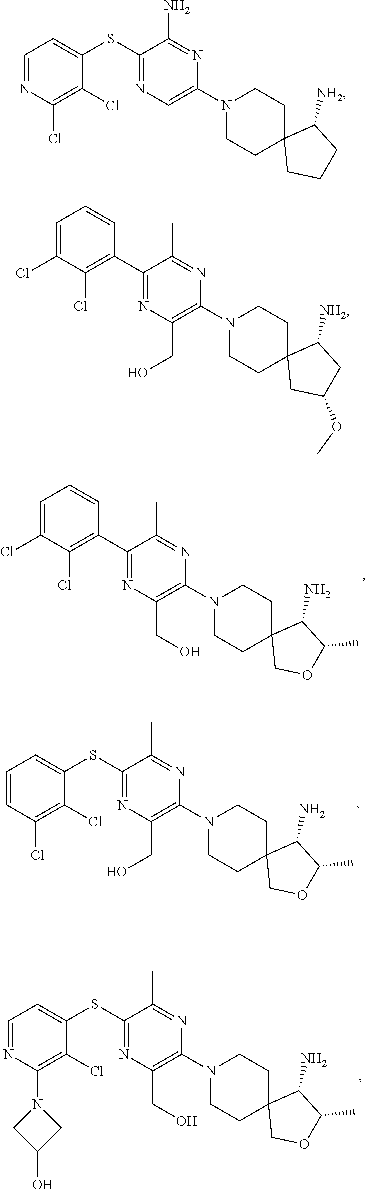 [SMILES: Nc1nc(N2CCC3(CCC[C@H]3N)CC2)cnc1Sc1ccnc(Cl)c1Cl ; CO[C@H]1C[C@@H](N)C2(CCN(c3nc(C)c(-c4cccc(Cl)c4Cl)nc3CO)CC2)C1 ; Cc1nc(N2CCC3(CC2)CO[C@@H](C)[C@H]3N)c(CO)nc1-c1cccc(Cl)c1Cl ; Cc1nc(N2CCC3(CC2)CO[C@@H](C)[C@H]3N)c(CO)nc1Sc1cccc(Cl)c1Cl ; Cc1nc(N2CCC3(CC2)CO[C@@H](C)[C@H]3N)c(CO)nc1Sc1ccnc(N2CC(O)C2)c1Cl] 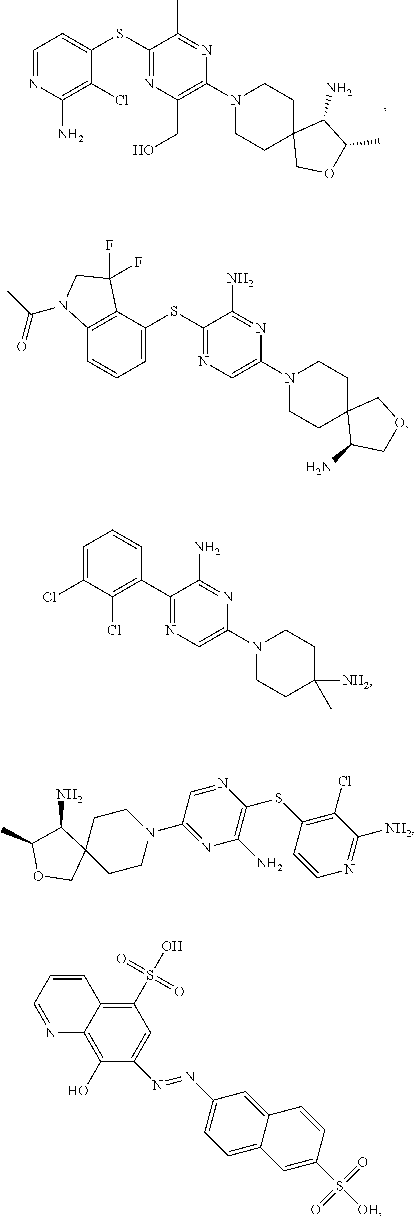
or a pharmaceutically acceptable salt thereof.

8. The method of claim 7, wherein the Multiple Sclerosis is Relapse Remitting Multiple Sclerosis, Secondary Progressive Multiple Sclerosis or Primary Progressive Multiple Sclerosis.

9. A method of treating or preventing a genetic disorder resulting in gain-of-function in SHP2 in a subject in need thereof, comprising administering to the subject an effective amount of a SHP2 inhibitor having the structure: 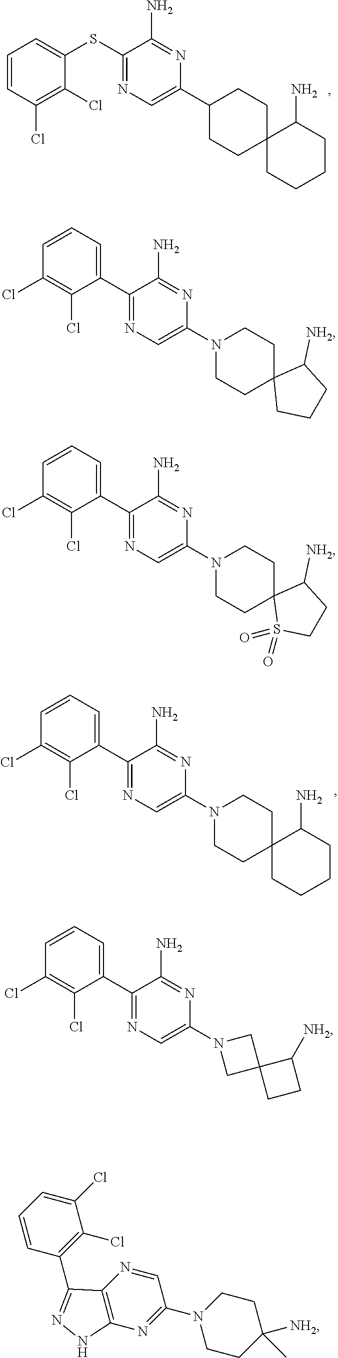 [SMILES: Nc1nc(C2CCC3(CCCCC3N)CC2)cnc1Sc1cccc(Cl)c1Cl ; Nc1nc(N2CCC3(CCCC3N)CC2)cnc1-c1cccc(Cl)c1Cl ; Nc1nc(N2CCC3(CC2)C(N)CCS3(=O)=O)cnc1-c1cccc(Cl)c1Cl ; Nc1nc(N2CCC3(CCCCC3N)CC2)cnc1-c1cccc(Cl)c1Cl ; Nc1nc(N2CC3(CCC3N)C2)cnc1-c1cccc(Cl)c1Cl ; CC1(N)CCN(c2cnc3c(-c4cccc(Cl)c4Cl)n[nH]c3n2)CC1] 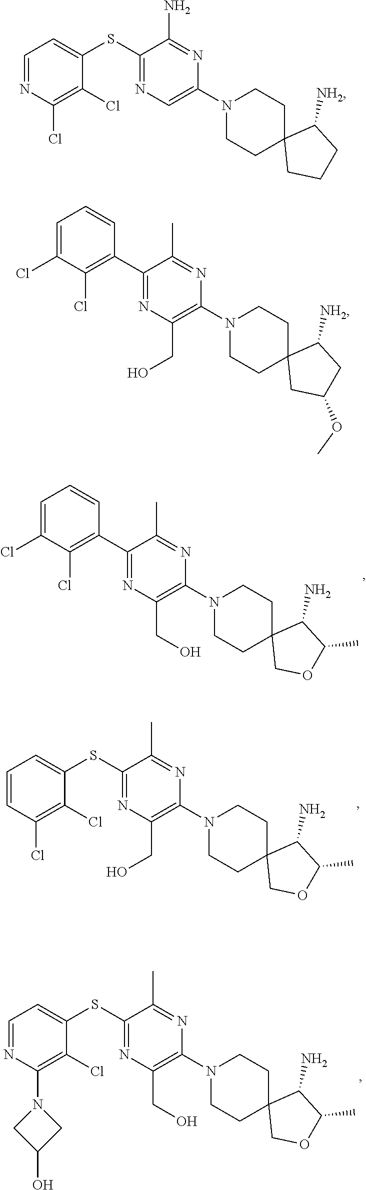 [SMILES: Nc1nc(N2CCC3(CCC[C@H]3N)CC2)cnc1Sc1ccnc(Cl)c1Cl ; CO[C@H]1C[C@@H](N)C2(CCN(c3nc(C)c(-c4cccc(Cl)c4Cl)nc3CO)CC2)C1 ; Cc1nc(N2CCC3(CC2)CO[C@@H](C)[C@H]3N)c(CO)nc1-c1cccc(Cl)c1Cl ; Cc1nc(N2CCC3(CC2)CO[C@@H](C)[C@H]3N)c(CO)nc1Sc1cccc(Cl)c1Cl ; Cc1nc(N2CCC3(CC2)CO[C@@H](C)[C@H]3N)c(CO)nc1Sc1ccnc(N2CC(O)C2)c1Cl] 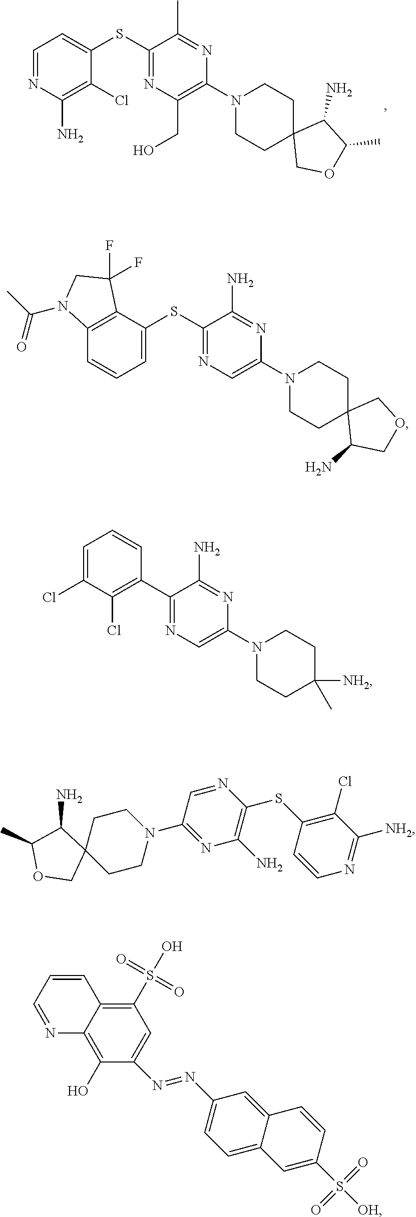
or a pharmaceutically acceptable salt thereof.

10. The method of claim 9, wherein the genetic disorder resulting in gain-of-function in SHP2 is Noonan syndrome or Leopard syndrome.

11. A method of treating or preventing a genetic disorder resulting in loss-of-function in the ras signaling pathway in a subject in need thereof, comprising administering to the subject an effective amount of a SHP2 inhibitor having the structure: 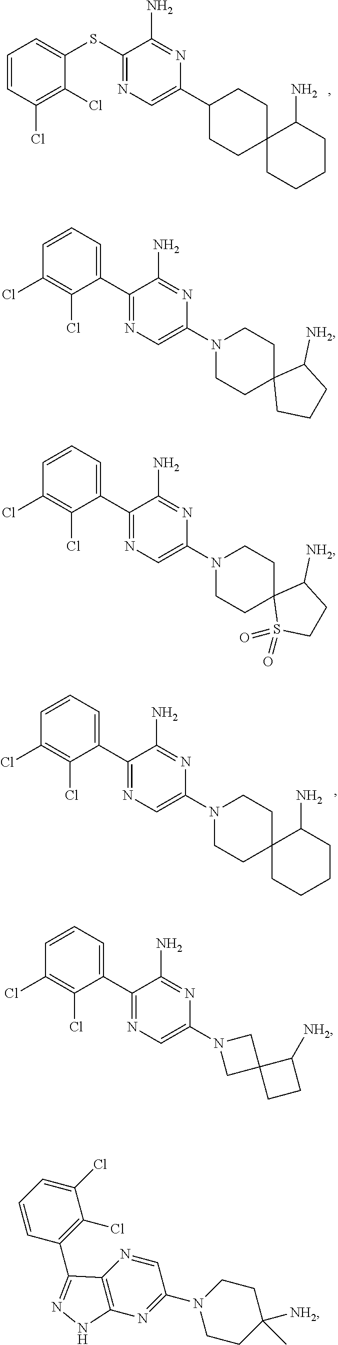 [SMILES: Nc1nc(C2CCC3(CCCCC3N)CC2)cnc1Sc1cccc(Cl)c1Cl ; Nc1nc(N2CCC3(CCCC3N)CC2)cnc1-c1cccc(Cl)c1Cl ; Nc1nc(N2CCC3(CC2)C(N)CCS3(=O)=O)cnc1-c1cccc(Cl)c1Cl ; Nc1nc(N2CCC3(CCCCC3N)CC2)cnc1-c1cccc(Cl)c1Cl ; Nc1nc(N2CC3(CCC3N)C2)cnc1-c1cccc(Cl)c1Cl ; CC1(N)CCN(c2cnc3c(-c4cccc(Cl)c4Cl)n[nH]c3n2)CC1] 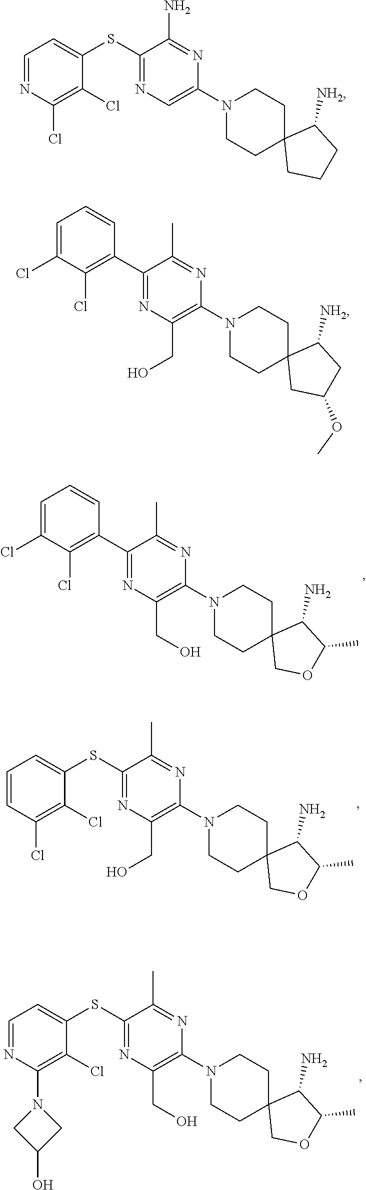 [SMILES: Nc1nc(N2CCC3(CCC[C@H]3N)CC2)cnc1Sc1ccnc(Cl)c1Cl ; CO[C@H]1C[C@@H](N)C2(CCN(c3nc(C)c(-c4cccc(Cl)c4Cl)nc3CO)CC2)C1 ; Cc1nc(N2CCC3(CC2)CO[C@@H](C)[C@H]3N)c(CO)nc1-c1cccc(Cl)c1Cl ; Cc1nc(N2CCC3(CC2)CO[C@@H](C)[C@H]3N)c(CO)nc1Sc1cccc(Cl)c1Cl ; Cc1nc(N2CCC3(CC2)CO[C@@H](C)[C@H]3N)c(CO)nc1Sc1ccnc(N2CC(O)C2)c1Cl] 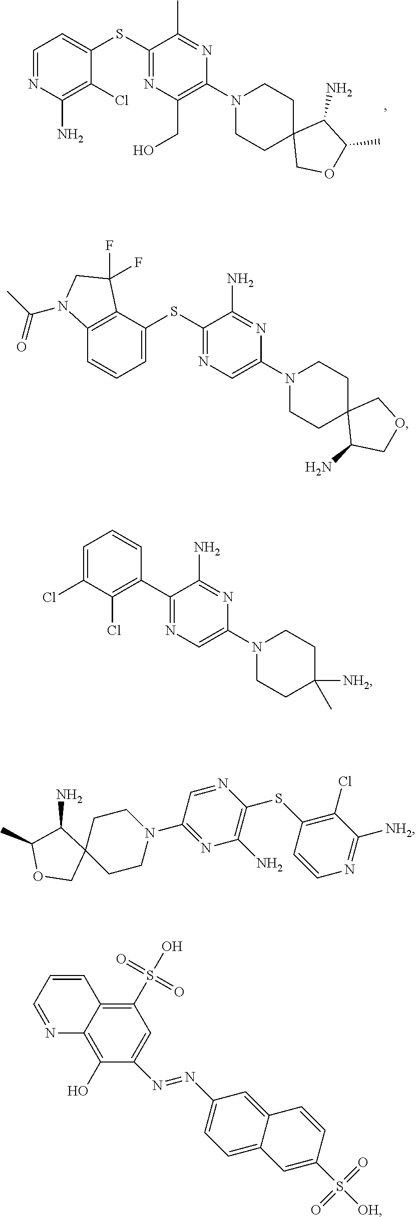
or a pharmaceutically acceptable salt thereof.

12. The method of claim 11, wherein the genetic disorder resulting in loss-of-function in the ras signaling pathway is Legius syndrome.

13. A method of treating or preventing demyelination in a subject in need thereof, comprising administering to the subject an effective amount of a SHP2 inhibitor having the structure: 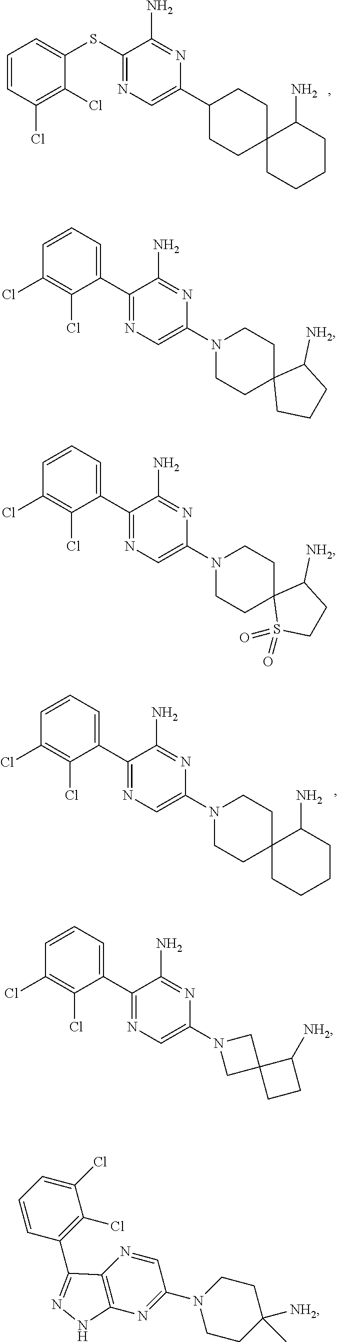 [SMILES: Nc1nc(C2CCC3(CCCCC3N)CC2)cnc1Sc1cccc(Cl)c1Cl ; Nc1nc(N2CCC3(CCCC3N)CC2)cnc1-c1cccc(Cl)c1Cl ; Nc1nc(N2CCC3(CC2)C(N)CCS3(=O)=O)cnc1-c1cccc(Cl)c1Cl ; Nc1nc(N2CCC3(CCCCC3N)CC2)cnc1-c1cccc(Cl)c1Cl ; Nc1nc(N2CC3(CCC3N)C2)cnc1-c1cccc(Cl)c1Cl ; CC1(N)CCN(c2cnc3c(-c4cccc(Cl)c4Cl)n[nH]c3n2)CC1] 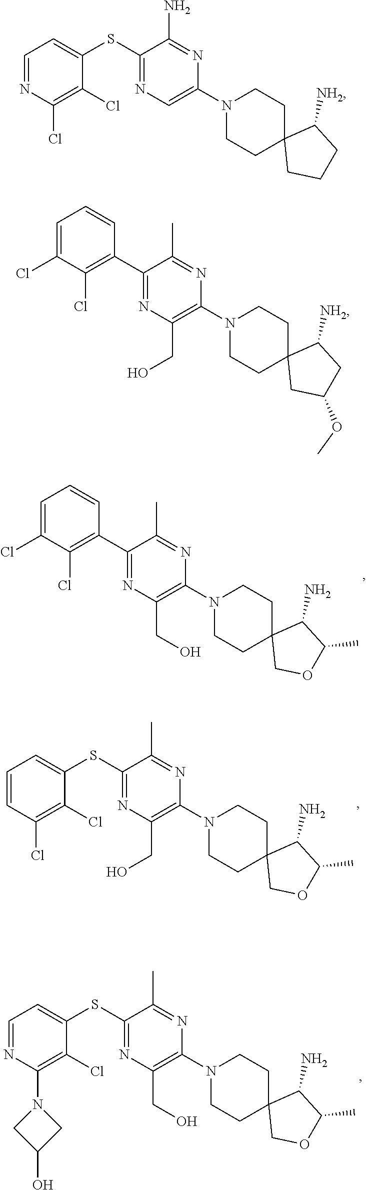 [SMILES: Nc1nc(N2CCC3(CCC[C@H]3N)CC2)cnc1Sc1ccnc(Cl)c1Cl ; CO[C@H]1C[C@@H](N)C2(CCN(c3nc(C)c(-c4cccc(Cl)c4Cl)nc3CO)CC2)C1 ; Cc1nc(N2CCC3(CC2)CO[C@@H](C)[C@H]3N)c(CO)nc1-c1cccc(Cl)c1Cl ; Cc1nc(N2CCC3(CC2)CO[C@@H](C)[C@H]3N)c(CO)nc1Sc1cccc(Cl)c1Cl ; Cc1nc(N2CCC3(CC2)CO[C@@H](C)[C@H]3N)c(CO)nc1Sc1ccnc(N2CC(O)C2)c1Cl] 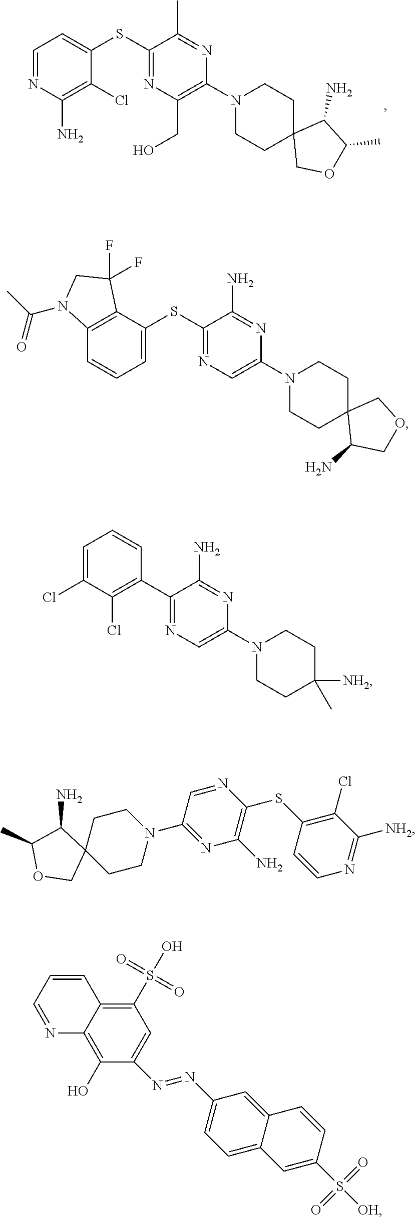
or a pharmaceutically acceptable salt thereof.

14. The method of claim 13, wherein the demyelation is caused by an injury, a hypoxic-ischaemic event, a metabolic disruption, an inherited condition, or an exposure to a toxic substance.

15. The method of claim 14, wherein the injury is a spinal cord injury, traumatic brain injury, cerebral palsy, or neuropathy.

16. The method of claim 15, wherein the hypoxic-ischaemic event is stroke, acute ischemic optic neuropathy, or other ischemia, or carbon monoxide exposure.

17. The method of claim 16, wherein the metabolic disruption is entral pontine myelolysis (CPM), or extrapontine myelinolysis (EPM).

18. The method of claim 17, wherein the inherited condition is Charcot-Marie-Tooth disease (CMT), Sjogren-Larsson syndrome, Refsum disease, Krabbe disease, Canavan disease, Alexander disease, Friedreich's ataxia, Pelizaeus-Merzbacher disease, Bassen-Kornzweig syndrome, metachromatic leukodystrophy (MLD), adrenoleukodystrophy, Leber's optic neuropathy, or nerve damage due to pernicious anemia.

19. The method of claim 18, wherein the exposure to a toxic substance is chronic alcoholism.

20. A method of treating or preventing Noonan syndrome, Leopard syndrome, Legius syndrome, Alzheimer's disease, Parkinson's disease, dementia with Lewy bodies, multiple system atrophy, Huntington's disease, spinocerebellar ataxias (e.g., SCA1, SCA2, SCA3, SCA6, SCAT, and SCA17), spinobulbar muscular atrophy (SBMA), Kennedy disease, dentatorubropallidoluysian atrophy (DRPLA), ALS, AIDS dementia, frontotemporal dementia, corticobasal ganglionic degeneration, progressive supranuclear palsy, Creutzfeldt-Jakob disease, Gerstmann-Straussler-Scheinker syndrome, fatal familial insomnia, corticobasal ganglionic degeneration, hereditary spastic paraplegia, multiple sclerosis, neuromyelitis optica (Devic's disease), concentric sclerosis (Baló's disease), acute disseminated encephalomyelitis (ADEM), acute haemorrhagic leucoencephalitis (AHL), Guillain-Barre Syndrome, chronic inflammatory demyelinating polyneuropathy (CIDP), transverse myelitis, Schilder's disease, fibromyalgia, optic neuritis, spinal cord injury, traumatic brain injury, cerebral palsy, neuropathy; stroke, acute ischemic optic neuropathy, or other ischemia, and carbon monoxide exposure; central pontine myelolysis (CPM), extrapontine myelinolysis (EPM), Charcot-Marie-Tooth disease (CMT), Sjogren-Larsson syndrome, Refsum disease, Krabbe disease, Canavan disease, Alexander disease, Friedreich's ataxia, Pelizaeus-Merzbacher disease, Bassen-Kornzweig syndrome, metachromatic leukodystrophy (MLD), adrenoleukodystrophy, Leber's optic neuropathy, nerve damage due to pernicious anemia, progressive multifocal leukoencephalopathy (PML), Lyme disease, tabes dorsalis due to untreated syphilis, HIV, subacute sclerosing panencephalitis due to measles virus, Marchiafava-Bignami disease, chemotherapy, a disorder resulting from exposure to mitochondrial toxins, or exposure to chemicals, vitamin B12 deficiency, vitamin E deficiency, copper deficiency, trigeminal neuralgia, Marchiafava-Bignami disease, or Bell's palsy in a subject in need thereof, comprising administering to the subject an effective amount of a SHP2 inhibitor having the structure: 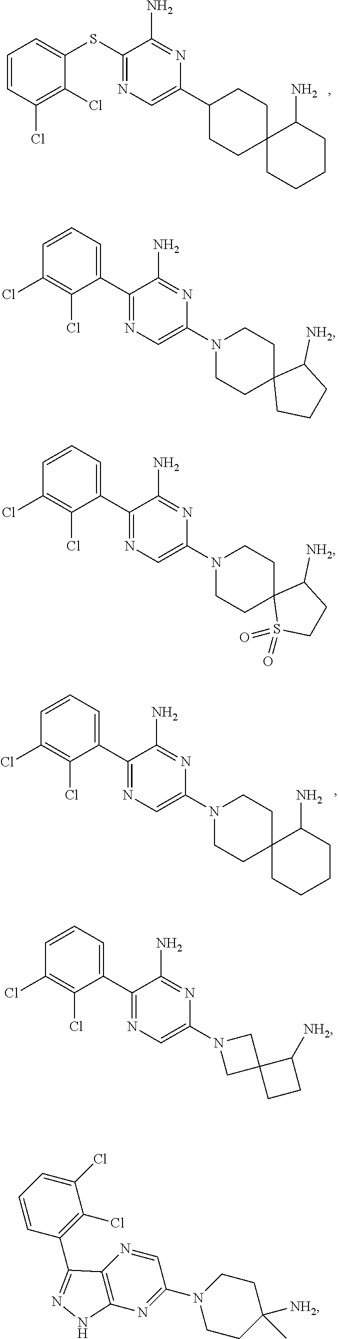 [SMILES: Nc1nc(C2CCC3(CCCCC3N)CC2)cnc1Sc1cccc(Cl)c1Cl ; Nc1nc(N2CCC3(CCCC3N)CC2)cnc1-c1cccc(Cl)c1Cl ; Nc1nc(N2CCC3(CC2)C(N)CCS3(=O)=O)cnc1-c1cccc(Cl)c1Cl ; Nc1nc(N2CCC3(CCCCC3N)CC2)cnc1-c1cccc(Cl)c1Cl ; Nc1nc(N2CC3(CCC3N)C2)cnc1-c1cccc(Cl)c1Cl ; CC1(N)CCN(c2cnc3c(-c4cccc(Cl)c4Cl)n[nH]c3n2)CC1] 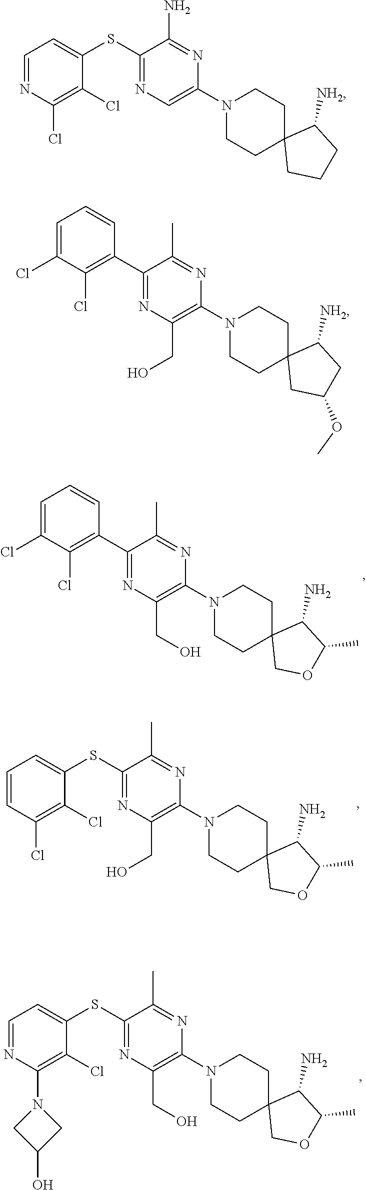 [SMILES: Nc1nc(N2CCC3(CCC[C@H]3N)CC2)cnc1Sc1ccnc(Cl)c1Cl ; CO[C@H]1C[C@@H](N)C2(CCN(c3nc(C)c(-c4cccc(Cl)c4Cl)nc3CO)CC2)C1 ; Cc1nc(N2CCC3(CC2)CO[C@@H](C)[C@H]3N)c(CO)nc1-c1cccc(Cl)c1Cl ; Cc1nc(N2CCC3(CC2)CO[C@@H](C)[C@H]3N)c(CO)nc1Sc1cccc(Cl)c1Cl ; Cc1nc(N2CCC3(CC2)CO[C@@H](C)[C@H]3N)c(CO)nc1Sc1ccnc(N2CC(O)C2)c1Cl] 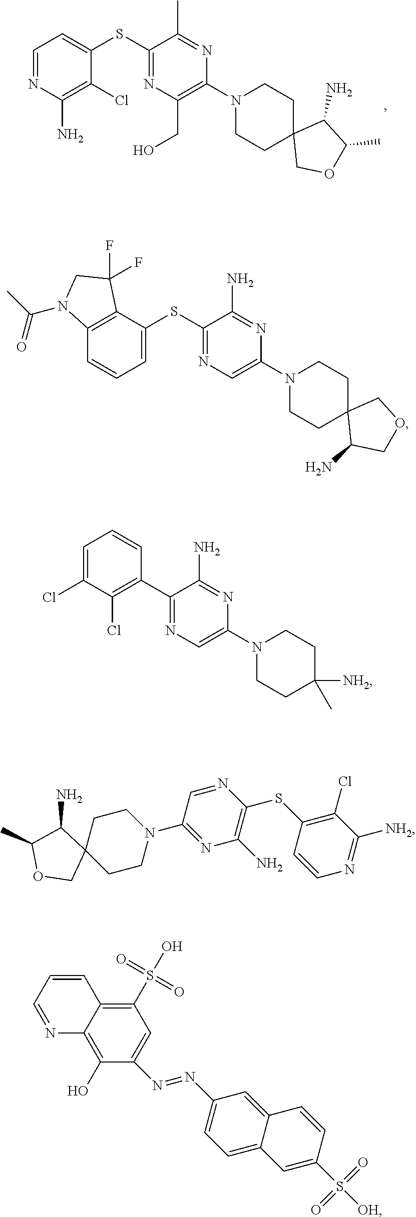
or a pharmaceutically acceptable salt thereof.

21. The method of claim 20, wherein the neuropathy is neuropathy due to diabetes, chronic renal failure, hypothyroidism, liver failure, or compression of the nerve (e.g. in Bell's palsy), or post radiation injury.

22. The method of any one of claims 1-21, wherein the SHP2 inhibitor is: 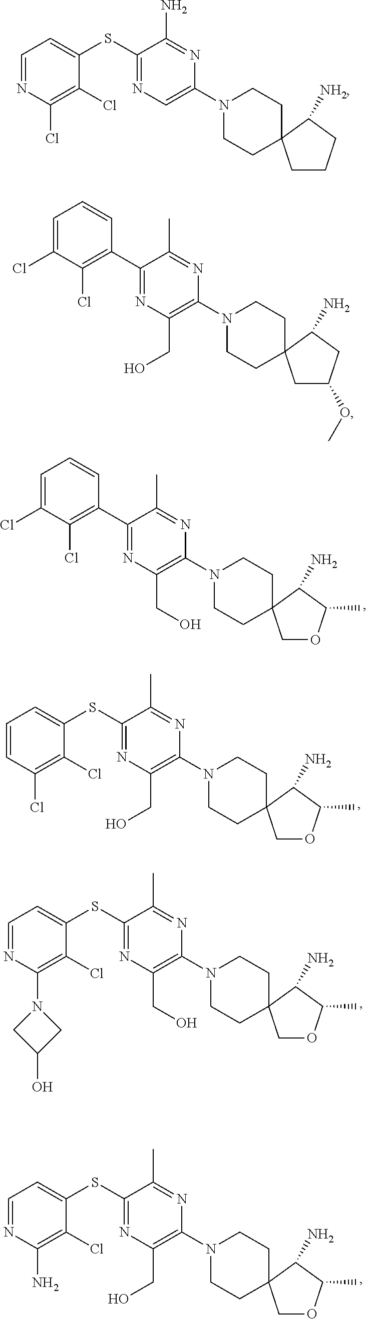 [SMILES: Nc1nc(N2CCC3(CCC[C@H]3N)CC2)cnc1Sc1ccnc(Cl)c1Cl ; CO[C@H]1C[C@@H](N)C2(CCN(c3nc(C)c(-c4cccc(Cl)c4Cl)nc3CO)CC2)C1 ; Cc1nc(N2CCC3(CC2)CO[C@@H](C)[C@H]3N)c(CO)nc1-c1cccc(Cl)c1Cl ; Cc1nc(N2CCC3(CC2)CO[C@@H](C)[C@H]3N)c(CO)nc1Sc1cccc(Cl)c1Cl ; Cc1nc(N2CCC3(CC2)CO[C@@H](C)[C@H]3N)c(CO)nc1Sc1ccnc(N2CC(O)C2)c1Cl ; Cc1nc(N2CCC3(CC2)CO[C@@H](C)[C@H]3N)c(CO)nc1Sc1ccnc(N)c1Cl]
or a pharmaceutically acceptable salt thereof.

23. The method of any one of claims 1-21, wherein the SHP2 inhibitor is: 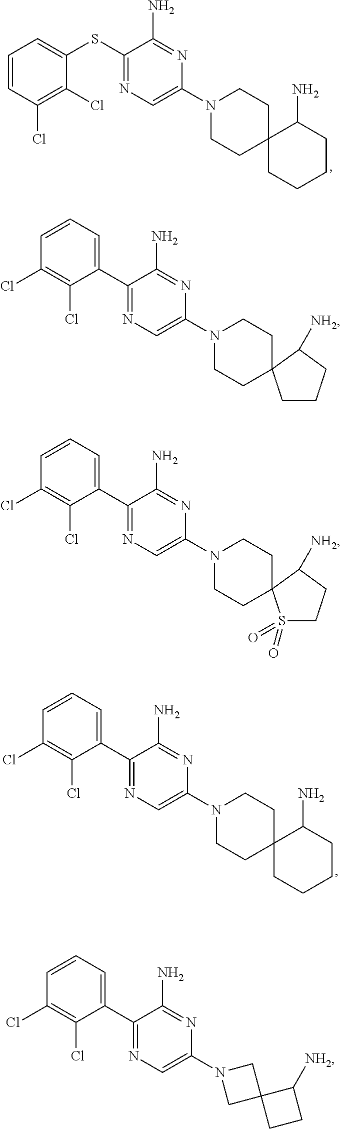 [SMILES: Nc1nc(N2CCC3(CCCCC3N)CC2)cnc1Sc1cccc(Cl)c1Cl ; Nc1nc(N2CCC3(CCCC3N)CC2)cnc1-c1cccc(Cl)c1Cl ; Nc1nc(N2CCC3(CC2)C(N)CCS3(=O)=O)cnc1-c1cccc(Cl)c1Cl ; Nc1nc(N2CCC3(CCCCC3N)CC2)cnc1-c1cccc(Cl)c1Cl ; Nc1nc(N2CC3(CCC3N)C2)cnc1-c1cccc(Cl)c1Cl]
or a pharmaceutically acceptable salt thereof.

24. The method of any one of claims 1-21, wherein the SHP2 inhibitor is: 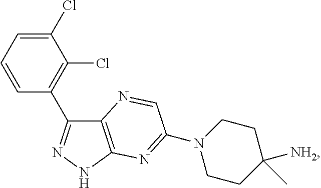 [SMILES: CC1(N)CCN(c2cnc3c(-c4cccc(Cl)c4Cl)n[nH]c3n2)CC1]
or a pharmaceutically acceptable salt thereof.

25. The method of any one of claims 1-21, wherein the SHP2 inhibitor is 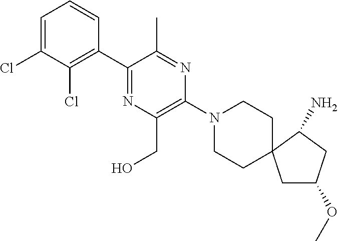 [SMILES: CO[C@H]1C[C@@H](N)C2(CCN(c3nc(C)c(-c4cccc(Cl)c4Cl)nc3CO)CC2)C1]
or a pharmaceutically acceptable salt thereof.

26. The method of any one of claims 1-21, wherein the SHP2 inhibitor is 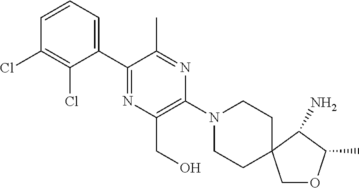 [SMILES: Cc1nc(N2CCC3(CC2)CO[C@@H](C)[C@H]3N)c(CO)nc1-c1cccc(Cl)c1Cl]
or a pharmaceutically acceptable salt thereof.

27. The method of any one of claims 1-21, wherein the SHP2 inhibitor is 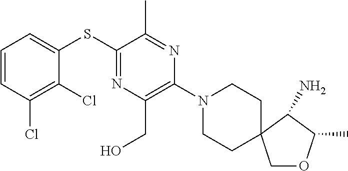 [SMILES: Cc1nc(N2CCC3(CC2)CO[C@@H](C)[C@H]3N)c(CO)nc1Sc1cccc(Cl)c1Cl]
or a pharmaceutically acceptable salt thereof.

28. The method of any one of claims 1-21, wherein the SHP2 inhibitor is 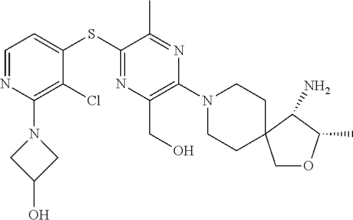 [SMILES: Cc1nc(N2CCC3(CC2)CO[C@@H](C)[C@H]3N)c(CO)nc1Sc1ccnc(N2CC(O)C2)c1Cl]
or a pharmaceutically acceptable salt thereof.

29. The method of any one of claims 1-21, wherein the SHP2 inhibitor is 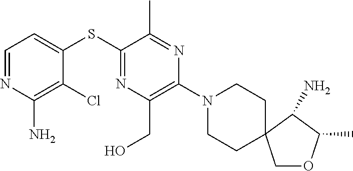 [SMILES: Cc1nc(N2CCC3(CC2)CO[C@@H](C)[C@H]3N)c(CO)nc1Sc1ccnc(N)c1Cl]
or a pharmaceutically acceptable salt thereof.

30. The method of any one of claims 1-21, wherein the SHP2 inhibitor is 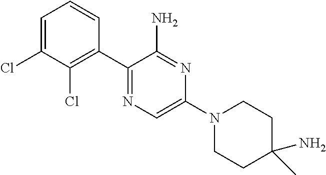 [SMILES: CC1(N)CCN(c2cnc(-c3cccc(Cl)c3Cl)c(N)n2)CC1]
or a pharmaceutically acceptable salt thereof.

31. The method of any one of claims 1-21, wherein the SHP2 inhibitor is 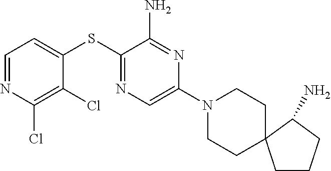 [SMILES: Nc1nc(N2CCC3(CCC[C@H]3N)CC2)cnc1Sc1ccnc(Cl)c1Cl]
or a pharmaceutically acceptable salt thereof.

32. The method of any one of claims 1-21, wherein the SHP2 inhibitor is 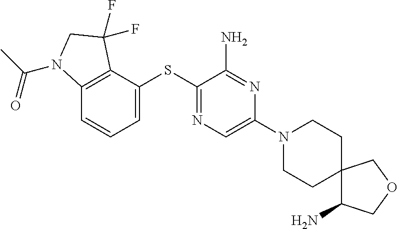 [SMILES: CC(=O)N1CC(F)(F)c2c(Sc3ncc(N4CCC5(CC4)COC[C@H]5N)nc3N)cccc21]
or a pharmaceutically acceptable salt thereof.

33. The method of any one of claims 1-21, wherein the SHP2 inhibitor is 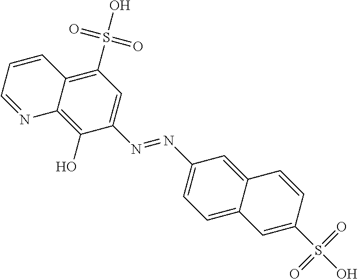 [SMILES: O=S(=O)(O)c1ccc2cc(/N=N/c3cc(S(=O)(=O)O)c4cccnc4c3O)ccc2c1]
or a pharmaceutically acceptable salt thereof.

34. The method of any one of claims 1-33, wherein the SHP2 inhibitor is administered orally, parenterally, rectally, transdermally, intradermally, intrathecally, subcutaneously, intravenously, intramuscularly, or intranasally.

35. The method of any one of claims 1-34, wherein the SHP2 inhibitor is orally administered.

36. A method of treating or preventing neuroinflammation in a subject in need thereof, the method comprising administering to the subject an effective amount of a SHP1 inhibitor.

37. A method of treating or preventing a neuroinflammatory disorder in a subject in need thereof, the method comprising administering to the subject an effective amount of a SHP1 inhibitor.

38. A method of treating or preventing a disorder associated with neuroinflammation in a subject in need thereof, the method comprising administering to the subject an effective amount of a SHP1 inhibitor.

39. The method of claim 36, wherein the neuroinflammation is associated with a separate disorder in the subject.

40. A method of treating or preventing a neurodegenerative disorder in a subject in need thereof, comprising administering to the subject an effective amount of a SHP1 inhibitor.

41. The method of claim 40, wherein the neurodegenerative disorder is Alzheimer's disease, Parkinson's disease, dementia with Lewy bodies, multiple system atrophy, Huntington's disease, spinocerebellar ataxias, spinobulbar muscular atrophy (SBMA) or Kennedy disease, dentatorubropallidoluysian atrophy (DRPLA), ALS, AIDS dementia, frontotemporal dementia, corticobasal ganglionic degeneration, progressive supranuclear palsy, Creutzfeldt-Jakob disease, Gerstmann-Straussler-Scheinker syndrome, fatal familial insomnia, corticobasal ganglionic degeneration, hereditary spastic paraplegia, multiple sclerosis, neuromyelitis optica (Devic's disease), concentric sclerosis (Baló's disease), encephalomyelitis including acute disseminated encephalomyelitis (ADEM), acute haemorrhagic leucoencephalitis (AHL), Guillain-Barre Syndrome, chronic inflammatory demyelinating polyneuropathy (CIDP), transverse myelitis, Schilder's disease, fibromyalgia, or optic neuritis.

42. A method of treating or preventing Multiple Sclerosis, Parkinson's disease, Multiple System Atrophy, Corticobasal Degeneration, Progressive Supranuclear Paresis, Guillain-Barre Syndrome (GBS), chronic inflammatory demyelinating polyneuropathy (CIDP), viral encephalitis, cerebrovascular accidents, or cranial trauma in a subject in need thereof, comprising administering to the subject an effective amount of a SHP1 inhibitor.

43. The method of claim 42, wherein the Multiple Sclerosis is Relapse Remitting Multiple Sclerosis, Secondary Progressive Multiple Sclerosis or Primary Progressive Multiple Sclerosis.

44. A method of treating or preventing a genetic disorder resulting in loss-of-function in the ras signaling pathway in a subject in need thereof, comprising administering to the subject an effective amount of a SHP1 inhibitor.

45. The method of claim 44, wherein the genetic disorder resulting in loss-of-function in the ras signaling pathway is Legius syndrome.

46. A method of treating or preventing demyelination in a subject in need thereof, comprising administering to the subject an effective amount of a SHP1 inhibitor.

47. The method of claim 46, wherein the demyelation is caused by an injury, a hypoxic-ischaemic event, a metabolic disruption, an inherited condition, or an exposure to a toxic substance.

48. The method of claim 47, wherein the injury is a spinal cord injury, traumatic brain injury, cerebral palsy, or neuropathy.

49. The method of claim 47, wherein the hypoxic-ischaemic event is stroke, acute ischemic optic neuropathy, or other ischemia, or carbon monoxide exposure.

50. The method of claim 47, wherein the metabolic disruption is entral pontine myelolysis (CPM), or extrapontine myelinolysis (EPM).

51. The method of claim 47, wherein the inherited condition is Charcot-Marie-Tooth disease (CMT), Sjogren-Larsson syndrome, Refsum disease, Krabbe disease, Canavan disease, Alexander disease, Friedreich's ataxia, Pelizaeus-Merzbacher disease, Bassen-Kornzweig syndrome, metachromatic leukodystrophy (MLD), adrenoleukodystrophy, Leber's optic neuropathy, or nerve damage due to pernicious anemia.

52. The method of claim 47, wherein the exposure to a toxic substance is chronic alcoholism.

53. A method of treating or preventing Noonan syndrome, Leopard syndrome, Legius syndrome, Alzheimer's disease, Parkinson's disease, dementia with Lewy bodies, multiple system atrophy, Huntington's disease, spinocerebellar ataxias (e.g., SCA1, SCA2, SCA3, SCA6, SCAT, and SCA17), spinobulbar muscular atrophy (SBMA), Kennedy disease, dentatorubropallidoluysian atrophy (DRPLA), ALS, AIDS dementia, frontotemporal dementia, corticobasal ganglionic degeneration, progressive supranuclear palsy, Creutzfeldt-Jakob disease, Gerstmann-Straussler-Scheinker syndrome, fatal familial insomnia, corticobasal ganglionic degeneration, hereditary spastic paraplegia, multiple sclerosis, neuromyelitis optica (Devic's disease), concentric sclerosis (Baló's disease), acute disseminated encephalomyelitis (ADEM), acute haemorrhagic leucoencephalitis (AHL), Guillain-Barre Syndrome, chronic inflammatory demyelinating polyneuropathy (CIDP), transverse myelitis, Schilder's disease, fibromyalgia, optic neuritis, spinal cord injury, traumatic brain injury, cerebral palsy, neuropathy; stroke, acute ischemic optic neuropathy, or other ischemia, and carbon monoxide exposure; central pontine myelolysis (CPM), extrapontine myelinolysis (EPM), Charcot-Marie-Tooth disease (CMT), Sjogren-Larsson syndrome, Refsum disease, Krabbe disease, Canavan disease, Alexander disease, Friedreich's ataxia, Pelizaeus-Merzbacher disease, Bassen-Kornzweig syndrome, metachromatic leukodystrophy (MLD), adrenoleukodystrophy, Leber's optic neuropathy, nerve damage due to pernicious anemia, progressive multifocal leukoencephalopathy (PML), Lyme disease, tabes dorsalis due to untreated syphilis, HIV, subacute sclerosing panencephalitis due to measles virus, Marchiafava-Bignami disease, chemotherapy, a disorder resulting from exposure to mitochondrial toxins, or exposure to chemicals, vitamin B12 deficiency, vitamin E deficiency, copper deficiency, trigeminal neuralgia, Marchiafava-Bignami disease, or Bell's palsy in a subject in need thereof, comprising administering to the subject an effective amount of a SHP1 inhibitor.

54. The method of any one of claims 36-53, wherein the SHP1 inhibitor is administered orally, parenterally, rectally, transdermally, intradermally, intrathecally, subcutaneously, intravenously, intramuscularly, or intranasally.

55. The method of any one of claims 36-54, wherein the SHP1 inhibitor is orally administered.
